SEQUENCE LISTING (75 sequences)

SEQ ID NO: 1 - protein, 139 aa, synthetic construct
QVQLQESGGGLVQAGGSLRLSCAASGRTFSEYAMGWFRQAPGREREFVATIGPSGGVTGYTDSVRGRFTISRDNARNTVYLQMNSLRPDDTAVYYCAAAGLGTXVSEWDYDYDYWGQGTQVTVSSGSGGGSGGSGGGSG

SEQ ID NO: 2 - protein, 139 aa, synthetic construct
QVQLQESGGGLVQAGGSLRLSCAASGRTFSEYAMGWFRQAPGREREFVATIGPSGGITGYTDSVRGRFTISRDNARNTVYLQMNSLRPDDTAVYYCAAAGLGTXVSEWDYDYDYWGQGTQVTVSSGSGGGSGGSGGGSG

SEQ ID NO: 3 - protein, 139 aa, synthetic construct
QVQLQESGGGLVQAGGSLRLSCAASGRTFSEYAMGWFRQAPGREREFVATILRSGGSTFYTDSVRGRFTISRDNARNTVYLQMNSLRPDDTAVYYCAAAGLGTXVSEWDYDYDYWGRGTQVTVSSGSGGGSGGSGGGSG

SEQ ID NO: 4 - protein, 141 aa, synthetic construct
QVQLVESGGGLMQAGGSLRLSCAVSGXTFSTAAMGWFRQAPGREREFVAAIRWSGGSAYYADSVRGRFTISRDRARNTVYLQMNSLRYEDTAVYYCAQTHYVSYLLSDYATWPYDYWGQGTQVTVSSGSGGSGGGSGGGSG

SEQ ID NO: 5 - protein, 144 aa, synthetic construct
QVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSSGSGGSGGGSGGGSG

SEQ ID NO: 6 - protein, 144 aa, synthetic construct
QVQLQESGGGLVQPGGSLRLSCAASGRTFSXYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSSGSGGSGGGSGGGSG

SEQ ID NO: 7 - protein, 140 aa, synthetic construct
MQVQLQESGGGLVQAGGSLRLSCAASGRTFSEXAMGWFRQAPGREREFVATISWSGGSTYYTDSVRGRFTISRDNARNTVYLQMNSLRPDDTAVYYCAAAGLGTVVSEWDYDYDYWGQGTQVTVSSGSGGGSGGSGGGSG

SEQ ID NO: 8 - protein, 140 aa, synthetic construct
MQVQLQESGGGLVQAGGSLRLSCAASGRTFSEYAMGWFRQAPGREREFVATISWSGGXTYYTDSVRGRFTISRDNARNTVYLQMNSLRPDDTAVYYCAAAGLGTVVSEWDYDYDYWGQGTQVTVSSGSGGGSGGSGGGSG

SEQ ID NO: 9 - protein, 144 aa, synthetic construct
MKIEEQVQLQESGGGLVQAGGSLRLSCAASGRTFSEYAMGWFRQAPGKEREFVATIGPSGGCTGYTDSVKGRFTISRDNAKNTVYLQMNSLKPDDTAVYYCAAAGLGTXVSEWDYDYDYWGQGTQVTVSSGSGGGSGGSGGGSG

SEQ ID NO: 10 - protein, 144 aa, synthetic construct
MKIEEQVQLQESGGGLVQAGGSLRLSCAASGRTFSEYAMGWFRQAPGKEREFVATIGPSGGITYYTDSVKGRFTISRDNAKNTVYLQMNSLKPDDTAVYYCAAAGLGVXISEWDYDYDYWGQGTQVTVSSGSGGGSGGSGGGSG

SEQ ID NO: 11 - protein, 141 aa, synthetic construct
QVQLQESGGGSVQAGGSLRLSCVVSGNTGSTGYWAWFRQGPGTEREGVAAXYTAGSGTSMTYYADSVKGRFTISQDNAKKTLYLQMNSLKPEDTGMYRCASTRFAGRWYRDSEYRAWGQGTQVTVSSGSGGSGGGSGGGSG

SEQ ID NO: 12 - protein, 140 aa, synthetic construct
QSSGQVQLQESGGGLVQPGGSLRLSCTASGVTISALNAMAMGWYRQAPGERRVMVAAVSERGNAXYRESVQGRFTVTRDFTNRMVSLQMDNLRPEDTAVYYCHVLEDRVDSFHDYWGQGTQVTVSSGSGGSGGGSGGGSG

SEQ ID NO: 13 - DNA, 5331 bp, synthetic construct
  source 1..5331: mol_type = other DNA
aaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccattaacgaattcgagctccgtcgacaagcttgcggccgcactcgagcaccaccaccaccaccactgagatccggctgctaac

SEQ ID NO: 14 - DNA, 5337 bp, synthetic construct
  source 1..5337: mol_type = other DNA
aaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgcatcaccatcatcatcatcatcactcgtcaggcgagaatctttattttcagagcagtggttaataacgaattcgagctccgtcgacaagcttgcggccgcactcgagcaccaccaccaccaccactgagatccggctgctaac

SEQ ID NO: 15 - DNA, 2251 bp, synthetic construct
  source 1..2251: mol_type = other DNA
gctagtggtgctagccccgcgaaattaatacgactcactatagggtctagaaataattttgtttaactttaagaaggagatatacatataatgaggatcccgggaattctcgagtaaggttaacctgcaggaggcctttaattaaggtggtgcggccgcgctagcggtcccgggggatcgatccggctgctaacaaagcccgaaaggaagctgagttggctgctgccaccgctgagcaataactagcataaccccttggggcctctaaacgggtcttgaggggttttttgctgaaaggaggaactatatccggaagcttggcactggccgaccggggtcgagcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccgctaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacagatccgggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 16 - DNA, 2670 bp, synthetic construct
  source 1..2670: mol_type = other DNA
gctagtggtgctagccccgcgaaattaatacgactcactatagggtctagaaataattttgtttaactttaagaaggagatatacatatgcaagtgcagttacaagagtcgggtggtggactggtccaggccggtggctctttgcgcttaagttgtgccgcttctggccgtactttttcagaatacgcaatgggatggttccgtcaagctccggggaaagaacgtgagtttgtggcgactatttcctggtctggggggtctacctactacacagacagcgtaaaaggccgtttcacaatctcgcgcgataacgcgaagaataccgtgtatctgcaaatgaattcattgaaacccgacgacaccgcagtatattattgtgcagcggcggggttgggtacgtaggtttctgagtgggattacgattacgattactggggccagggtactcaagtcaccgtaagctcaggatctggtggtagtggaggaggttctggtggtggttctggttaatgaggatcccgggaattctcgagtaaggttaacctgcaggaggcctttaattaaggtggtgcggccgcgctagcggtcccgggggatcgatccggctgctaacaaagcccgaaaggaagctgagttggctgctgccaccgctgagcaataactagcataaccccttggggcctctaaacgggtcttgaggggttttttgctgaaaggaggaactatatccggaagcttggcactggccgaccggggtcgagcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccgctaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacagatccgggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 17 - DNA, 456 bp, synthetic construct
  source 1..456: mol_type = other DNA
atgcaagtgcagttacaagagtcgggtggtggactggtccaggccggtggctctttgcgcttaagttgtgccgcttctggccgtactttttcagaatacgcaatgggatggttccgtcaagctccggggcgtgaacgtgagtttgtggcgactattndtnsgtctggcggcndtaccndttacacagacagcgtacgcggccgtttcacaatctcgcgcgataacgcgcgtaataccgtgtatctgcaaatgaattcattgcgtcccgacgacaccgcagtatattattgtgcagcggcggggttgggtnnctagnnctctgagnncgatnncgattacgattactggggccagggtactcaagtcaccgtaagctcaggatctggtggtcaccatcaccaccatcatggaagcggccatcaccatcatcatcatggttctggtggtggttctggt

SEQ ID NO: 18 - DNA, 4194 bp, synthetic construct
  source 1..4194: mol_type = other DNA
cgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcactgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgtataatatcatacgctgttatacgttgtttacgctttgaggaatcccatatggacgaattcgaaatgatcaaacgtaacacctctgaaatcatctctgaagaagaactgcgtgaagttctgaaaaaagacgaaaaatctgcggaaatcggtttcgaaccgtctggtaaaatccacctgggtcactacctgcagatcaaaaaaatgatcgacctgcagaacgcgggtttcgacatcatcatccatctgggtgacctgggagcgtacctgaaccagaaaggtgaactggacgaaatccgtaaaatcggtgactacaacaaaaaagttttcgaagcgatgggtctgaaagcgaaatacgtttacggttctgaatatcatctggacaaagactacaccctgaacgtttaccgtctggcgctgaaaaccaccctgaaacgtgcgcgtcgttctatggaactgatcgcgcgtgaagacgaaaacccgaaagttgcggaagttatctacccgatcatgcaggttaacggtatccactacggtggtgttgacgttgcggttggtggtatggaacagcgtaaaatccacatgctggcgcgtgaactgctgccgaaaaaagttgtttgcatccacaacccggttctgaccggtctggacggtgaaggtaaaatgtcttcttctaaaggtaacttcatcgcggttgacgactctccggaagaaatccgtgcgaaaatcaaaaaagcgtactgcccggcaggtgttgttgaaggtaacccgatcatggaaatcgcgaaatacttcctggaatacccgctgaccatcaaacgtccggaaaaattcggtggtgacctgaccgttaactcttacgaagaactggaatctctgttcaaaaacaaagaactgcacccgatggacctgaaaaacgcggttgcggaagaactgatcaaaatcctggaaccgatccgtaaacgtctgtaactgcagtttcaaacgctaaattgcctgatgcgctacgcttatcaggcctacatgatctctgcaatatattgagtttgcgtgcttttgtaggccggataaggcgttcacgccgcatccggcaagaaacagcaaacaatccaaaacgccgcgttcagcggcgttttttctgcttttcttcgcgaattaattccgcttcgcaacatgtgagcaccggtttattgactaccggaagcagtgtgaccgtgtgcttctcaaatgcctgaggccagtttgctcaggctctccccgtggaggtaataattgacgatatgatcagtgcacggctaactaagcggcctgctgactttctcgccgatcaaaaggcattttgctattaagggattgacgagggcgtatctgcgcagtaagatgcgccccgcattccggcggtagttcagcagggcagaacggcggactctaaatccgcatggcaggggttcaaatcccctccgccggaccaaattcgaaaagcctgctcaacgagcaggcttttttgcatgctcgagcagctcagggtcgtttcaaacgctaaattgcctgatgcgctacgcttatcaggcctacatgatctctgcaatatattgagtttgcgtgcttttgtaggccggataaggcgttcacgccgcatccggcaagaaacagcaaacaatccaaaacgccgcgttcagcggcgttttttctgcttttcttcgcgaattaattccgcttcgcacatgtgagcaaaaggccagcaaaaggccaggaaccgctcgagcgttttatctgttgtttgtcggtgaacgctctcctgagtaggacaaatccgccgggagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcat

SEQ ID NO: 19 - DNA, 141 bp, synthetic construct
  source 1..141: mol_type = other DNA
ctagccccgcgaaattaatacgactcactatagggtctagaaataattttgtttaactttaagaaggagatatacatatgttccccgtcttcgtctaatgaggatcccgggaattctcgagtaaggttaacctgcaggagg

SEQ ID NO: 20 - DNA, 141 bp, synthetic construct
  source 1..141: mol_type = other DNA
ctagccccgcgaaattaatacgactcactatagggtctagaaataattttgtttaactttaagaaggagatatacatatgtagcccgtcttcgtctaatgaggatcccgggaattctcgagtaaggttaacctgcaggagg

SEQ ID NO: 21 - DNA, 141 bp, synthetic construct
  source 1..141: mol_type = other DNA
ctagccccgcgaaattaatacgactcactatagggtctagaaataattttgtttaactttaagaaggagatatacatatgttccccgtctaggtctaatgaggatcccgggaattctcgagtaaggttaacctgcaggagg

SEQ ID NO: 22 - DNA, 381 bp, synthetic construct
  source 1..381: mol_type = other DNA
caagtgcagttacaagagtcgggtggtggactggtccaggccggtggctctttgcgcttaagttgtgccgcttctggccgtactttttcagaatacgcaatgggatggttccgtcaagctccggggaaagaacgtgagtttgtggcgactatttcctggtctggggggtctacctactacacagacagcgtaaaaggccgtttcacaatctcgcgcgataacgcgaagaataccgtgtatctgcaaatgaattcattgaaacccgacgacaccgcagtatattattgtgcagcggcggggttgggtacggtcgtttctgagtgggattacgattacgattactggggccagggtactcaagtcaccgtaagctcaggatca

SEQ ID NO: 23 - DNA, 381 bp, synthetic construct
  source 1..381: mol_type = other DNA
caagtgcagttacaagagtcgggtggtggactggtccaggccggtggctctttgcgcttaagttgtgccgcttctggccgtactttttcagaatacgcaatgggatggttccgtcaagctccggggaaagaacgtgagtttgtggcgactatttcctggtctggggggtctacctactacacagacagcgtaaaaggccgtttcacaatctcgcgcgataacgcgaagaataccgtgtatctgcaaatgaattcattgaaacccgacgacaccgcagtatattattgtgcagcggcggggttgggtacggtcgtttctgagtaggattacgattacgattactggggccagggtactcaagtcaccgtaagctcaggatca

SEQ ID NO: 24 - DNA, 417 bp, synthetic construct
  source 1..417: mol_type = other DNA
caagtgcagttacaagagtcgggtggtggactggtccaggccggtggctctttgcgcttaagttgtgccgcttctggccgtactttttcagaatacgcaatgggatggttccgtcaagctccggggcgtgaacgtgagtttgtggcgactatttcctggtctggggggtctacctactacacagacagcgtacgcggccgtttcacaatctcgcgcgataacgcgcgtaataccgtgtatctgcaaatgaattcattgcgtcccgacgacaccgcagtatattattgtgcagcggcggggttgggtacgaaagtttctgagtgggattacgattacgattactggggccagggtactcaagtcaccgtaagctcaggatctggtggtagtggaggaggttctggtggtggttctggt

SEQ ID NO: 25 - DNA, 141 bp, synthetic construct; identical to SEQ ID NO: 19
  source 1..141: mol_type = other DNA

SEQ ID NO: 26 - DNA, 141 bp, synthetic construct; identical to SEQ ID NO: 20
  source 1..141: mol_type = other DNA

SEQ ID NO: 27 - DNA, 141 bp, synthetic construct; identical to SEQ ID NO: 21
  source 1..141: mol_type = other DNA

SEQ ID NO: 28 - DNA, 417 bp, synthetic construct
  source 1..417: mol_type = other DNA
caagtgcagttacaagagtcgggtggtggactggtccaggccggtggctctttgcgcttaagttgtgccgcttctggccgtactttttcagaatacgcaatgggatggttccgtcaagctccggggaaagaacgtgagtttgtggcgactattggtccgtctggcggcgttaccggttacacagacagcgtaaaaggccgtttcacaatctcgcgcgataacgcgaagaataccgtgtatctgcaaatgaattcattgaaacccgacgacaccgcagtatattattgtgcagcggcggggttgggtacgtaggtttctgagtgggattacgattacgattactggggccagggtactcaagtcaccgtaagctcaggatctggtggtggaagcggcggttctggtggtggttctggt

SEQ ID NO: 29 - DNA, 417 bp, synthetic construct
  source 1..417: mol_type = other DNA
caagtgcagttacaagagtcgggtggtggactggtccaggccggtggctctttgcgcttaagttgtgccgcttctggccgtactttttcagaatacgcaatgggatggttccgtcaagctccggggaaagaacgtgagtttgtggcgactattggtccgtctggcggcattaccggttacacagacagcgtaaaaggccgtttcacaatctcgcgcgataacgcgaagaataccgtgtatctgcaaatgaattcattgaaacccgacgacaccgcagtatattattgtgcagcggcggggttgggtacgtaggtttctgagtgggattacgattacgattactggggccagggtactcaagtcaccgtaagctcaggatctggtggtggaagcggcggttctggtggtggttctggt

SEQ ID NO: 30 - DNA, 417 bp, synthetic construct
  source 1..417: mol_type = other DNA
caagtgcagttacaagagtcgggtggtggactggtccaggccggtggctctttgcgcttaagttgtgccgcttctggccgtactttttcagaatacgcaatgggatggttccgtcaagctccggggcgtgaacgtgagtttgtggcgactattcttcggtctggcggcagtaccttttacacagacagcgtacgcggccgtttcacaatctcgcgcgataacgcgcgtaataccgtgtatctgcaaatgaattcattgcgtcccgacgacaccgcagtatattattgtgcagcggcggggttgggtacgtaggtttctgagtgggattacgattacgattactggggccgcggtactcaagtcaccgtaagctcaggatctggtggtggaagcggcggttctggtggtggttctggt

SEQ ID NO: 31 - DNA, 417 bp, synthetic construct
  source 1..417: mol_type = other DNA
caagtgcagttacaagagtcgggtggtggactggtccaggccggtggctctttgcgcttaagttgtgccgcttctggccgtactttttcagaatacgcaatgggatggttccgtcaagctccggggcgtgaacgtgagtttgtggcgactattggtccgtctggcggcgttaccggttacacagacagcgtacgcggccgtttcacaatctcgcgcgataacgcgcgtaataccgtgtatctgcaaatgaattcattgcgtcccgacgacaccgcagtatattattgtgcagcggcggggttgggtacgaaagtttctgagtgggattacgattacgattactggggccagggtactcaagtcaccgtaagctcaggatctggtggtggaagcggcggttctggtggtggttctggt

SEQ ID NO: 32 - DNA, 417 bp, synthetic construct
  source 1..417: mol_type = other DNA
caagtgcagttacaagagtcgggtggtggactggtccaggccggtggctctttgcgcttaagttgtgccgcttctggccgtactttttcagaatacgcaatgggatggttccgtcaagctccggggcgtgaacgtgagtttgtggcgactattggtccgtctggcggcattaccggttacacagacagcgtacgcggccgtttcacaatctcgcgcgataacgcgcgtaataccgtgtatctgcaaatgaattcattgcgtcccgacgacaccgcagtatattattgtgcagcggcggggttgggtacgaaagtttctgagtgggattacgattacgattactggggccagggtactcaagtcaccgtaagctcaggatctggtggtggaagcggcggttctggtggtggttctggt

SEQ ID NO: 33 - DNA, 417 bp, synthetic construct
  source 1..417: mol_type = other DNA
caagtgcagttacaagagtcgggtggtggactggtccaggccggtggctctttgcgcttaagttgtgccgcttctggccgtactttttcagaatacgcaatgggatggttccgtcaagctccggggaaagaacgtgagtttgtggcgactattcttcggtctggcggcagtaccttttacacagacagcgtaaaaggccgtttcacaatctcgcgcgataacgcgaagaataccgtgtatctgcaaatgaattcattgaaacccgacgacaccgcagtatattattgtgcagcggcggggttgggtacgaaagtttctgagtgggattacgattacgattactggggcaagggtactcaagtcaccgtaagctcaggatctggtggtggaagcggcggttctggtggtggttctggt

SEQ ID NO: 34 - DNA, 423 bp, synthetic construct
  source 1..423: mol_type = other DNA
caagtccaattagtcgagagtggcggtggtcttatgcaagccggagggagtttacgcctttcctgcgcggtatcggggcgtactttcagtactgcggcgatgggctggttccgtcaagcgcccggaaaagagcgcgagttcgtcgctgctattcgctggtcggggggctcagcatattacgctgatagcgtaaagggacgcttcaccatttcgcgtgacaaggctaaaaatactgtatatctgcagatgaactcactgaaatacgaggacacagctgtctactattgtgcccagacacattacgtgtcgtacttgttaagtgattatgcaacctggccctacgactactggggccaaggaactcaggtaacggtttcatcgggatctggtggtagtggaggaggttctggtggtggttctggt

SEQ ID NO: 35 - DNA, 405 bp, synthetic construct
  source 1..405: mol_type = other DNA
gaggtgcaactgcaggctagcgggggtggattagtccgccctggtggctcgcttcgcttgagctgcgctgcaagcggttttaccttttcatcatacgccatgatgtgggtccgtcaggcaccgggtaaggggcttgaatgggtatctgcgatcaacggaggcggaggttcgacgagctatgcagatagtgtaaaaggacgcttcaccatttcacgtgataatgcaaagaatacattgtaccttcagatgaactccctgaaaccggaggacacagccgtctattactgcgctaagtaccaggctgcagtacaccaagagaaggaagactactggggtcaaggcacgcaggtaaccgtatcgtctggatctggtggtagtggaggaggttctggtggtggttctggt

SEQ ID NO: 36 - DNA, 171 bp, synthetic construct
  source 1..171: mol_type = other DNA
atggataaagaatggattcttcaaaaaatctacgagatcatgcgtttgttagacgagctgggccacgcggaagcgagtatgcgcgtttcagatttaatctacgagtttatgaagaaaggtgatgaacgtctgttggaggaagcggaacgtctgcttgaggaagtagagcgc

SEQ ID NO: 37 - DNA, 195 bp, synthetic construct
  source 1..195: mol_type = other DNA
atgaacgatgatgagctgcatatgttaatgacggatcttgtgtatgaggcactgcattttgctaaggatgaagagatcaagaagcgcgtattccaactttttgaattggccgacaaagcctacaaaaacaatgaccgtcaaaaacttgaaaaggtggttgaggaattgaaggagttattagaacgcttattgtca

SEQ ID NO: 38 - DNA, 402 bp, synthetic construct
  source 1..402: mol_type = other DNA
caattacaggagagcggcggaggattggtacagcccggaggatcgctgcgcttaagttgtactgcaagtggagttacgatttcggccctgaacgctatggcaatgggctggtatcgccaagcaccgggtgagcgccgtgtgatggtagctgccgtgtccgaacgtggcaatgctatgtaccgtgaatcggtccaaggtcgttttacagtgacacgcgatttcacaaataaaatggtgtcattacagatggacaatcttaaacccgaggataccgcagtctactattgccacgtccttgaagatcgcgtagactcctttcacgattattggggccagggaacccaggttacagtcagctctggatctggtggtagtggaggaggttctggtggtggttctggt

SEQ ID NO: 39 - DNA, 432 bp, synthetic construct
  source 1..432: mol_type = other DNA
caggtacagctgcaagaatctggaggaggcctggtacaacccggtgggagcctgcggttatcttgcgcagcttctggtcgtaccttttcaagttatgcgatggggtggttccgtcaggccccgggaaaacagagagaattcgtggccgccatacgttggtcgggcggctacacatactatacggattcagtgaagggccggttcaccatttcccgtgataatgcgaagaccaccgtgtacctgcagatgaattcccttaagcctgaagataccgcggtgtactactgcgctgccacatacctgagcagcgactatagtcggtacgctctcccacaacgtccactcgattacgattattggggacagggaacacaggtcacagtaagctcaggtagtggcggttcaggcgggggttctggtggaggctccggg

SEQ ID NO: 40 - DNA, 423 bp, synthetic construct
  source 1..423: mol_type = other DNA
caggtgcaattacaggagtctggagggggctcggtccaggcggggggctcgttacgccttagctgtgtagttagcggtaacacaggatctaccggctattgggcatggtttcgtcaaggcccaggaaccgaacgcgagggggtcgctgctacttatacagcagggtcaggcacgtcaatgacttactatgcggactcggtgaaaggacgcttcactattagccaggacaatgcaaaaaaaacgttgtatttgcagatgaactctctgaaacctgaggataccggaatgtaccgttgcgcttcaactcgcttcgcgggccgttggtaccgcgactctgaatatcgcgcttggggtcagggaacccaggttacggtatcgagtggatctggtggtagtggaggaggttctggtggtggttctggt

SEQ ID NO: 41 - DNA, 9053 bp, synthetic construct
  source 1..9053: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctaagctttaattactttaaccattgctaccttcgttgaaggtgcctgatctgtaattaccttttgaggtttaccaaattgtttaatgagacgtttgataaacgcatatgctgaatgattatctcgttgcttacgcaaccaaatatctaatgtatgtccctctgcatcaatggcacgatataaatagctccattttccttttattttgatgtacgtctcatcaatacgccatttgtaataagcttttttatgctttttcttccaaatttgatataaaattggggcatattcttgaacccaacggtagaccgttgaatgatgaacgtttacaccacgtccccttaatatttcagatatatcacgataactcaatgcatatcttagatagtagccaacggctacagtgataacatccttgttaaattgtttatatctgaaatagttcatacagaagactcctttttgttaaaattatactataaattcaactttgcaacagaaccgtaaagattatacacctttttaggtgtataatctcttttttttgaaaatttaacttcaaaaactaattaaatagagataatggattgttttccatattttttcttctcataatagttgagtgcatttcttcaagttcagcaatgatacttctcattaaataaccttccatattagctactttttcttgttttttaacaagataacctttaaatcgttttaaaaccaatagtaattcttcatctatatcttctaacatatagaaagtatcgtatttgttattaaatgattttttagcttttaaaataacagatttaatacttttaacttcttcatagctgaaattattaatataacttttaattaattcggggagttcttgaagttctatatatttaagagattctttatcatgaatatttgaaaagtgatttgaatgatttgaatgttgatttgtatcattcatattattcatatcattactttcagtatcaataaaatcagtatcaataaaatcagtatcatttgtgtgtccttttgacatttctagacgtgtccttttgacatttctagacgtgtccttttgacatttctggacgtgtccttttgacacttccttgtcttgtaaggcctcaacttcattttcagccttatctatttcataaatatcatttttagttatggctggttttaataaataaagtagatttggtttgtttaaaccctgccttttttggattagtaaatctacattttctaattcttttttaattttagtgatttttttgttcccacaatttaatatcacttctaaatcagcaactgtataaatgaaatatatgttaccttctgtatctatccagttatttttaatagataattgtaaacgatctctcaatattgcgtaagcaattttagcgtcattcgataaatctttataattaggattagtaaaaaatactttaggtaattggtaaaagcgttctttataattttcttctactgtaaaaaattgtttagacatgataaaaactcctttaaatgtatatttaaggagtttaagtaatatcgttatttacaagtgatgaataaggttatataatgttatccgttatatataacttaaagacttgttgcttttatcttgtaatgtatcgaggttgtatatataataaagatacaaacaaattgtaaaagtaaataacacatattatttaaactctttagacatctaacaaccgccaagcgttaggtgtctttttctatgttcaatagtaacactattttaaaaaaattaaaaattttttgttgcctcacaggtcaattatatgcttgtaatgaaatgtatttgataacaaatctctaatataattaatatatctaaaattatgaggccgataaatcatgaatgaataccgtatacaaaaactatatggatacagatcccatataatttttgaagcttgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatcaaatcgtctccctccgtttgaatatttgattgatcgtaaccagatcaagcactctttccactatccctacagtgttatggcttgaacaatcacgaaacaataattggtacgtacgatctttcagccgactcaaacatcaaatcttacaaatgtagtctttgaaagtattacatatgtaagatttaaatgcaaccgttttttcggaaggaaatgatgacctcgtttccaccgaattagcttgaaatagtacataatggatttccttacgcgaaatacgggcagacatggcctgcccggttattattatttttgacaccgcatgctgcggtaccgttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattcctaggtgggcccaataaaagcaatcaatgaaccaagacagcatcgatcctctagagtcaaatgtgagcagtaacaacctctgctaaaattcctgaaaaattttgcaaaaagttgttgactttatctacaaggtgtggcataatgtgtggaattgtgagcgctcacaattgacctgcaggcatgcctgcaggtcgacacataaggaggaggtaccttgaaaaagaaaaacatttattcaattcgtaaactaggtgtaggtattgcatctgtaactttaggtacattacttatatctggtggcgtaacacctgctgcaaatgctgcgccgagccgcttagaagaggaattaagaagaagattaacagaaccggcgcaacacgatgaagctcaacaaaatgctttttatcaagtcttaaatatgcctaacttaaatgctgatcaacgcaatggttttatccaaagccttaaagatgatccaagccaaagtgctaacgttttaggtgaagctcaaaaacttaatgactctcaagctccaaaagctgatgcgcaacaaaataacttcaacaaagatcaacaaagcgccttctatgaaatcttgaacatgcctaacttaaacgaagcgcaacgtaacggcttcattcaaagtcttaaagacgacccaagccaaagcactaacgttttaggtgaagctaaaaaattaaacgaatctcaagcaccgaaagctgataacaatttcaacaaagaacaacaaaatgctttctatgaaatcttgaatatgcctaacttaaacgaagaacaacgcaatggtttcatccaaagcttaaaagatgacccaagccaaagtgctaacctattgtcagaagctaaaaagttaaatgaatctcaagcaccgaaagcggataacaaattcaacaaagaacaacaaaatgctttctatgaaatcttacatttacctaacttaaacgaagaacaacgcaatggtttcatccaaagcctaaaagatgacccaagccaaagcgctaaccttttagcagaagctaaaaagctaaatgatgctcaagcaccaaaagctgacaacaaattcaacaaagaacaacaaaatgctttctatgaaattttacatttacctaacttaactgaagaacaacgtaacggcttcatccaaagccttaaagacgatccttcagtgagcaaagaaattttagcagaagctaaaaagctaaacgatgctcaagcaccaaaagaggaagacaataacaagcctggcaaagaagacaataacaagcctggcaaagaagacaacaacaagcctggtaaagaagacaacaacaagcctggtaaagaagacaacaacaagcctggcaaagaagacggcaacaagcctggtaaagaagacaacaaaaaacctggtaaagaagatggcaacaagcctggtaaagaagacaacaaaaaacctggtaaagaagacggcaacaagcctggcaaagaagatggcaacaaacctggtaaagaagatggtaacggagtacatgtcgttaaacctggtgatacagtaaatgacattgcaaaagcaaacggcactactgctgacaaaattgctgcagataacaaattagctgataaaaacatgatcaaacctggtcaagaacttgttgttgataagaagcaaccagcaaaccatgcagatgctaacaaagctcaagcattaccagaaactggtgaagaaaatccattcatcggtacaactgtatttggtggattatcattagccttaggtgcagcgttattagctggacgtcgtcgcgaactataaggatccccgggcgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 42 - DNA, 132 bp, synthetic construct
  source 1..132: mol_type = other DNA
gctccggggaaagaacgtgagtttgtggcgrscattrvcnnctctgggrscancaccwactacacagacagcgtaaaaggccgtttcacaatctcgcgcgataacgcgaagaataccgtgtatctgcaaatg

SEQ ID NO: 43 - DNA, 138 bp, synthetic construct
  source 1..138: mol_type = other DNA
ccctggccccagtaatcgtaatcgnnatcgnnctcagagnnctagnnacccaaccccgccgctgcacaataatatactgcggtgtcgtcgggtttcaatgaattcatttgcagatacacggtattcttcgcgttatcg

SEQ ID NO: 44 - DNA, 132 bp, synthetic construct
  source 1..132: mol_type = other DNA
gctccggggaaagaacgtgagtttgtggcgrscattrvctggtctgggrscancaccwactacacagacagcgtaaaaggccgtttcacaatctcgcgcgataacgcgaagaataccgtgtatctgcaaatg

SEQ ID NO: 45 - DNA, 138 bp, synthetic construct
  source 1..138: mol_type = other DNA
ccctggccccagtaatcgtaatcgnnatcccactcagagnnctagnnacccaaccccgccgctgcacaataatatactgcggtgtcgtcgggtttcaatgaattcatttgcagatacacggtattcttcgcgttatcg

SEQ ID NO: 46 - DNA, 21 bp, synthetic construct
  source 1..21: mol_type = other DNA
gctccggggaaagaacgtgag

SEQ ID NO: 47 - DNA, 24 bp, synthetic construct
  source 1..24: mol_type = other DNA
ccctggccccagtaatcgtaatcg

SEQ ID NO: 48 - DNA, 23 bp, synthetic construct
  source 1..23: mol_type = other DNA
cgattacgattactggggccagg

SEQ ID NO: 49 - DNA, 21 bp, synthetic construct
  source 1..21: mol_type = other DNA
ctcacgttctttccccggagc

SEQ ID NO: 50 - DNA, 52 bp, synthetic construct
  source 1..52: mol_type = other DNA
gtctggcggcndtaccndttacacagacagcgtaaaaggccgtttcacaatc

SEQ ID NO: 51 - DNA, 52 bp, synthetic construct
  source 1..52: mol_type = other DNA
ggtahngccgccagacsnahnaatagtcgccacaaactcacgttctttcccc

SEQ ID NO: 52 - DNA, 25 bp, synthetic construct
  source 1..25: mol_type = other DNA
ctagccccgcgaaattaatacgact

SEQ ID NO: 53 - DNA, 25 bp, synthetic construct
  source 1..25: mol_type = other DNA
cctcctgcaggttaaccttactcga

SEQ ID NO: 54 - RNA, 70 nt, synthetic construct
  source 1..70: mol_type = other RNA
ggaaacctgatcatgtagatcgaacggactctaaatccgttcagccgggttagattcccggggtttccgc

SEQ ID NO: 55 - RNA, 73 nt, synthetic construct
  source 1..73: mol_type = other RNA
gggagagtagttcaatggtagaacgtcggtctctaaaaccgagcgttgagggttcgattcctttctctcccac

SEQ ID NO: 56 - DNA, 92 bp, synthetic construct
  source 1..92: mol_type = other DNA
ttaatacgactcactatagggtctagaaataattttgtttaactttaagaaggagatatacatatgcaagtgcagttacaagagtcgggtgg

SEQ ID NO: 57 - DNA, 89 bp, synthetic construct
  source 1..89: mol_type = other DNA
accagaaccaccaccagaaccatgatgatgatggtgatggccgcttccatgatggtggtgatggtgaccaccagatcctgagcttacgg

SEQ ID NO: 58 - DNA, 30 bp, synthetic construct
  source 1..30: mol_type = other DNA
accagaaccaccaccagaaccatgatgatg

SEQ ID NO: 59 - DNA, 55 bp, synthetic construct
  source 1..55: mol_type = other DNA
gtttaactttaagaaggagatatacatatgcaagtgcagttacaagagtcgggtg

SEQ ID NO: 60 - DNA, 60 bp, synthetic construct
  source 1..60: mol_type = other DNA
cttactcgagaattcccgggatcctcattaaccagaaccaccaccagaaccatgatgatg

SEQ ID NO: 61 - DNA, 21 bp, synthetic construct
  source 1..21: mol_type = other DNA
taatgaggatcccgggaattc

SEQ ID NO: 62 - DNA, 41 bp, synthetic construct
  source 1..41: mol_type = other DNA
catatgtatatctccttcttaaagttaaacaaaattatttc

SEQ ID NO: 63 - protein, 5 aa, synthetic construct
  SITE 1..2: note = may be bound by NBD
MPVFV

SEQ ID NO: 64 - protein, 5 aa, synthetic construct
  SITE 4..5: note = may be bound by NBD
MFPVV

SEQ ID NO: 65 - protein, 6 aa, synthetic construct
MFPVFV

SEQ ID NO: 66 - protein, 4 aa, synthetic construct
PVFV

SEQ ID NO: 67 - protein, 4 aa, synthetic construct
PFPV

SEQ ID NO: 68 - protein, 104 aa, synthetic construct
  VARIANT 53: note = May be N, S, I, H, R, L, D, G, V, Y, or C
  VARIANT 54: note = May be R, T, P, G, A, W, or S
  VARIANT 58: note = May be N, S, I, H, R, L, D, G, V, Y, or C
  VARIANT 60: note = May be N, S, I, H, R, L, D, G, V, Y, or C
  VARIANT 104: note = May be N, T, S, I, H, P, R, L, D, A, G, V, Y, C, or F
MQVQLQESGGGLVQAGGSLRLSCAASGRTFSEYAMGWFRQAPGKEREFVATIXXSGGXTXYTSAVKGRFTISRDNAKNTVYLQMNSLKPDDTAVYYCAAAGLGX

SEQ ID NO: 69 - protein, 48 aa, synthetic construct
  VARIANT 1: note = May be N, T, S, I, H, P, R, L, D, A, G, V, Y, C, or F
  VARIANT 4: note = May be N, T, S, I, H, P, R, L, D, A, G, V, Y, C, or F
  VARIANT 7: note = May be N, T, S, I, H, P, R, L, D, A, G, V, Y, C, or F
XSEXKDXDYDYWGQGTGVTVSSGSGGHHHHHHGSGHHHHHHGSGGGSG

SEQ ID NO: 70 - DNA, 456 bp, synthetic construct
  source 1..456: mol_type = other DNA
atgcaagtgcagttacaagagtcgggtggtggactggtccaggccggtggctctttgcgcttaagttgtgccgcttctggccgtactttttcagaatacgcaatgggatggttccgtcaagctccggggaaagaacgtgagtttgtggcgactattndtnsgtctggcggcndtaccndttacacagacagcgtaaaaggccgtttcacaatctcgcgcgataacgcgaagaataccgtgtatctgcaaatgaattcattgaaacccgacgacaccgcagtatattattgtgcagcggcggggttgggtnnctagnnctctgagnncgatnncgattacgattactggggccagggtactcaagtcaccgtaagctcaggatctggtggtcaccatcaccaccatcatggaagcggccatcaccatcatcatcatggttctggtggtggttctggt

SEQ ID NO: 71 - protein, 10 aa, synthetic construct
TISWSGGSTY

SEQ ID NO: 72 - protein, 10 aa, synthetic construct
TIGPSGGVTG

SEQ ID NO: 73 - protein, 10 aa, synthetic construct
TIGPSGGITG

SEQ ID NO: 74 - protein, 10 aa, synthetic construct
TILRSGGSTF

SEQ ID NO: 75 - protein, 13 aa, synthetic construct
  SITE 7: note = may be modified by NBD
AAGLGTKVSEWDY

(19) United States
(12) Patent Application Publication
(10) Pub. No.: US 2026/0139001 A1
(43) Pub. Date: May 21, 2026

(54) EVOLUTION OF FLUOROGENIC SENSORS

(71) Applicants: President and Fellows of Harvard College, Cambridge, MA (US); Massachusetts Institute of Technology, Cambridge, MA (US)

(72) Inventors: Erkin Kuru, Cambridge, MA (US); George M. Church, Cambridge, MA (US); Jonathan Rittichier, Cambridge, MA (US); Helena de Puig Guixe, Cambridge, MA (US); Isaac Han, Cambridge, MA (US); Subhrajit Rout, Cambridge, MA (US); Daniel J. Wiegand, Cambridge, MA (US); James J. Collins, Newton, MA (US)

(73) Assignees: President and Fellows of Harvard College, Cambridge, MA (US); Massachusetts Institute of Technology, Cambridge, MA (US)

(21) Appl. No.: 19/116,340

(22) PCT Filed: Sep. 28, 2023
(86) PCT No.: PCT/US2023/075369

§ 371 Date: Mar. 27, 2025

Related U.S. Application Data
(60) Provisional application No. 63/410,998, filed on Sep. 28, 2022.

(51) Int. Cl. C07H 21/02; C07H 1/00; C07H 23/00; G01N 21/64; G01N 33/569; G01N 33/58; G01N 33/74

(52) U.S. Cl. CPC C07H 21/02; C07H 1/00; C07H 23/00; G01N 21/6428; G01N 33/56983; G01N 33/582; G01N 33/74; G01N 2021/6439; G01N 2333/165; G01N 2333/71

(57) ABSTRACT

Described herein is evolution strategy that leverages highly efficient tRNA charging chemistry for cell-free ribosomal translation of proteins, including fluorogenic sensors. The fluorogenic sensors provided are capable of detecting targets, including antigens such as SARS-CoV-2 variants (e.g., Omicron variants).

### RELATED APPLICATIONS

This application claims priority under 35 U.S.C. § 119(e) to U.S. Provisional Patent Application, U.S. Ser. No. 63/410,998, filed Sep. 28, 2022, the entire contents of which is incorporated herein by reference.

### GOVERNMENT SUPPORT

This invention was made with government support under DE-FG02-02ER63445 awarded by U.S. Department of Energy (DOE). The government has certain rights in this invention.

### BACKGROUND

The rapid development and refinement of fast, simple, and low-cost biosensors can enable basic research and help manage disease outbreaks.¹ Specific optical biosensors can be engineered by modification of protein binders with fluorogenic probes to sensitively detect a variety of molecular targets.²⁻⁶ Stream-lined technologies to raise protein binders against a plethora of targets have been described.^{7,8} However, their transformation into biosensors is hampered by methods that rely on low-throughput and suboptimal chemical coupling of probes, which also limits the realization of directed biosensor evolution approaches.¹

### SUMMARY OF THE INVENTION

Certain fluorescent molecules are conditionally fluorescent, i.e., “fluorogenic,” because they can selectively “turn on” (e.g., increase/decrease in fluorescence and/or change their fluorescence lifetime) upon the occurrence of a chemical or physical event. Examples of such events include changes in viscosity and local dipole environment (polarity). Both of these changes, viscosity and polarity, can occur at protein-protein binding interfaces (e.g., protein-antigen binding interfaces). In the case of protein-antigen binding, for example, selectively conjugating fluorogenic molecules at or around the binding domains of antigen-binding proteins (e.g., nanobodies) can provide fluorogenic sensors that detect protein-antigen binding, e.g., with low background fluorescence or distinct fluorescence lifetime.

A platform for preparing and employing fluorogenic sensors is described in International PCT Patent Application No. PCT/US2022/021878, published as WO 2022/204475 A1, the entire contents of which is incorporated herein by reference.

The present disclosure relates, at least in part, to new fluorogenic sensors and new methods for preparing the same. For example, described herein is an evolution strategy that leverages highly efficient tRNA charging chemistry for cell-free ribosomal translation of proteins, including fluorogenic sensors. This evolution platform allows rapid molecular design of biosensors with applications in diagnostics, bio-surveillance, and molecular imaging.

The present disclosure in one aspect provides improved methods for chemically acylating nucleotides (e.g., pdCpA) with acyl moieties such as non-standard amino acids. The acylated (i.e., “charged”) nucleotides are building blocks of charged tRNA for use in the translation of proteins, such as the fluorogenic sensors provided herein.

In one aspect, provided herein are methods of selectively acylating pdCpA at the 2′-OH and/or 3′-OH positions comprising reacting pdCpA with an acylimidazole, wherein the step of reacting is carried out in a solvent comprising water. In certain embodiments, the reaction is selective for the 2′-OH position of pdCpA. In certain embodiments, the reaction is selective for the 3′-OH position of pdCpA.

In certain embodiments, provided herein is a method of selectively acylating pdCpA at the 2′-OH and/or 3′-OH positions to form the following:

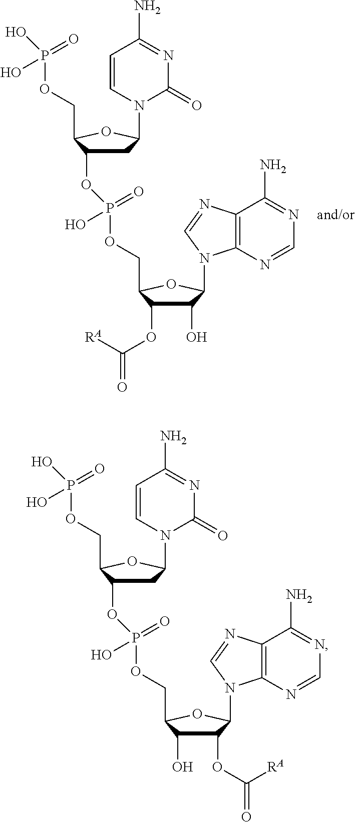

or salts thereof, the method comprising:
   (a) a step of reacting a compound of the formula: R^{A}(═O)OH, or a salt thereof, with carbonyldiimidazole (CDI) to form a compound of Formula (A):

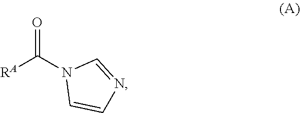

or a salt thereof; and
   (b) a step of reacting the compound of Formula (A), or a salt thereof, with pdCpA:

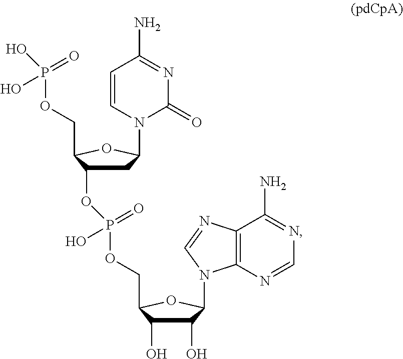

or a salt thereof,
   wherein step (b) of reacting is carried out in a solvent comprising water; and
   wherein R^{A} is an organic small molecule.

In certain embodiments, the compound R^{A}(═O)OH is a fluorogenic amino acid (FgAA). In certain embodiments, the group R^{A} is of the formula:

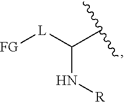

wherein:
   FG is a fluorogenic small molecule;
   L is a bond or a linker; and
   R is hydrogen or a nitrogen protecting group.

Also provided herein is a method of preparing a compound of Formula (I):

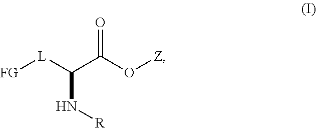

or a salt, stereoisomer, or tautomer thereof, wherein:
   FG is a fluorogenic small molecule;
   L is a bond or a linker;
   R is hydrogen or a nitrogen protecting group; and
   Z is a nucleotide;
comprising coupling a compound of Formula (II):

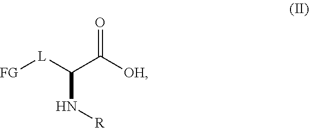

or a salt, stereoisomer, or tautomer thereof, with a nucleotide.

In certain embodiments, the compound of Formula (II) is coupled selectively at the 2′-OH and/or 3′-OH position of the nucleotide. In certain embodiments, Z is a mononucleotide, dinucleotide, or polynucleotide. In certain embodiments, Z is a dinucleotide (e.g., pdCpA). In certain embodiments, Z is pdCpA.

In certain embodiments, Z is of the formula:

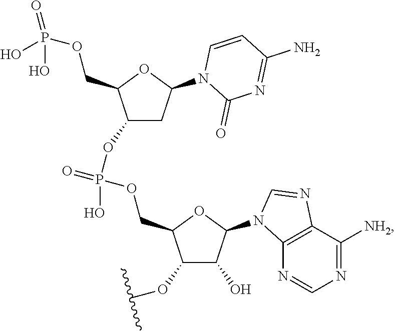 [SMILES: *O[C@H]1[C@@H](O)[C@H](n2cnc3c(N)ncnc32)O[C@@H]1COP(=O)(O)O[C@H]1C[C@H](n2ccc(N)nc2=O)O[C@@H]1COP(=O)(O)O]

In certain embodiments, Z is of the formula:

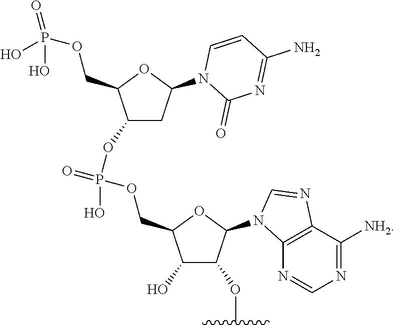 [SMILES: *O[C@@H]1[C@H](O)[C@@H](COP(=O)(O)O[C@H]2C[C@H](n3ccc(N)nc3=O)O[C@@H]2COP(=O)(O)O)O[C@H]1n1cnc2c(N)ncnc21]

In certain embodiments, the method comprises:
(a) a step of reacting a compound of Formula (II):

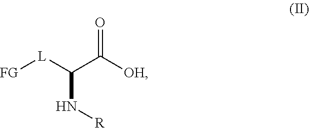

or a salt, stereoisomer, or tautomer thereof, with carbonyldiimidazole (CDI) to form a compound of Formula (A′):

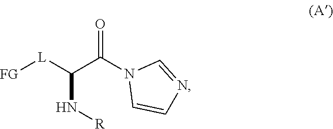

or a salt, stereoisomer, or tautomer thereof; and
(b) a step of reacting the compound of Formula (A′), or a salt, stereoisomer, or tautomer thereof, with the nucleotide. In certain embodiments, step (b) is carried out in a solvent comprising water.

In certain embodiments of the methods provided herein, the reaction is carried out in a solvent comprising water. In certain embodiments, the solvent comprising water comprises a mixture of water and a second solvent. In certain embodiments, the second solvent is DMF. In certain embodiments, solvent comprising water is a mixture of DMF and water.

In another aspect, provided herein are new fluorogenic sensors that in certain embodiments have increased sensitivity to Omicron variants of the SARS-CoV-2 virus. In certain embodiments, the fluorogenic sensors are based on any one of SEQ ID NOs: 1-4 (infra). In certain embodiments, the fluorogenic sensors are based on any one of SEQ ID NOs: 7-10 (infra).

Also provided herein are new fluorogenic sensors based on the sequence of EgA1 capable of binding and detecting EGFR proteins. The nanobody EgA1 specifically binds the human epidermal growth factor receptor (EGFR). In certain embodiments, the nanobody comprises an EgA1 nanobody or a fragment thereof. In certain embodiments, the fluorogenic sensors are based on SEQ ID NO: 5 or 6 (infra). Fluorogenic sensors for detecting other targets, such as cortisol and ALFA protein, are also provided.

Also provided herein are methods of detecting targets (e.g., SARS-CoV-2 variants, EGFR, cortisol, ALFA protein) with a fluorogenic sensor provided herein.

The details of certain embodiments of the invention are set forth in the Detailed Description of Certain Embodiments, as described below. Other features, objects, and advantages of the invention will be apparent from the Definitions, Examples, Figures, and Claims.

### Definitions

### General Definitions

The following definitions are general terms used throughout the present application.

The term “fluorogenic sensor” refers to a target-binding molecule (e.g., a protein, e.g., a nanobody or mini-protein) comprising a fluorogenic small molecule, that can be used to detect binding of the target-binding molecule to the target (e.g., to detect the presence of said target). The target-binding molecule may specifically bind the target. Upon binding of the target-binding molecule to the target, the fluorescence of the fluorogenic small molecule may increase or decrease, thereby “sensing” the target. I addition or alternatively, he fluorescence lifetime of the fluorogenic sensor may detectably change. In other words, an increase/decrease in fluorescence of the fluorogenic sensor or change in fluorescence lifetime of the sensor is indicative of binding of the target-binding molecule to the target, and therefore indicative of the presence of the target. In certain embodiments, the target is an antigen.

The term “target” or “target molecule” are used interchangeably, and as used herein refer any molecule or molecular structure (e.g., protein, antigen, small molecule) which is capable of being bound by a protein. As described herein, in certain embodiments, the target is an antigen, which is capable of being bound by an antigen-binding molecule (e.g., antibody, nanobody, mini-protein). In certain embodiments, the target is a small molecule. In certain embodiments, the target is an EGFR protein. In certain embodiments, the target is cortisol (e.g., cortisol sulfate). In certain embodiments, the target is an ALFA-tag protein.

The term “antigen” is a molecule or molecular structure, such as may be present on the outside of a pathogen (e.g., virus), that can be bound by an antigen-specific protein (e.g., antibody or nanobody). Antigens most often comprise proteins, peptides, and polysaccharides. The presence of antigens in the body normally triggers an immune response and are thereafter targeted for binding by antibodies. Examples of antigens include viruses, e.g., spike proteins of coronaviruses and variants thereof, e.g., spike proteins of the SARS-CoV-2 virus and variants thereof.

The terms “protein,” “peptide,” and “polypeptide” are used interchangeably and refer to a polymer of amino acid residues linked together by peptide bonds. The terms refer to peptides, polypeptides, and proteins, of any size, structure, or function. Typically, a protein will be at least three amino acids long, or at least the length required by an amino acid sequence provided herein. A protein may refer to an individual peptide or a collection of proteins. Proteins provided herein can include natural amino acids and/or unnatural amino acids (i.e., compounds that do not occur in nature but that can be incorporated into a peptide chain) in any combination. A protein may be a fragment or modified version of a naturally occurring protein. A protein may be naturally occurring, recombinant, synthetic, or any combination of these.

The term “nanobody” (Nanobody®) refers to a single-domain antibody (“sdAb”). A single-domain antibody is an antibody fragment consisting of a single monomeric variable antibody domain. Like full antibodies, single-domain antibodies are able to bind selectively to specific antigens. In certain embodiments, a nanobody will have a molecular weight of 12-15 kDa, inclusive.

A “target-binding domain” of a protein (e.g., nanobody) is a segment of the protein responsible for binding a target molecule. For example, an “antigen-binding domain” of a protein (e.g., nanobody) is a segment of the protein responsible for binding an antigen. A binding domain may be a group of consecutive amino acids of the amino sequence of the protein. In some instances, a protein (e.g., nanobody) provided herein will comprise more than one (e.g., 1, 2, 3) different binding domains.

The term “amino acid” refers to a molecule containing both an amino group and a carboxyl group. Amino acids include alpha-amino acids and beta-amino acids, the structures of which are depicted below. In certain embodiments, an amino acid is an alpha-amino acid. Each amino acid referred to herein may be denoted by a 1- to 4-letter code as commonly accepted in the art and/or as indicated below.

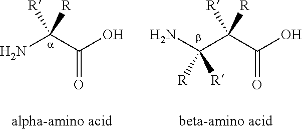

Suitable amino acids include, without limitation, natural alpha-amino acids such as D- and L-isomers of the 20 common naturally occurring alpha-amino acids found in peptides (e.g., A, R, N, C, D, Q, E, G, H, I, L, K, M, F, P, S, T, W, Y, V, as provided below), unnatural alpha-amino acids, natural beta-amino acids (e.g., beta-alanine), and unnatural beta-amino acids.

Exemplary natural alpha-amino acids (with one-letter code provided in parentheses) include L-alanine (A), L-arginine (R), L-asparagine (N), L-aspartic acid (D), L-cysteine (C), L-glutamic acid (E), L-glutamine (Q), glycine (G), L-histidine (H), L-isoleucine (I), L-leucine (L), L-lysine (K), L-methionine (M), L-phenylalanine (F), L-proline (P), L-serine (S), L-threonine (T), L-tryptophan (W), L-tyrosine (Y), and L-valine (V).

Exemplary unnatural alpha-amino acids include D-arginine, D-asparagine, D-aspartic acid, D-cysteine, D-glutamic acid, D-glutamine, D-histidine, D-isoleucine, D-leucine, D-lysine, D-methionine, D-phenylalanine, D-proline, D-serine, D-threonine, D-tryptophan, D-tyrosine, D-valine, Di-vinyl, α-methyl-alanine (Aib), α-methyl-arginine, α-methyl-asparagine, α-methyl-aspartic acid, α-methyl-cysteine, α-methyl-glutamic acid, α-methyl-glutamine, α-methyl-histidine, α-methyl-isoleucine, α-methyl-leucine, α-methyl-lysine, α-methyl-methionine, α-methyl-phenylalanine, α-methyl-proline, α-methyl-serine, α-methyl-threonine, α-methyl-tryptophan, α-methyl-tyrosine, α-methyl-valine, norleucine, and terminally unsaturated alpha-amino acids. There are many known unnatural amino acids any of which may be included in the peptides of the present disclosure. See for example, S. Hunt, The Non-Protein Amino Acids: In Chemistry and Biochemistry of the Amino Acids, edited by G. C. Barrett, Chapman and Hall, 1985. Unnatural amino acids also include amino acids comprising nitrogen substituents.

The term “amino acid substitution” when used in reference to an amino acid sequence refers to an amino acid of the amino acid sequence being replaced by a different amino acid (e.g., replaced by a natural or unnatural amino acid). An amino acid sequence provided herein may comprise or include one or more amino acid substitutions. Specific amino acid substitutions are denoted by commonly used colloquial nomenclature in the art of peptide sequencing to denote amino acid sequence variations. For example, the denotation “X #Y” refers to replacing the amino acid X at position # of the sequence with the amino acid Y. In certain embodiments, an amino acid sequence provided herein can comprise 0, 1, 2, 3, 4, 5, 6, 7, 8, 9, 10, 11, 12, 13, 14, 15, 16, 17, 18, 19, 20, 21, 22, 23, 24, or 25 amino acid substitutions. In certain embodiments, an amino acid of an amino acid sequence provided herein is substituted by a fluorogenic amino acid (FgAA).

The term “amino acid addition” when used in reference to an amino acid sequence refers to an amino acid (e.g., a natural or unnatural amino acid) being inserted between two amino acids of the amino acid sequence. In certain embodiments, an amino acid sequence herein can comprise 0, 1, 2, 3, 4, 5, 6, 7, 8, 9, 10, 11, 12, 13, 14, 15, 16, 17, 18, 19, 20, 21, 22, 23, 24, or 25 amino acid additions.

The term “amino acid deletion” when used in reference to an amino acid sequence refers to an amino acid of the amino acid sequence being deleted from the amino acid sequence. In certain embodiments, an amino acid sequence herein can comprise 0, 1, 2, 3, 4, 5, 6, 7, 8, 9, 10, 11, 12, 13, 14, 15, 16, 17, 18, 19, 20, 21, 22, 23, 24, or 25 amino acid deletions.

The term “fluorogenic small molecule” or “fluorophore” refers to a small molecule capable of emitting absorbed light, i.e., fluorescing. In certain embodiments, a fluorogenic small molecule can increase/decrease in fluorescence (i.e., “turn on”) in response to changes in viscosity, polarity, or other physical changes. In some embodiments, the fluorogenic small molecule exhibits a detectable change in fluorescence lifetime.

“Fluorescence” is the visible or invisible emission of light by a substance that has absorbed light or other electromagnetic radiation. It can be measured, e.g., by fluorescence microscopy. In certain embodiments, fluorescence is visible and can be detected by the naked eye. In certain embodiments, the detection is colorimetric.

Fluorophores such as the fluorogenic sensors provided herein have distinct fluorescence lifetime signatures, which can be detected, e.g., by a fluorescence lifetime microscopy. “Fluorescence lifetime” (FLT) is the time a fluorophore spends in the excited state before emitting a photon and returning to the ground state. Similar to fluorescence intensity, fluorogenic sensors also significantly change their fluorescence lifetimes based on the micro environment they are in. For example, when a viscosity sensor is free in solution and unconstrained, the sensor will be “darker” and typically will have a shorter fluorescence lifetime. On the other hand, when the sensor is physically restricted (e.g., in higher viscosity environments), they become brighter and show a signature, longer fluorescence lifetime.

The term “small molecule” refers to molecules, whether naturally-occurring or artificially created (e.g., via chemical synthesis) that have a relatively low molecular weight. Typically, a small molecule is an organic compound (e.g., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible.

As used herein, the term “conjugated” or “attached” when used with respect to two or more molecules, means that the molecules are physically associated or connected with one another, either directly (i.e., via a covalent bond) or via one or more additional moieties that serves as a linking agent (i.e., “linker”), to form a structure that is sufficiently stable so that the moieties remain physically associated under the conditions in which the structure is used, e.g., physiological conditions. For example, a fluorogenic small molecule provided herein can be “conjugated” to a protein by reacting a reactive moiety on the fluorogenic small molecule with an amino acid residue (e.g., lysine of cysteine residue) on the protein, thereby forming a covalent linkage between the protein amino acid and the fluorogenic small molecule. In certain embodiments, a fluorogenic small molecule is “conjugated” to a protein when a fluorogenic amino acid (FgAA) (i.e., an amino acid comprising a fluorogenic small molecule) is incorporated into the amino acid sequence of the protein.

As used herein, the term “salt” refers to any and all salts, and encompasses pharmaceutically acceptable salts. The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “tautomers” or “tautomeric” refers to two or more interconvertible compounds resulting from at least one migration of a hydrogen atom or electron lone pair, and at least one change in valency (e.g., a single bond to a double bond or vice versa). The exact ratio of the tautomers depends on several factors, including temperature, solvent, and pH. Exemplary tautomerizations include keto-to-enol, amide-to-imide, lactam-to-lactim, enamine-to-imine, and enamine-to-(a different enamine) tautomerizations. Compounds described herein are provided in any and all tautomeric forms. Example of tautomers resulting from the delocalization of electrons (e.g., resonance structures) are shown below:

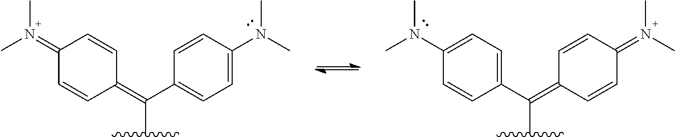

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. Biological samples may be derived from a nasal swab (e.g., nasopharyngeal swab) such as in the case of a SARS-CoV-2 or influenza test.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The disclosure additionally encompasses peptides as individual isomers substantially free of other isomers, and alternatively, as mixtures of various isomers.

In a formula, the bond [symbol] is a single bond, the dashed line [symbol] is a single bond or absent, and the bond [symbol] or [symbol] is a single or double bond. Additionally, the bond [symbol] or [symbol] is a double or triple bond.

Unless otherwise provided, formulae and structures depicted herein include peptides that do not include isotopically enriched atoms, and also include peptides that include isotopically enriched atoms (“isotopically labeled derivatives”). For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such peptides are useful, for example, as analytical tools or probes in biological assays. The term “isotopes” refers to variants of a particular chemical element such that, while all isotopes of a given element share the same number of protons in each atom of the element, those isotopes differ in the number of neutrons.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 6 carbon atoms (“C₁₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like.

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”).

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). The one or more carbon-carbon double bonds can be internal (such as in 2-butenyl) or terminal (such as in 1-butenyl). In an alkenyl group, a C═C double bond for which the stereochemistry is not specified (e.g., —CH═CHCH₃ or

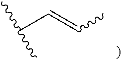

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”).

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). The one or more carbon-carbon triple bonds can be internal (such as in 2-butynyl) or terminal (such as in 1-butynyl).

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”).

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 6 ring carbon atoms (“C₃₋₆ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

The term “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur. In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom or the ring that does not contain a heteroatom.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A chemical moiety is optionally substituted unless expressly provided otherwise. Any chemical formula provided herein may also be optionally substituted. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, heteroaryl, acyl groups are optionally substituted. In general, the term “substituted” when referring to a chemical group means that at least one hydrogen present on the group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The disclosure is not limited in any manner by the exemplary substituents described herein.

Exemplary substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SCN, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring; and each X⁻ is a counterion.

In certain embodiments, each substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —N₃, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S—SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group. The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen. The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen. The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{aa}, —C(═O)OR^{aa}, —C(═O)—O—C(═O)R^{aa}, —C(═O)SR^{aa}, —C(═O)N(R^{bb})₂, —C(═S)R^{aa}, —C(═S)N(R^{bb})₂, and —C(═S)S(R^{aa}), —C(═NR^{bb})R^{aa}, —C(═NR^{bb})OR^{aa}, —C(═NR^{bb})SR^{aa}, and —C(═NR^{bb})N(R^{bb})₂, wherein R^{aa} and R^{bb} are as defined herein. Exemplary acyl groups include aldehydes (—CHO), carboxylic acids (—CO₂H), ketones, acyl halides, esters, amides, imines, carbonates, carbamates, and ureas.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

These and other exemplary substituents are described in more detail in the Detailed Description, Examples, Figures, and Claims. The disclosure is not limited in any manner by the above exemplary listing of substituents.

### BRIEF DESCRIPTION OF THE DRAWINGS

The accompanying drawings, which are incorporated in and constitute a part of this specification, illustrate several embodiments of the invention and together with the description, provide non-limiting examples of the invention.

FIG. 1. A comparison showing the preparation of fluorogenic sensors via chemical conjugation (Stage 1) vs. preparation of fluorogenic sensors via the evolution platform described herein (Stage 2).

FIG. 2. A two-staged nanosensor engineering platform enables rapid discovery, evolution, and cost-effective manufacturing of new optical biosensors. Stage 1: The target-bound protein binder crystal structure informs about the residues at/near the binding interface that can be substituted with lysines or cysteines to produce binder variants. Selected DNA sequences encoding protein binder variants are cloned, and variants are expressed and purified. Variants are conjugated to a library of thiol- and amine-reactive probes and nanosensor candidates are screened in a 384-well plate format. Stage 2: Lysine- or cysteine-probe conjugates that represent the ‘mature’ fluorogenic residues of nanosensors discovered in Stage 1 are synthesized as fluorogenic amino acids (FgAAs) and charged to an orthogonal tRNA_{CUA} via a pdCpA dinucleotide intermediate. The active nanosensors are produced ribosomally by site-specific incorporation of the FgAAs in vitro. The ability to produce nanosensors ribosomally enables directed evolution of the nanosensors by cDNA/mRNA display and selection. Enriched nanosensor variants can be screened ribosomally or produced in higher quantities via E. coli expression followed by chemical conjugation of the reactive probes.

FIG. 3. Ribosomal translation of fluorogenic amino acids via an efficient genetic code expansion chemistry enables in vitro directed evolution of nanosensors. (a) The optimized chemical synthesis of nsAA-pdCpA intermediate as compared to other methods. (b) Ribosomally constructed Wuhan-NS (via chemoenzymatically charged NBDxK-tRNA_{CUA}) can detect RBD_{W} in real time despite the presence of excess fluorogenic NBDxK-tRNA_{CUA} in the reaction. Lines and shaded areas represent the average and standard deviation of triplicate measurements. The ability to ribosomally produce nanosensors enables directed biosensor evolution via mRNA/cDNA. (c) New nanosensor sequences are enriched when selecting for the RBD_{OB.1} antigen. (d) Dose response curves with the evolved nanosensors (Omicron-NS-1/3) show their high affinity and fluorescence fold increases relative to Wuhan-NS when exposed to RBD_{OB.1}. (e) The biosensor discovery approach is generalizable to other protein scaffolds and molecular targets. Maximum fold increases of different sensors in the presence of saturating concentrations of their corresponding antigens over buffer controls: H11-NS for RBD_{W}, sdAb-NS for SARS-CoV-2 nucleocapsid, LCB3-NS for RBD_{W}, ALFA-NS for ALFA peptide, EGFR-NS for EGFR, Cortisol-NS for the small molecule, Cortisol. (f) ALFA-NS can be used for live-cell in situ imaging of ALFA-tag labeled Protein A in Staphylococcus aureus. Dots represent individual measurements. Lines represent a 4PL fit of the dose response curves. Shaded areas and dashed lines represent the 95% confidence intervals of the fits. Bars and error bars represent the average and standard deviation of triplicate experiments. Microscopy images are adjusted so that a quantitative comparison can be made. Scale bar=2 μm.

FIG. 4. Library design for the directed evolution of the Wuhan-NS. (a) In silico alignment of RBD_{OB.1} with the RBD_{W}-VHH72 complex shows potential steric clashes between RBD_{OB.1} at position F375 and VHH72 at position Y58. Steric clashes are not observed in the crystal structure of the complex VHH72-RBD_{W}. (b) The ˜10⁸ compensatory mutation library is built by modifying amino acids residues around a fixed fluorogenic amino acid position (V104NBDxK, here shown as 84TAG). CDR: Complementarity-determining regions.

FIG. 5. High Resolution mass spectrometry (MS) data for (a) BDPaF-pdCpA and (b) NBDxK-pdCpA, as compared to the respective theoretical isotropic patterns verified that the pdCpA-nsAA compounds can be accessed within a day with the herein optimized genetic code expansion chemistry. BDPaF-pdCpA: HRMS-ESI (m/z): Calc. for C₄₂H₅₀BF₂N₁₂O₁₅P₂ [M+H]⁺ 1073.3049, found 1073.3063. NBDxK-pdCpA: HRMS-ESI (m/z): Calc. for C₃₇H₅₁N₁₄O₁₈P₂ [M+H]⁺ 1041.2975, found 1041.2985.

FIG. 6. Fluorescence measured in gel electrophoresis shows that precursor tRNA(-CA)_{CUA} can be ligated to the BDPaF-pdCpA. (a) SYBR gold staining of the gel (b) in-gel fluorescence before SYBR gold staining. Gel bands: (1) molecular weight marker, (2) precursor PylT tRNA(-CA)_{CUA}, (3) precursor Mycoplasma capricolum Trp1 tRNA(-CA)_{CUA} (4) BDPaF-PyIT after ligation (5) BDPaF-McTrp1 after ligation. Ligation yield is calculated as the ratio of the fluorescence intensities in the SYBR gold staining gel image.

FIG. 7. Gel electrophoresis demonstrates the site specific ribosomal incorporation of BDPaF into different protein contexts. In-gel fluorescence of various proteins after site-specific incorporation of BDPaF. Lanes: (1) No DNA template control, (2) nanobody scaffold #1 (14.7 kDa) labelled at position 29, (3) nanobody scaffold #2 (12.4 kDa) labelled at position 27, (4) nanobody scaffold #2 (12.4 kDa) labelled at position 2, (5) nanobody scaffold #2-sfGFP fusion (40 kDa) labelled at position 27, and (6) lysozyme (19 kDa) labelled at position 158.

FIG. 8. Mass spectrometry (MS) was used to verify site-specific ribosomal incorporation of NBDxK into different positions of a polypeptide. NBDxK was ribosomally incorporated into two positions of the small peptide fMFPVFV, that can easily be detected by MS.

FIG. 9. NBDxK can be ribosomally incorporated into different protein contexts efficiently. (a) In-gel fluorescence and (b) Comassie staining of various proteins after site-specific incorporation of NBDxK via amber suppression in vitro using PURExpress® Δ RF123 Kit, NEB. Lanes: (1) NBDxK-PyIT no DNA template control, (2) Dihydrofolate reductase (DHFR, 21.6 kDa) labelled at position 2 via NBDxK-PyIT, (3) DHFR (21.6 kDa) labelled at position 155 via NBDxK-PyIT, (4) NBDxK-McTrp1 no DNA template control, (5) DHFR (21.6 kDa) labelled at position 2 via NBDxK-McTrp1, (6) DHFR (21.6 kDa) labelled at position 155 via NBDxK-McTrp1. Red arrow indicates efficient DHFR translation that is visible even in the Coomassie staining as it runs at a distinct position in the gel compared to the other PURE protein components. White arrow likely indicates tRNA species that remains fluorescently labeled at the end of the experiment.

FIG. 10. Nanobodies containing nsAAs can be evolved by mRNA/cDNA display. (1) A Wuhan-NS-based DNA library was designed that included an in-frame TAG at position 104, as well as mutations at eight other binding interface locations. The library was transcribed into mRNA and ligated to a 3′ puromycin-DNA linker with ˜50% efficiency. The nanobody library was then translated in the presence of the amber-decoding orthogonal fluorogenic amino acid (FgAA)-tRNA_{CUA} and covalently linked to its mRNA with ˜10% efficiency, as measured by in-gel fluorescence. (2) An Oligo dT25 purification step was used to remove unlinked nanobodies. Reverse transcription followed by Ni-NTA purification of the C-terminally His-tagged nanobodies recovers full-length nanosensors linked to their mRNA and cDNA. (3) mRNA-cDNA-nanobody complexes are first exposed to a ‘naked’ solid-phase matrix (e.g., magnetic beads) to remove non-specific binders (negative selection). The supernatant is then incubated with the same kind of beads containing the immobilized antigen of interest—in this case RBD_{OB.1}—(positive selection). Bound nanobodies are retained in the solid phase, washed, and eluted. PCR enables recovering enriched nanobody variants for the next rounds of selection. 7 rounds of selection were enough to enrich for nanobody variants that had a high affinity for RBD_{OB.1}.

FIG. 11. Directed evolution of nanosensor variants by selecting for binding to SARS-CoV-2 Omicron B.1.1.529 RBD (RBD_{OB.1}) resulted in the enrichment of new sequences. (a) Sanger sequencing of ˜150 individual colonies showed convergence to sequences that had mutations in CDR2, but CDR3—containing the unnatural fluorogenic amino acid-remained unchanged. The VHH72 residue predicted to clash with RBD_{OB.1} is shown in red (VHH72 Y58 from FIG. 4A) (b) Sequence frequency of Omicron-NS-1 and Omicron-NS-2 dramatically increase across the mRNA display rounds as measured by NGS.

FIG. 12. Rapid screening of ribosomally translated nanosensors without protein purification directly revealed new Omicron nanosensors. Crude, cell-free translation reactions of the nanosensors were mixed into microtubes containing PBS (negative control), RBD_{W}, or RBD_{OB.1}. Columns: Wuhan-NS, Omicron-1/3 and random library variants as negative controls (1, 2, 3, 4) The tubes were imaged and their fluorescence was quantified by ImageJ and normalized. Omicron nanosensors (Omicron-NS-1/3) increased fluorescence strongly in the presence of RBD_{W} and RBD_{OB.1}, while the Wuhan-NS only recognized RBD_{W}. No signal was observed in the negative controls.

FIG. 13. Dose response curve of the Wuhan nanosensor, VHH72 G56MDCcC, is like that of Wuhan-NS. Wuhan-NS detects RBD_{W} in both serum and in PBS. The Omicron-NS-1, selected by directed biosensor evolution, outperforms Wuhan-NS both in sensitivity and fold fluorescence increase.

FIG. 14. Biolayer interferometry to measure the affinities of evolved Omicron nanosensors for RBD_{OB.1}. Association and dissociation responses (a and c), and analysis using a steady state method (b, d) of the nanosensors Omicron-NS-1 (a-b) and Omicron-NS-2 (c-d). K_{D} values and fit parameters are included in Table 1.

FIG. 15. The Omicron nanosensors that resulted from the directed biosensor evolution pipeline selectively responded to some variants of the Omicron RBD. Dose response curves of (a) Omicron NS-1, (b) Omicron-NS-2 and (c) Omicron-NS-3 in the presence of SARS-CoV-2 variants RBD_{OB.1}, RBD_{OB.2}, RBD_{OB.3} and RBD_{OB-4.5}. Dots represent individual measurements. Lines represent a 4PL fit of the dose response curves. Shaded areas and dashed lines represent the 95% confidence intervals of the fits.

FIG. 16. Dose response curves of new nanosensors to their respective targets show that the platform can generate nanosensors against protein, peptide and small molecule targets. New nanosensors can be identified in three weeks. Fluorescence increase dose response curve of a) H11-H4 NoK R27DCcK (H11-NS) against RBD_{W}, b) sdAb-B6 NoK K65RhoRedxK (sdAb-NS) against the SARS-CoV-2 nucleocapsid protein c) LCB3 H19aNBDC (LCB3-NS) against RBD_{W}, d) NbALFA M63aNBDC (ALFA-NS) against the synthetic ALFA peptide, e) EgA1 S31MDCpcC (EGFR-NS) against human EGFR, and f) NbCor T53aNBDC (Cortisol-NS) against the small molecule cortisol sulfate. Dots represent individual measurements. Lines represent a 4PL fit of the dose response curves. Shaded areas and dashed lines represent the 95% confidence intervals of the fits.

FIG. 17. The modular nsAA-pdCpA diversification strategy enables ribosomal incorporation of diverse fluorogenic amino acids from new nanosensors. Fluorogenic amino acids (FgAAs) that are common ‘mature chromophores’ in biosensors in the literature and in nanosensors other than Wuhan-NS in this work were charged onto tRNA_{CUA} and translated into proteins within a week. These novel FgAAs included MDCcC (FgAA in biosensors such as phosphate binding protein based phosphate biosensor, or a T7 DNA polymerase-based DNA base pair biosensor and nanosensors like VHH72 G56MDCcC), aNBDC (FgAA in biosensors such as annexin-based apoptosis biosensor, designed ankyrin repeat proteins-based maltose binding protein biosensor or various periplasmic binding protein-based biosensors, and nanosensors like LCB3-NS, ALFA-NS, and Cortisol-NS), and DCcaK (the FgAA of sensors like H11-NS). See, e.g., Hirshberg, M. et al. “Crystal Structure of Phosphate Binding Protein Labeled with a Coumarin Fluorophore, a Probe for Inorganic Phosphate” Biochemistry 37, 10381-10385 (1998); Tsai, Y. C., Jin, Z. & Johnson, K. A. “Site-specific labeling of T7 DNA polymerase with a conformationally sensitive fluorophore and its use in detecting single-nucleotide polymorphisms” Analytical biochemistry 384, 136-144 (2009).; Kim, Y. E., Chen, J., Chan, J. R. & Langen, R. “Engineering a polarity-sensitive biosensor for time-lapse imaging of apoptotic processes and degeneration” Nature Methods 7, 67-73 (2010); Brient-Litzler, E., Pluckthun, A. & Bedouelle, H. “Knowledge-based design of reagentless fluorescent biosensors from a designed ankyrin repeat protein” Protein engineering, design & selection: PEDS 23, 229-241 (2010); and de Lorimier, R. M. et al. Construction of a fluorescent biosensor family. Protein Sci 11, 2655-2675 (2002). (a) Reaction scheme to access aNBDC-pdCpA and MDCcC-pdCpA through a modular pdCpA-cysteine-probe diversification strategy leveraging the selective reactivities of thiols with maleimides (top) or iodoacetamides (bottom). (b) In-gel fluorescence and (c) Comassie staining of Dihydrofolate reductase (DHFR, 21 kDa) and VHH72 (15 kDa) after site-specific incorporation of aNBDC and MDCcC via amber suppression using PURExpress® Δ RF123 Kit, NEB. Lanes: (1) DHFR labelled at position 2 via aNBDC-PyIT, (2) VHH72 labeled at position 104 via aNBDC-PyIT, (3) MDCcC-PyIT no DNA template control, (4) DHFR labelled at position 2 via MDCcC-PyIT, (5) VHH72 labeled at position 104 via MDCcC-PyIT. Red arrows indicate efficient DHFR translation that is visible in the Coomassie staining. Orange arrows indicate the location of labeled VHH72. (d) Reaction scheme to access DCcaK-pdCpA through Boc-DCcaK. (e) In-gel fluorescence and (f) Comassie staining of DHFR and VHH72 after site-specific incorporation of DCcaK via amber suppression using NEBExpress Cell-free E. coli Protein Synthesis System, NEB. DHFR labelled at position 2 (Lane 1) and VHH72 labeled at position 104 (Lane 2) via DCcaK-PyIT. White arrows indicate the locations of these labeled proteins.

FIG. 18. Structures of certain fluorogenic probes referenced throughout the disclosure.

### DETAILED DESCRIPTION OF CERTAIN EMBODIMENTS

A platform for preparing and employing fluorogenic sensors is described in International PCT Patent Application No. PCT/US2022/021878, published as WO 2022/204475 A1, the entire contents of which is incorporated herein by reference. The present disclosure relates, at least in part, to new fluorogenic sensors and new methods for preparing the same. For example, described herein is an evolution strategy that leverages highly efficient tRNA charging chemistry for cell-free ribosomal translation of proteins, including fluorogenic sensors. This evolution platform allows rapid molecular design of biosensors with applications in diagnostics, bio-surveillance, and molecular imaging.

Methods for Charging Nucleotides with Non-Standard Amino Acids (nsAAs)

The present disclosure in one aspect provides improved methods for chemically acylating nucleotides (e.g., pdCpA) with acyl moieties such as non-standard amino acids. The acylated (i.e., “charged”) nucleotides are building blocks of charged tRNA for use in the translation of proteins, such as the fluorogenic sensors provided herein.

In one aspect, provided herein are methods of selectively acylating pdCpA at the 2′-OH and/or 3′-OH positions comprising reacting pdCpA with an acylimidazole, wherein the step of reacting is carried out in a solvent comprising water. In certain embodiments, the reaction is selective for the 2′-OH position. In certain embodiments, the reaction is selective for the 3′-OH position.

In certain embodiments, provided herein is a method of selectively acylating pdCpA at the 2′-OH and/or 3′-OH positions to form the following:

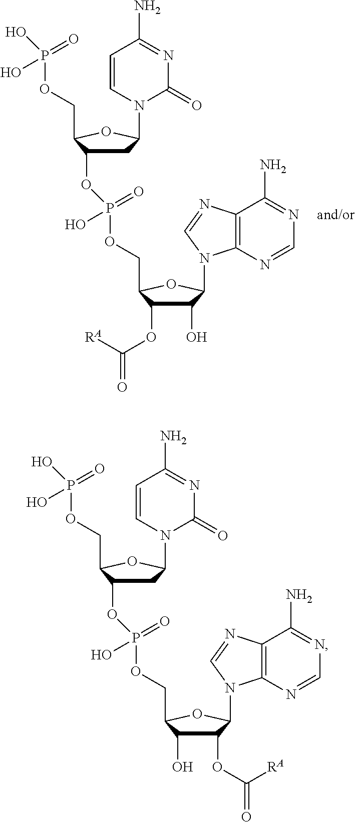

or salts thereof, the method comprising:
   (a) a step of reacting a compound of the formula: R^{A}(═O)OH, or a salt thereof, with carbonyldiimidazole (CDI) to form a compound of Formula (A):

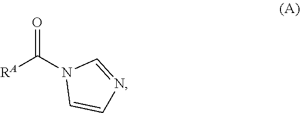

or a salt thereof; and
   (b) a step of reacting the compound of Formula (A), or a salt thereof, with pdCpA:

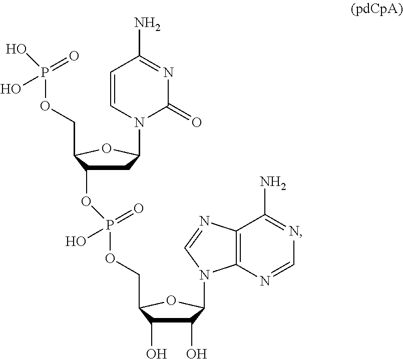

or a salt thereof,
   wherein step (b) of reacting is carried out in a solvent comprising water; and
   wherein R^{A} is an organic small molecule.

In certain embodiments, the group R^{A} is of the formula:

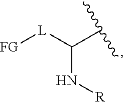

wherein:
   FG is a fluorogenic small molecule;
   L is a bond or a linker; and
   R is hydrogen or a nitrogen protecting group.

Also provided herein is a method of preparing a compound of Formula (I):

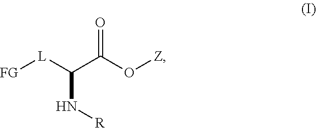

or a salt, stereoisomer, or tautomer thereof, wherein:
   FG is a fluorogenic small molecule;
   L is a bond or a linker;
   R is hydrogen or a nitrogen protecting group; and
   Z is a nucleotide;
comprising coupling a compound of Formula (II):

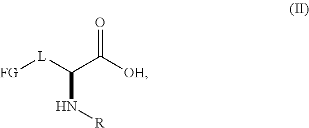

or a salt, stereoisomer, or tautomer thereof, with a nucleotide.

In certain embodiments, the compound of Formula (II) is coupled selectively at the 2′-OH and/or 3′-OH position of the nucleotide. In certain embodiments, Z is a mononucleotide, dinucleotide or polynucleotide. In certain embodiments, Z is a dinucleotide (e.g., pdCpA). In certain embodiments, Z is pdCpA.

In certain embodiments, Z is of the formula:

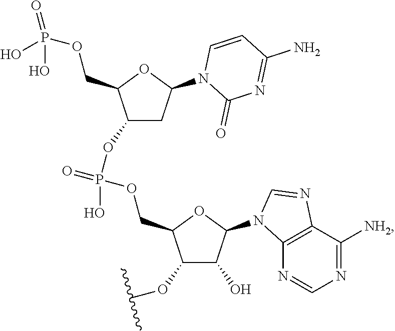 [SMILES: *O[C@H]1[C@@H](O)[C@H](n2cnc3c(N)ncnc32)O[C@@H]1COP(=O)(O)O[C@H]1C[C@H](n2ccc(N)nc2=O)O[C@@H]1COP(=O)(O)O]

In certain embodiments, Z is of the formula:

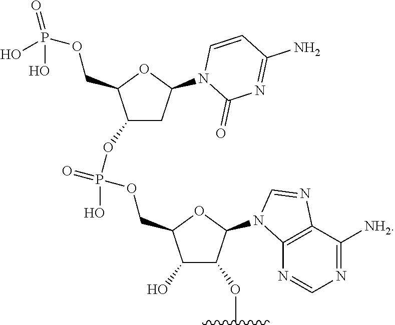 [SMILES: *O[C@@H]1[C@H](O)[C@@H](COP(=O)(O)O[C@H]2C[C@H](n3ccc(N)nc3=O)O[C@@H]2COP(=O)(O)O)O[C@H]1n1cnc2c(N)ncnc21]

In certain embodiments, the method comprises:
(a) a step of reacting a compound of Formula (II):

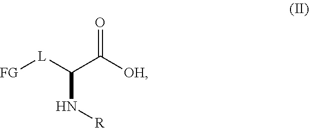

or a salt, stereoisomer, or tautomer thereof, with carbonyldiimidazole (CDI) to form a compound of Formula (A′):

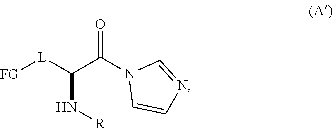

or a salt, stereoisomer, or tautomer thereof; and
   (b) a step of reacting the compound of Formula (A′), or a salt, stereoisomer, or tautomer thereof, with the nucleotide.

In certain embodiments of the methods provided herein, the reaction is carried out in a solvent comprising water. In certain embodiments, the solvent comprising water comprises a mixture of water and a second solvent. In certain embodiments, the second solvent is DMF. In certain embodiments, solvent comprising water is a mixture of DMF and water. In certain embodiments, the ratio of DMF:water is from 30:70 to 60:40 by volume. In certain embodiments, the ratio of DMF:water is from 40:60 to 50:50 by volume. In certain embodiments, the ratio of DMF:water is about 45:55 by volume.

In certain embodiments, the solvent has a pH of greater than 7. In certain embodiments, the solvent has a pH of greater than 7.5. In certain embodiments, the solvent has a pH of greater than 8. In certain embodiments, the solvent has a pH of greater than 9. In certain embodiments, the solvent has a pH of greater than 10. In certain embodiments, the solvent has a pH of about 7 to about 10. In certain embodiments, the solvent has a pH of about 7 to about 9. In certain embodiments, the solvent has a pH of about 8 to about 9. In certain embodiments, the solvent has a pH of about 7.5 to about 8.5. In certain embodiments, the solvent has a pH of about 8. In certain embodiments, the solvent has a pH of about 8.3.

In certain embodiments, the method further comprises a step of deprotecting a compound of Formula (I):

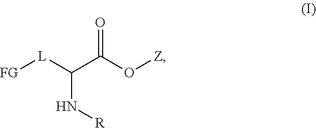

or a salt, stereoisomer, or tautomer thereof, wherein:
   FG is a fluorogenic small molecule;
   L is a bond or a linker;
   R is a nitrogen protecting group; and
   Z is a nucleotide;
to yield a compound of Formula (III):

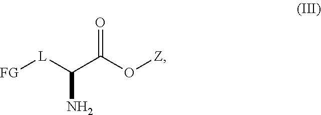

or a salt, stereoisomer, or tautomer thereof.

In certain embodiments, the step of deprotecting is carried out in the presence of an acid. In certain embodiments, the acid is an organic acid. In certain embodiments, the acid is a carboxylic acid. In certain embodiments, the acid trifluoroacetic acid. In certain embodiments, the acid is an inorganic acid.

In certain embodiments, the compounds disclosed herein contain the substituent R. In certain embodiments, R is hydrogen. In certain embodiments, R is a nitrogen protecting group. In certain embodiments, R is a carbamate protecting group. In certain embodiments, R is a tert-Butyloxycarbonyl (Boc) protecting group.

In certain embodiments, compounds provided herein comprise the group FG (e.g., a fluorogenic small molecule). In certain embodiments, FG comprises one of the following formulae:

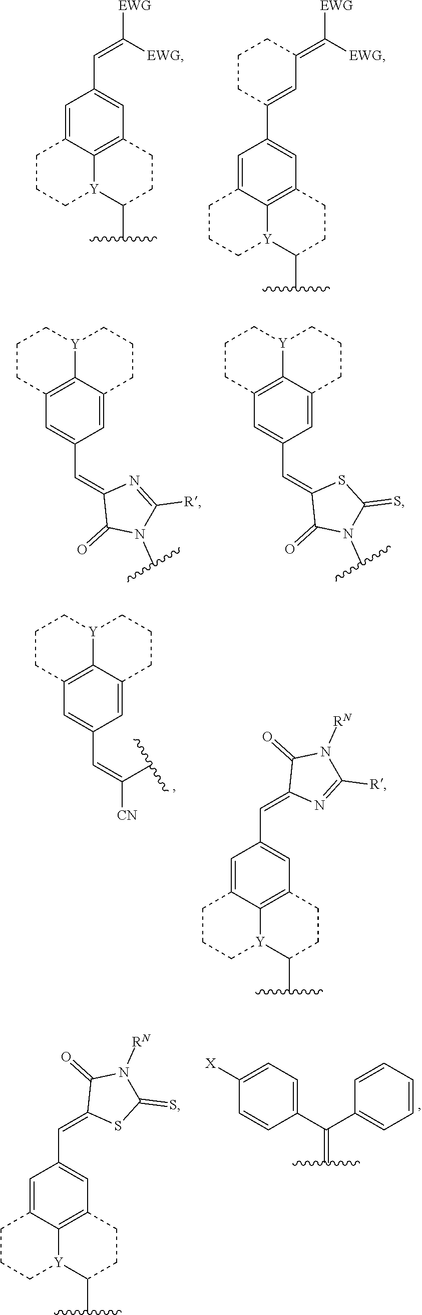

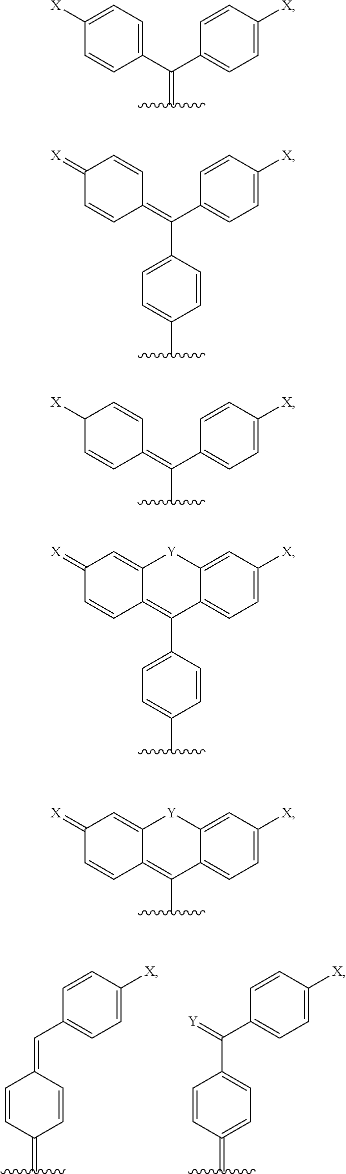

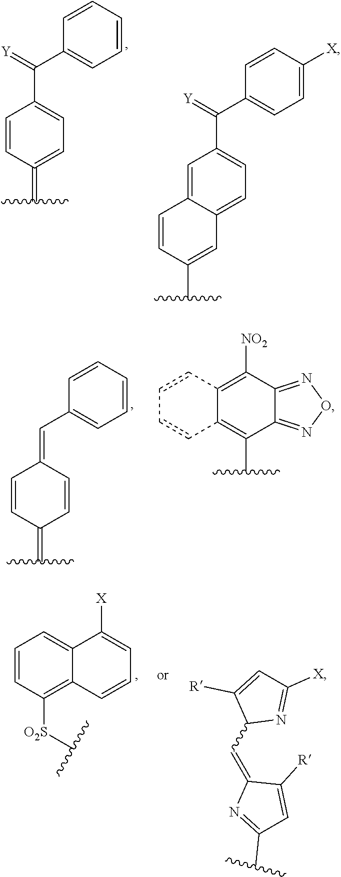

or a salt, stereoisomer, or tautomer thereof; wherein:
each instance of EWG is independently an electron withdrawing group;
Y is N, —NR^{N}, O, S, or —C(R′)₂;
each instance of X is independently —N(R^{N})₂, —OR^{O}, or —SR^{S};
each instance of R′ is independently hydrogen, halogen, —CN, —NO₂, —N₃, —N(R^{N})₂, —OR^{O}, —SR^{S}, alkyl, alkenyl, alkynyl, carbocyclyl, aryl, heterocyclyl, heteroaryl, acyl, sulfinyl, or sulfonyl; and
each instance of R^{N}, R^{O}, and R^{S} is independently hydrogen, alkyl, alkenyl, alkynyl, carbocyclyl, aryl, heterocyclyl, heteroaryl, or acyl; and
wherein each formula is further optionally substituted.

In certain embodiments, FG comprises one of the following:

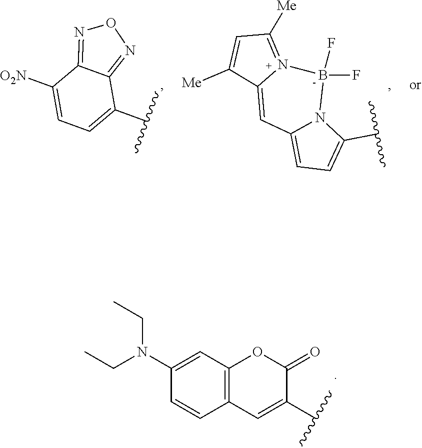

In certain embodiments, FG comprises one of the following:

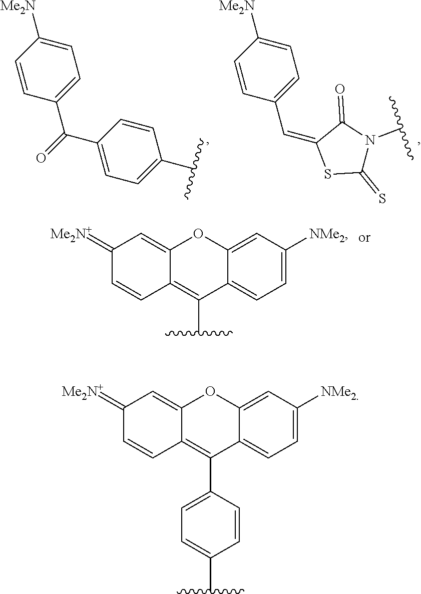

As also described herein, -L- is a bond or a linker. In certain embodiments, -L- is a bond. In certain embodiments, -L- is a linker of one of the following formulae:

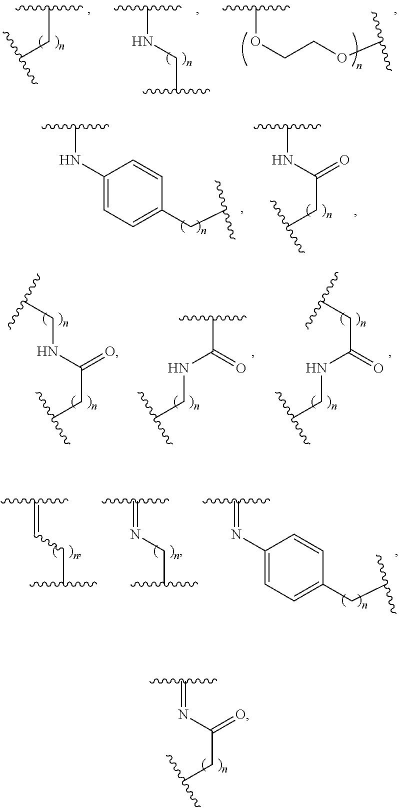

or a salt, stereoisomer, or tautomer thereof; wherein:
each n is independently 0 or an integer from 1-20, inclusive; and
wherein each formula is further optionally substituted.

In any of the methods provided herein, in certain embodiments, the compound of Formula (II) is one of the following:

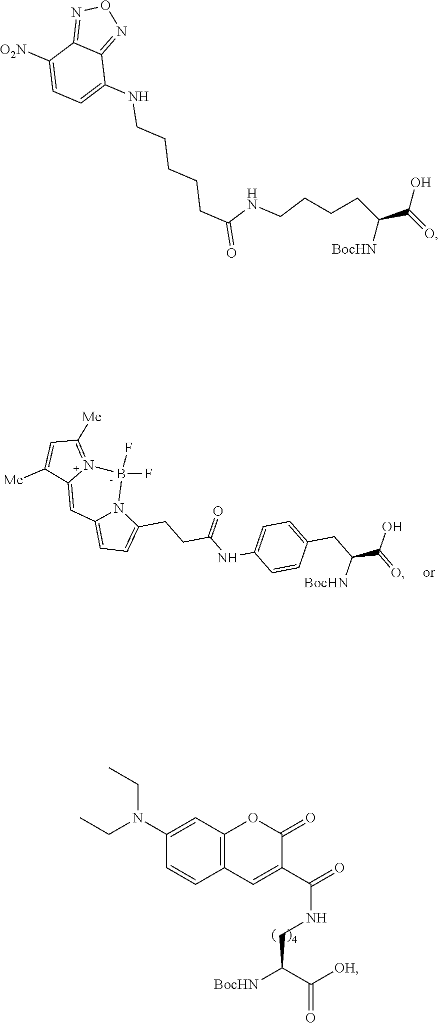

or a salt, stereoisomer, or tautomer thereof.

In any of the methods provided herein, in certain embodiments, the compound of Formula (II) is one of the following:

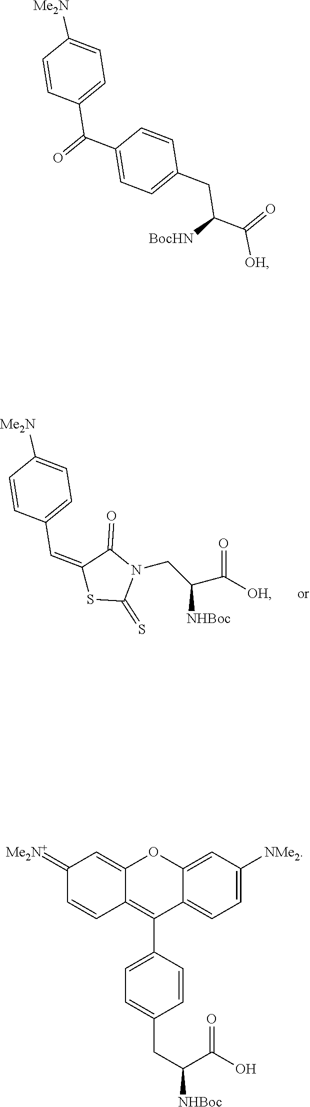

or a salt, stereoisomer, or tautomer thereof.

In any of the methods provided herein, in certain embodiments, the compound of Formula (I) is one of the following:

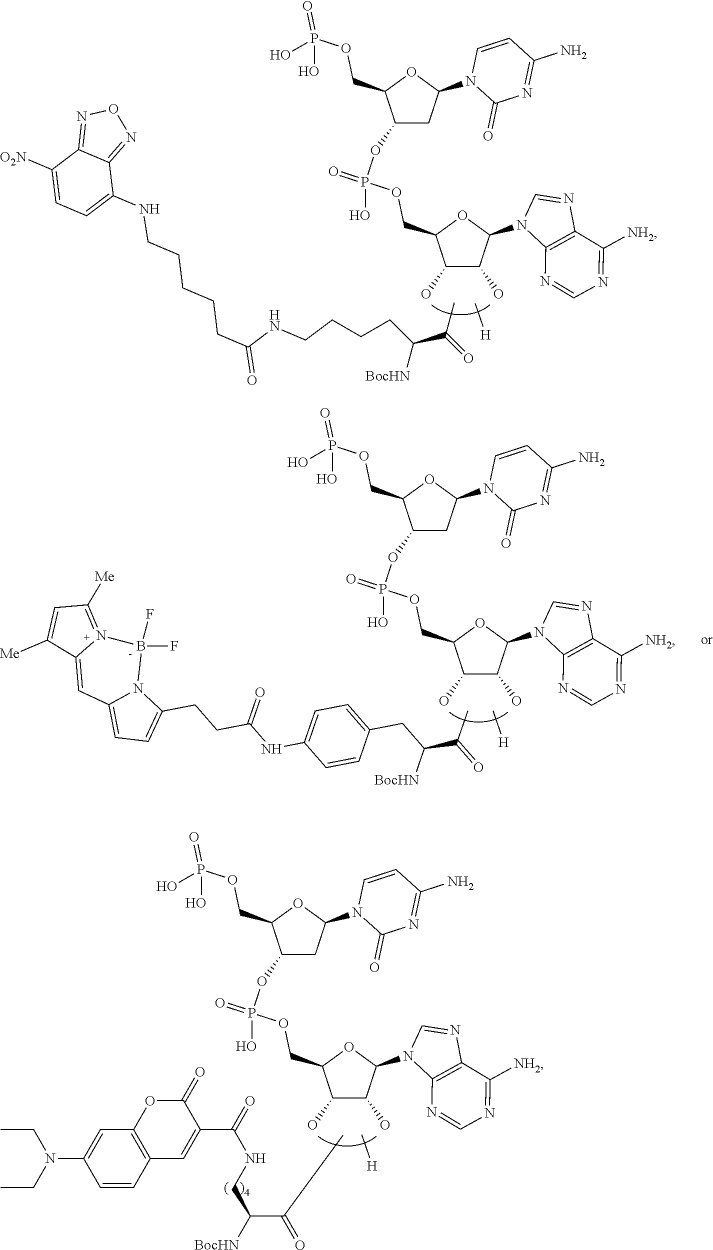

or a salt, stereoisomer, or tautomer thereof.

In any of the methods provided herein, in certain embodiments, the compound of Formula (I) is one of the following:

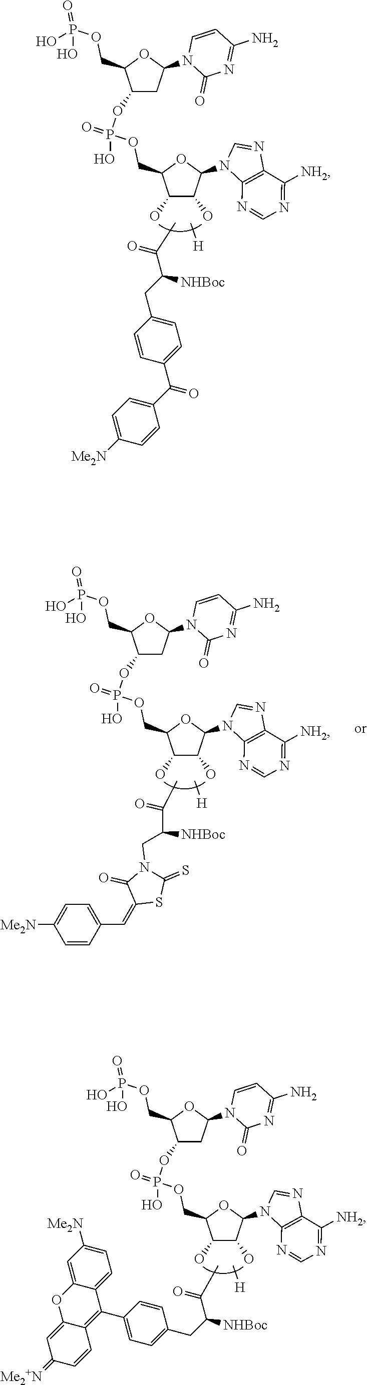

or a salt, stereoisomer, or tautomer thereof.

In any of the methods provided herein, in certain embodiments, the compound of Formula (III) is one of the following:

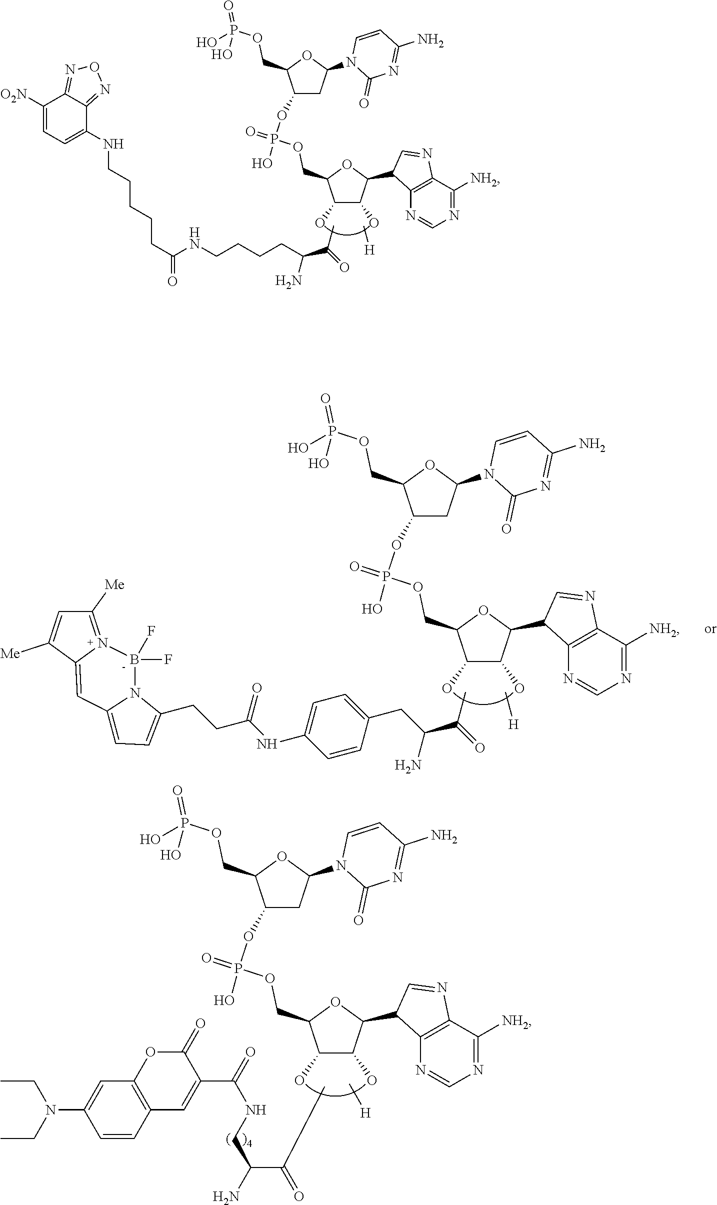

or a salt, stereoisomer, or tautomer thereof.

In any of the methods provided herein, in certain embodiments, the compound of Formula (III) is one of the following:

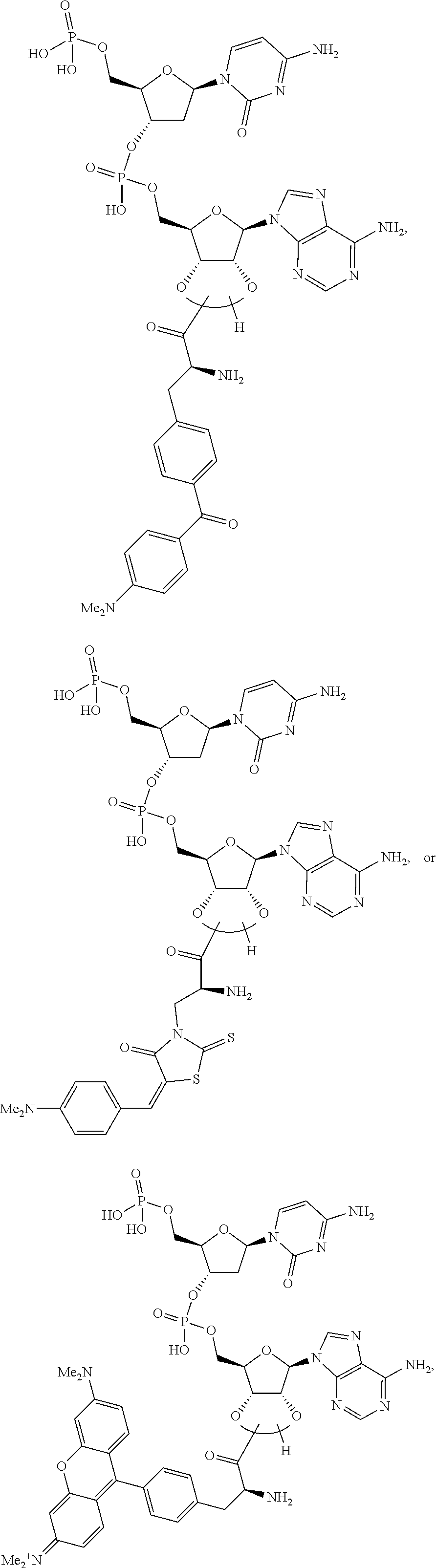

or a salt, stereoisomer, or tautomer thereof.

### Fluorogenic Sensors of SARS-CoV-2 Variants

Fluorogenic sensors of the SARS-CoV-2 spike protein are described in International PCT Patent Application No. PCT/US2022/021878, published as WO 2022/204475 A1, the entire contents of which is incorporated herein by reference. Provided herein are new fluorogenic sensors that in certain embodiments have increased sensitivity to Omicron variants of the SARS-CoV-2 virus.

Provided herein are fluorogenic sensors for detecting a target comprising a nanobody, wherein the nanobody comprises an amino acid sequence with at least 90% sequence identity with any one of SEQ ID NOs: 1-3:

(SEQ ID NO: 1)
QVQLQESGGGLVQAGGSLRLSCAASGRTFSEYAMGWFRQAPGREREFV
ATIGPSGGVTGYTDSVRGRFTISRDNARNTVYLQMNSLRPDDTAVYYC
AAAGLGTXVSEWDYDYDYWGQGTQVTVSSGSGGGSGGSGGGSG;
(SEQ ID NO: 2)
QVQLQESGGGLVQAGGSLRLSCAASGRTFSEYAMGWFRQAPGREREFV
ATIGPSGGITGYTDSVRGRFTISRDNARNTVYLQMNSLRPDDTAVYYC
AAAGLGTXVSEWDYDYDYWGQGTQVTVSSGSGGGSGGSGGGSG;
(SEQ ID NO: 3)
QVQLQESGGGLVQAGGSLRLSCAASGRTFSEYAMGWFRQAPGREREFV
ATILRSGGSTFYTDSVRGRFTISRDNARNTVYLQMNSLRPDDTAVYYC
AAAGLGTXVSEWDYDYDYWGRGTQVTVSSGSGGGSGGSGGGSG;

wherein X is a fluorogenic amino acid or amino acid conjugated to a fluorogenic small molecule.

In certain embodiments, the nanobody comprises an amino acid sequence at least 91%, 92%, 93%, 94%, 95%, 96%, 97%, 98%, or 99% identical to any one of SEQ ID NOs: 1-3. In certain embodiments, the nanobody comprises an amino acid sequence with 100% sequence identity with any one of SEQ ID NOs: 1-3. In certain embodiments, the nanobody binds (e.g., specifically binds) a spike protein of a coronavirus or variant thereof. In certain embodiments, the nanobody binds (e.g., specifically binds) a spike protein of a SARS-CoV-2 virus or variant thereof. In certain embodiments, the nanobody binds (e.g., specifically binds) a spike protein of an Omicron variant of SARS-CoV-2. Specific Omicron variants are described herein.

Also provided herein are fluorogenic sensors for detecting a target comprising a nanobody, wherein the nanobody comprises an amino acid sequence with at least 90% sequence identity with SEQ ID NO: 4:

(SEQ ID NO: 4)
QVQLVESGGGLMQAGGSLRLSCAVSGXTFSTAAMGWFRQAPGREREFVA
AIRWSGGSAYYADSVRGRFTISRDRARNTVYLQMNSLRYEDTAVYYCAQ
THYVSYLLSDYATWPYDYWGQGTQVTVSSGSGGSGGGSGGGSG;

wherein X is a fluorogenic amino acid or an amino acid conjugated to a fluorogenic small molecule.

In certain embodiments, the nanobody comprises an amino acid sequence at least 91%, 92%, 93%, 94%, 95%, 96%, 97%, 98%, or 99% identical to SEQ ID NO: 4. In certain embodiments, the nanobody comprises an amino acid sequence with 100% sequence identity with SEQ ID NO: 4. In certain embodiments, the nanobody binds (e.g., specifically binds) a spike protein of a coronavirus or variant thereof. In certain embodiments, the nanobody binds (e.g., specifically binds) a spike protein of a SARS-CoV-2 virus or variant thereof. In certain embodiments, the nanobody binds (e.g., specifically binds) a spike protein of an Omicron variant of SARS-CoV-2. Specific Omicron variants are described herein.

Also provided herein are fluorogenic sensors for detecting a target comprising a nanobody, wherein the nanobody comprises an amino acid sequence with at least 90% sequence identity with any one of SEQ ID NOs: 7-10:

(SEQ ID NO: 7)
MQVQLQESGGGLVQAGGSLRLSCAASGRTFSEXAMGWFRQAPGREREFV
ATISWSGGSTYYTDSVRGRFTISRDNARNTVYLQMNSLRPDDTAVYYCA
AAGLGTVVSEWDYDYDYWGQGTQVTVSSGSGGGSGGSGGGSG;
(SEQ ID NO: 8)
MQVQLQESGGGLVQAGGSLRLSCAASGRTFSEYAMGWFRQAPGREREFV
ATISWSGGXTYYTDSVRGRFTISRDNARNTVYLQMNSLRPDDTAVYYCA
AAGLGTVVSEWDYDYDYWGQGTQVTVSSGSGGGSGGSGGGSG;
(SEQ ID NO: 9)
MKIEEQVQLQESGGGLVQAGGSLRLSCAASGRTFSEYAMGWFRQAPGKE
REFVATIGPSGGCTGYTDSVKGRFTISRDNAKNTVYLQMNSLKPDDTAV
YYCAAAGLGTXVSEWDYDYDYWGQGTQVTVSSGSGGGSGGSGGGSG;
(SEQ ID NO: 10)
MKIEEQVQLQESGGGLVQAGGSLRLSCAASGRTFSEYAMGWFRQAPGKE
REFVATIGPSGGITYYTDSVKGRFTISRDNAKNTVYLQMNSLKPDDTAV
YYCAAAGLGVXISEWDYDYDYWGQGTQVTVSSGSGGGSGGSGGGSG;

wherein X is a fluorogenic amino acid or amino acid conjugated to a fluorogenic small molecule.

In certain embodiments, the nanobody comprises an amino acid sequence at least 91%, 92%, 93%, 94%, 95%, 96%, 97%, 98%, or 99% identical to any one of SEQ ID NOs: 7-10. In certain embodiments, the nanobody comprises an amino acid sequence with 100% sequence identity with any one of SEQ ID NOs: 7-10. In certain embodiments, the nanobody binds (e.g., specifically binds) a spike protein of a coronavirus or variant thereof. In certain embodiments, the nanobody binds (e.g., specifically binds) a spike protein of a SARS-CoV-2 virus or variant thereof. In certain embodiments, the nanobody binds (e.g., specifically binds) a spike protein of an Omicron variant of SARS-CoV-2. Specific Omicron variants are described herein.

### Fluorogenic Sensors of EGFR

Provided herein are fluorogenic sensors based on the sequence of EgA1 capable of binding and detecting EGFR proteins. The wild-type nanobody EgA1 specifically binds the human epidermal growth factor receptor (EGFR). In certain embodiments, the nanobody comprises an EgA1 nanobody or a fragment thereof. In certain embodiments, the nanobody comprises an amino acid sequence with at least 80% sequence identity with the sequence of EgA1 nanobody or a fragment thereof. In certain embodiments, the nanobody comprises an amino acid sequence with at least 50%, 55%, 60%, 65%, 70%, 75%, 80%, 85%, 90%, 95%, 98%, 99%, or 100% sequence identity with the sequence of a EgA1 nanobody, or a fragment thereof. EgA1 nanobodies comprise SEQ ID NO: 5.

Provided herein are fluorogenic sensors for detecting a target comprising:
a nanobody that binds an epidermal growth factor receptor (EGFR); and
a fluorogenic small molecule conjugated to a target-binding domain of the nanobody.

In certain embodiments, the nanobody comprises an amino acid sequence with at least 80% sequence identity with SEQ ID NO: 5:

(SEQ ID NO: 5)
QVQLQESGGGLVQPGGSLRLSCAASGRTFSSYAMGWFRQAPGKQREFVA
AIRWSGGYTYYTDSVKGRFTISRDNAKTTVYLQMNSLKPEDTAVYYCAA
TYLSSDYSRYALPQRPLDYDYWGQGTQVTVSSGSGGSGGGSGGGSG.

In certain embodiments, the nanobody comprises an amino acid sequence with at least 80% sequence identity with SEQ ID NO: 6 (EgA1 S31X):

(SEQ ID NO: 6)
QVQLQESGGGLVQPGGSLRLSCAASGRTFSXYAMGWFRQAPGKQREFVA
AIRWSGGYTYYTDSVKGRFTISRDNAKTTVYLQMNSLKPEDTAVYYCAA
TYLSSDYSRYALPQRPLDYDYWGQGTQVTVSSGSGGSGGGSGGGSG;

wherein X is a fluorogenic amino acid or an amino acid conjugated to a fluorogenic small molecule.

In certain embodiments, the nanobody comprises an amino acid sequence at least 85%, 90%, 91%, 92%, 93%, 94%, 95%, 96%, 97%, 98%, or 99% identical to SEQ ID NO: 5 or 6. In certain embodiments, the nanobody comprises an amino acid sequence with 100% sequence identity with SEQ ID NO: 5 or 6.

### Fluorogenic Sensors of Cortisol

Provided herein are fluorogenic sensors based on the sequence of the nanobody NbCor capable of binding and detecting cortisol (e.g., cortisol sulfate). In certain embodiments, the nanobody comprises NbCor or a fragment thereof. In certain embodiments, the nanobody comprises an amino acid sequence with at least 80% sequence identity with the sequence of NbCor or a fragment thereof. In certain embodiments, the nanobody comprises an amino acid sequence with at least 50%, 55%, 60%, 65%, 70%, 75%, 80%, 85%, 90%, 95%, 98%, 99%, or 100% sequence identity with the sequence of NbCor, or a fragment thereof.

Provided herein are fluorogenic sensors for detecting a target comprising:
a nanobody that binds cortisol (e.g., cortisol sulfate); and
a fluorogenic small molecule conjugated to a target-binding domain of the nanobody.

In certain embodiments, the nanobody comprises an amino acid sequence with at least 80% sequence identity with SEQ ID NO: 11 (NbCor T53X):

(SEQ ID NO: 11)
QVQLQESGGGSVQAGGSLRLSCVVSGNTGSTGYWAWFRQGPGTEREGVA
AXYTAGSGTSMTYYADSVKGRFTISQDNAKKTLYLQMNSLKPEDTGMYR
CASTRFAGRWYRDSEYRAWGQGTQVTVSSGSGGSGGGSGGGSG

wherein X is a fluorogenic amino acid or an amino acid conjugated to a fluorogenic small molecule.

In certain embodiments, the nanobody comprises an amino acid sequence at least 85%, 90%, 91%, 92%, 93%, 94%, 95%, 96%, 97%, 98%, or 99% identical to SEQ ID NO: 11. In certain embodiments, the nanobody comprises an amino acid sequence with 100% sequence identity with SEQ ID NO: 11.

### Fluorogenic Sensors of ALFA Peptide

Also provided herein are fluorogenic sensors capable of binding and detecting synthetic ALFA peptide (i.e., ALFA-tag). In certain embodiments, the nanobody comprises NbALFA or a fragment thereof. In certain embodiments, the nanobody comprises an amino acid sequence with at least 80% sequence identity with the sequence of NbALFA or a fragment thereof. In certain embodiments, the nanobody comprises an amino acid sequence with at least 50%, 55%, 60%, 65%, 70%, 75%, 80%, 85%, 90%, 95%, 98%, 99%, or 100% sequence identity with the sequence of NbALFA, or a fragment thereof.

Provided herein are fluorogenic sensors for detecting a target comprising:
a nanobody that binds an ALFA peptide; and
a fluorogenic small molecule conjugated to a target-binding domain of the nanobody.

In certain embodiments, the nanobody comprises an amino acid sequence with at least 80% sequence identity with SEQ ID NO: 12 (NbALFA M63X):

(SEQ ID NO: 12)
QSSGQVQLQESGGGLVQPGGSLRLSCTASGVTISALNAMAMGWYRQAPG
ERRVMVAAVSERGNAXYRESVQGRFTVTRDFTNRMVSLQMDNLRPEDTA
VYYCHVLEDRVDSFHDYWGQGTQVTVSSGSGGSGGGSGGGSG.

wherein X is a fluorogenic amino acid or an amino acid conjugated to a fluorogenic small molecule.

In certain embodiments, the nanobody comprises an amino acid sequence at least 85%, 90%, 91%, 92%, 93%, 94%, 95%, 96%, 97%, 98%, or 99% identical to SEQ ID NO: 12. In certain embodiments, the nanobody comprises an amino acid sequence with 100% sequence identity with SEQ ID NO: 12.

### Fluorogenic Small Molecules

As described herein, the fluorogenic sensors comprise a fluorogenic small molecule at or around a target-binding domain (e.g., antigen-binding domain) of the protein (e.g., nanobody). The fluorogenic small molecule is conjugated or attached to the protein (e.g., nanobody or mini-protein) either through a covalent bond or linker moiety. In certain embodiments, the fluorogenic small molecule is part of a fluorogenic amino acid (FgAA).

In certain embodiments, the fluorogenic small molecule (e.g., FG) comprises one of the following formulae:

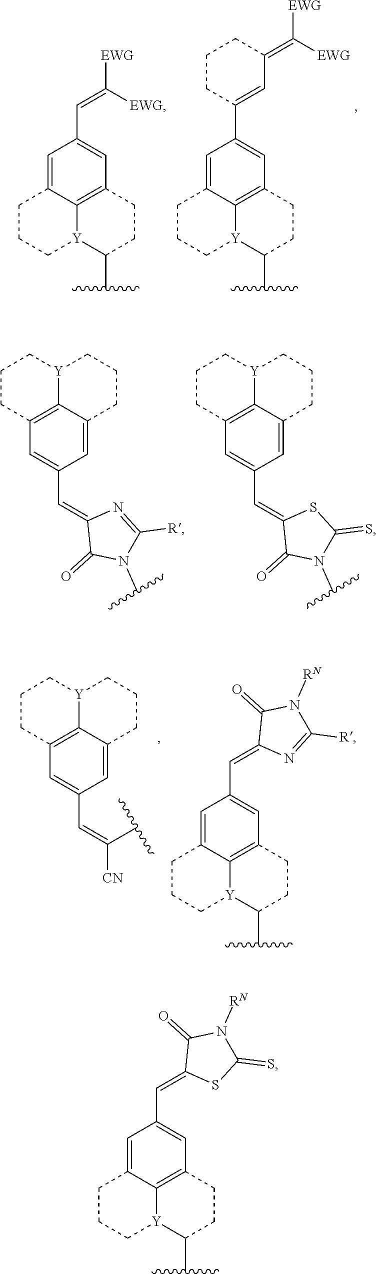

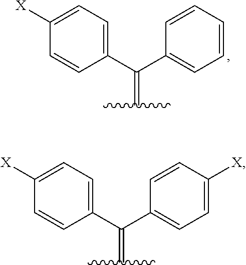

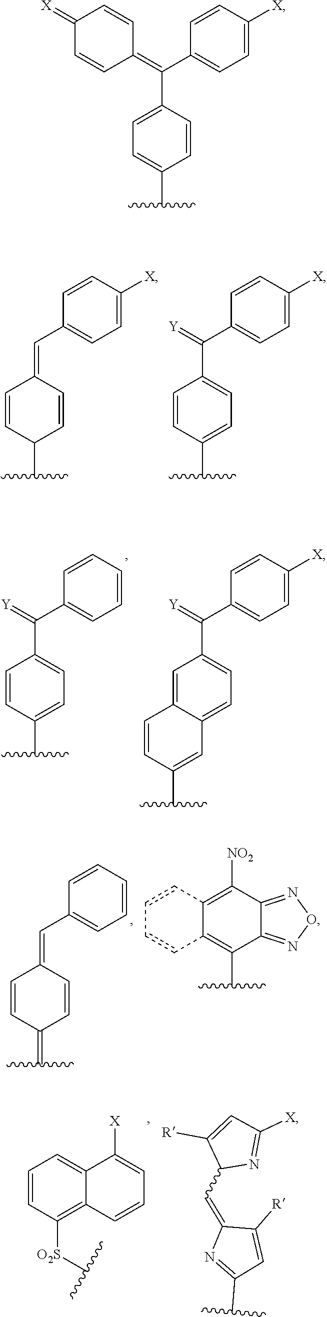

or a salt, stereoisomer, or tautomer thereof; wherein:
each instance of EWG is independently an electron withdrawing group; Y is N, —NR^{N}, O, S, or —C(R′)₂;
each instance of X is independently —N(R^{N})₂, —OR^{O}, or —SR^{S};
each instance of R′ is independently hydrogen, halogen, —CN, —NO₂, —N₃, —N(R^{N})₂, —OR^{O}, —SR^{S}, alkyl, alkenyl, alkynyl, carbocyclyl, aryl, heterocyclyl, heteroaryl, acyl, sulfinyl, or sulfonyl; and
each instance of R^{N}, R^{O}, and R^{S} is independently hydrogen, alkyl, alkenyl, alkynyl, carbocyclyl, aryl, heterocyclyl, heteroaryl, or acyl; and
wherein each formula is further optionally substituted.

Other examples of fluorogenic small molecules can be found in, e.g., Klymchenko et al. Acc. Chem. Res. 2017, 50, 366-375; the entire contents of which is incorporated herein by reference. Additional fluorogenic small molecules are provided in International PCT Patent Application No. PCT/US2022/021878, published as WO 2022/204475 A1, the entire contents of which is incorporated herein by reference.

In certain embodiments, FG comprises one of the following:

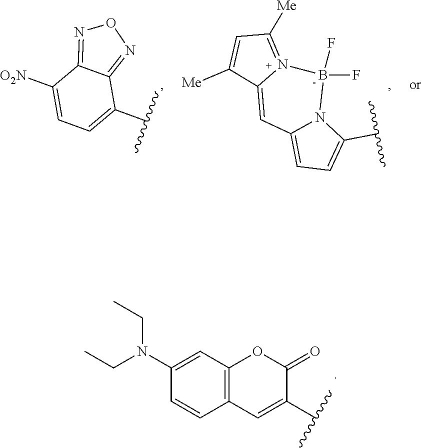

In certain embodiments, FG comprises one of the following:

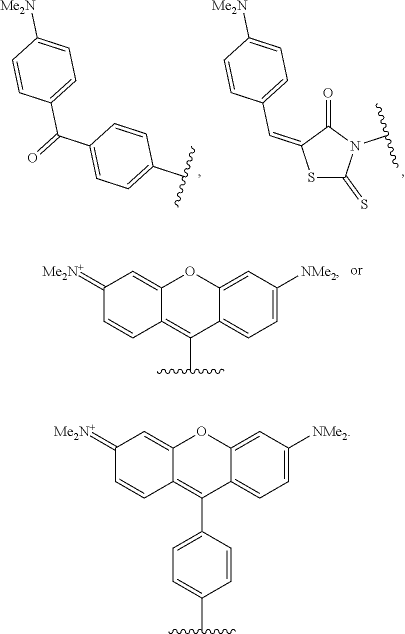

In certain embodiments, the fluorogenic small molecule conjugated to the protein (e.g., nanobody) results from conjugating a compound of the following formula (i.e., “fluorogenic probe”) to the protein:

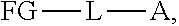

or a salt, stereoisomer, or tautomer thereof; wherein FG is the fluorogenic small molecule; L is a bond or a linker; and A is a reactive moiety.

In certain embodiments, the group -L-A is of one of the following formulae:

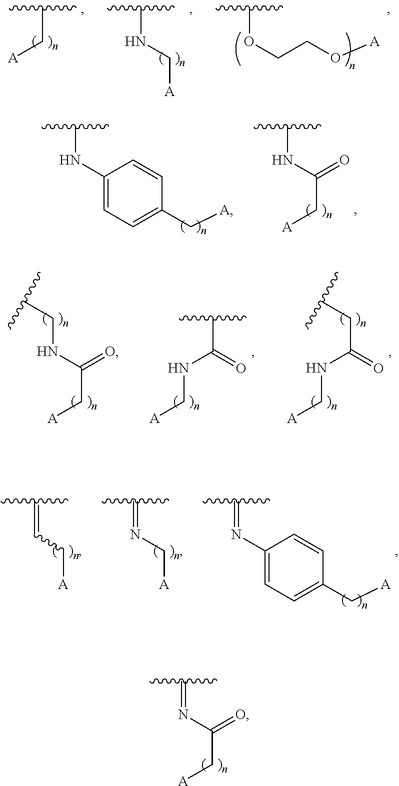

or a salt, stereoisomer, or tautomer thereof; wherein:
each n is independently 0 or an integer from 1-20, inclusive; and
wherein each formula is further optionally substituted.

As described herein, A is a reactive moiety. In certain embodiments, A is a lysine- or cysteine-selective reactive moiety. In certain embodiments A is a lysine-selective reactive moiety. In certain embodiments, A is a cysteine-selective reactive moiety.

For the purposes of this disclosure, a “reactive moiety” is any chemical moiety capable of reacting with another chemical moiety to form a covalent bond or covalent bonds. Non-limiting examples of reactive moieties include alkenes, alkynes, alcohols, amines, thiols, azides, esters, amides, halogens, and the like. In certain embodiments, two reactive moieties are capable of reacting directly with each other to form one or more covalent bonds. In other embodiments, two reactive moieties react with an intervening linking reagent to form a covalent linkage. In certain embodiments, the reactive moieties are click chemistry moieties. “Click chemistry” moieties are any moieties that can be used in click chemistry reactions.

“Click chemistry” is a chemical approach introduced by Sharpless in 2001 and describes chemistry tailored to generate substances quickly and reliably by joining small units together. See, e.g., Kolb, Finn and Sharpless Angewandte Chemie International Edition (2001) 40: 2004-2021; Evans, Australian Journal of Chemistry (2007) 60: 384-395. Exemplary coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition); and Diels-Alder reactions (e.g., tetrazine [4+2] cycloaddition). As an example, in the case of reactions between an azide and alkyne reactive moieties to form triazolylene linkages, alkyne-azide 1,3-cycloadditions may be used (e.g., the Huisgen alkyne-azide cycloaddition). In certain embodiments, the alkyne-azide cycloaddition is copper-catalyzed. In certain embodiments, the alkyne-azide cycloaddition is strain-promoted. Examples of alkyne-azide reactions can be found in, e.g., Kolb, Finn and Sharpless Angewandte Chemie International Edition (2001) 40: 2004-2021; Kolb and Sharpless, Drug Discov Today (2003) 24: 1128-1137; and Evans, Australian Journal of Chemistry (2007) 60: 384-395.

In certain embodiments, A comprises a halogen, alkene, alkyne, azide, tetrazine, or a moiety of one of the following formulae:

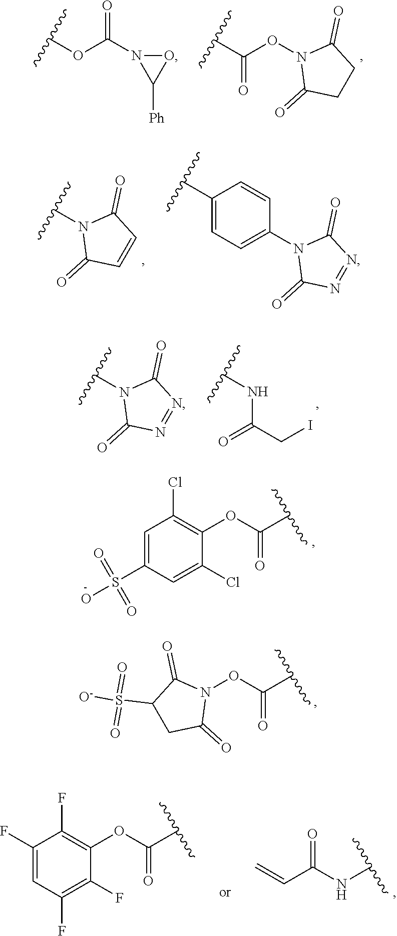

or a salt, stereoisomer, or tautomer thereof; wherein each formula is further optionally substituted.

The table below shows the reactive moieties and their associated chemoselectivity.

| Reactive Moiety | Chemoselectivity |
|---|---|
| azide | alkynes |
| alkyne | azides |
| 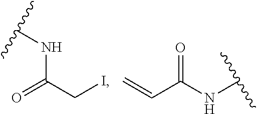 | thiols (e.g., cysteine) |
| 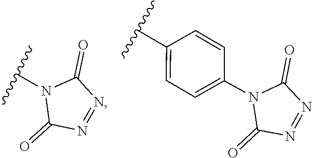 [SMILES: *N1C(=O)N=NC1=O ; *c1ccc(N2C(=O)N=NC2=O)cc1] | phenols (e.g., tyrosine) |
| 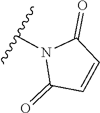 [SMILES: *N1C(=O)C=CC1=O] | thiols (e.g., cysteine) |
| 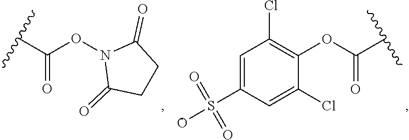 | amines (e.g., lysine) |
| 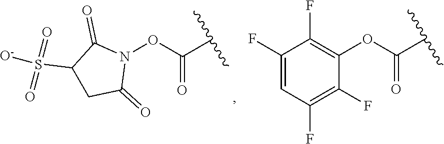 [SMILES: *C(=O)ON1C(=O)CC(S(=O)(=O)[O-])C1=O ; *C(=O)Oc1c(F)c(F)cc(F)c1F] | |
| 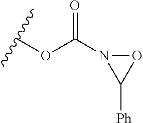 [SMILES: *OC(=O)N1OC1c1ccccc1] | thioethers (e.g., methionine) |

The present disclosure includes any of the foregoing fluorogenic probes (including any and all possible combinations of FG, L, and A) as part of the fluorogenic sensors described herein (i.e., conjugated to an antigen-binding protein (e.g., nanobody)), and also as compounds (i.e., not conjugated to a protein). Additional fluorogenic probes are provided in International PCT Patent Application No. PCT/US2022/021878, published as WO 2022/204475 A1, the entire contents of which is incorporated herein by reference.

### Fluorogenic Amino Acids

In some embodiments, a binding domain of the protein (e.g., nanobody) comprises an unnatural amino acid comprising a fluorogenic small molecule (i.e., “fluorogenic amino acid” or “FgAA”). In preferred embodiments, the fluorogenic small molecule is attached to the α-position of the FgAA (e.g., through a covalent bond or a linker moiety).

As described herein, in certain embodiments, tRNA is charged with a fluorogenic amino acid, e.g., via the nucleotide acylation methods descried herein (e.g., the pdCpA acylation methods described herein). tRNA charged with fluorogenic amino acids can be used to construct fluorogenic sensors comprising the fluorogenic amino acids ribosomally.

In certain embodiments, a fluorogenic amino acid comprises any one of the formulae provided for -FG (supra). Examples of fluorogenic amino acids which are considered part of the present disclosure can be found in, e.g., International PCT Application Publication WO 2021/118727, published Jun. 17, 2021, the entire contents of which is incorporated herein by reference. Additional fluorogenic amino acids are provided in International PCT Patent Application No. PCT/US2022/021878, published as WO 2022/204475 A1, the entire contents of which is incorporated herein by reference.

For example, in certain embodiments, the FgAA is of one of the following formulae:

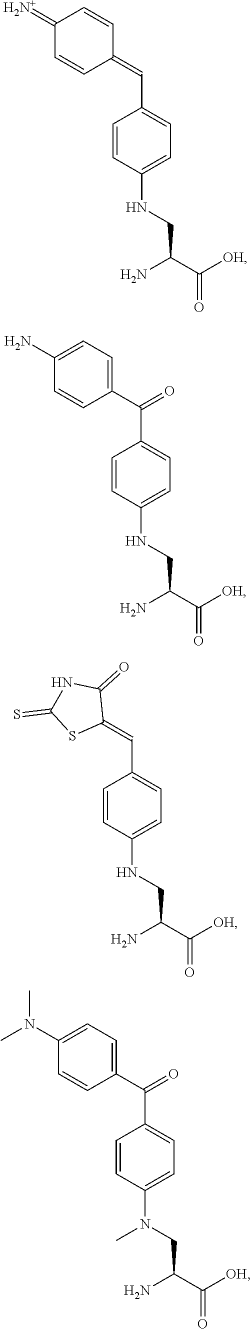 [SMILES: N[C@@H](CNc1ccc(C=C2C=CC(=[NH2+])C=C2)cc1)C(=O)O ; Nc1ccc(C(=O)c2ccc(NC[C@H](N)C(=O)O)cc2)cc1 ; N[C@@H](CNc1ccc(/C=C2\SC(=S)NC2=O)cc1)C(=O)O ; CN(C)c1ccc(C(=O)c2ccc(N(C)C[C@H](N)C(=O)O)cc2)cc1]

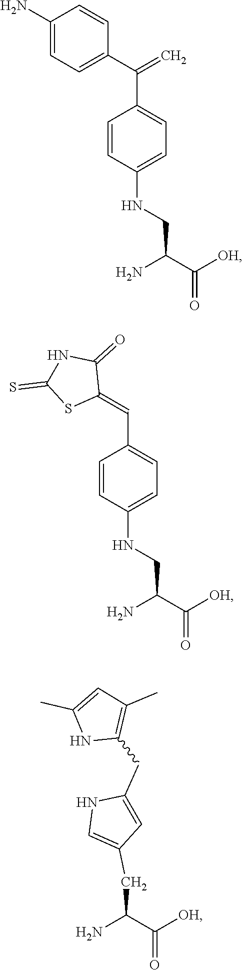 [SMILES: C=C(c1ccc(N)cc1)c1ccc(NC[C@H](N)C(=O)O)cc1 ; N[C@@H](CNc1ccc(/C=C2\SC(=S)NC2=O)cc1)C(=O)O ; Cc1cc(C)c(Cc2cc(C[C@H](N)C(=O)O)c[nH]2)[nH]1]

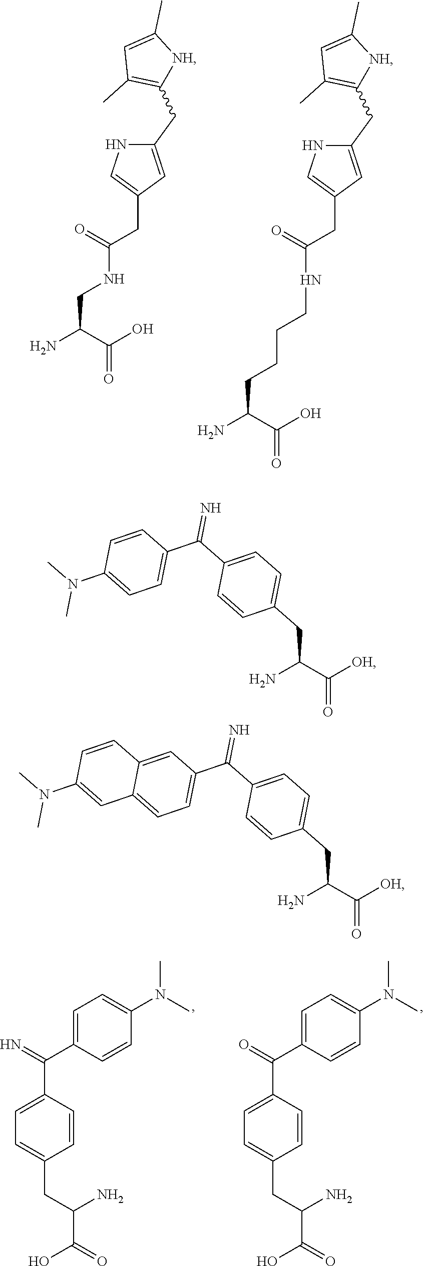

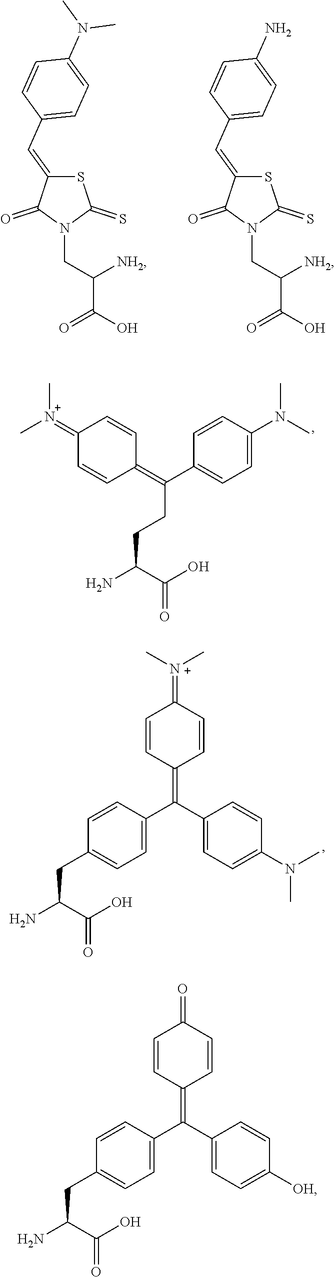

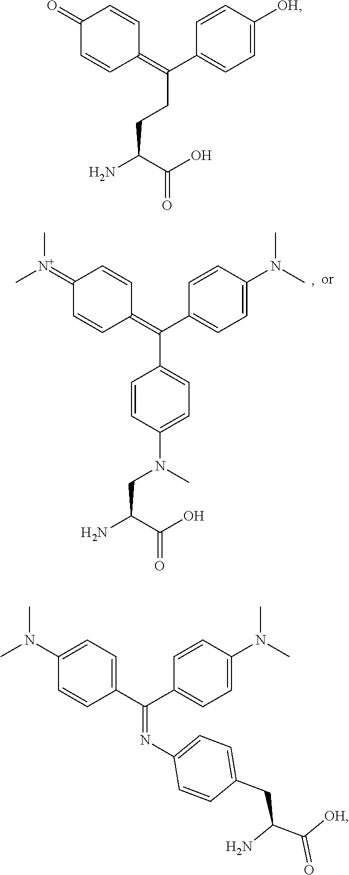

or a salt, stereoisomer, or tautomer thereof.

In certain embodiments, the fluorogenic amino acid is one of the following:

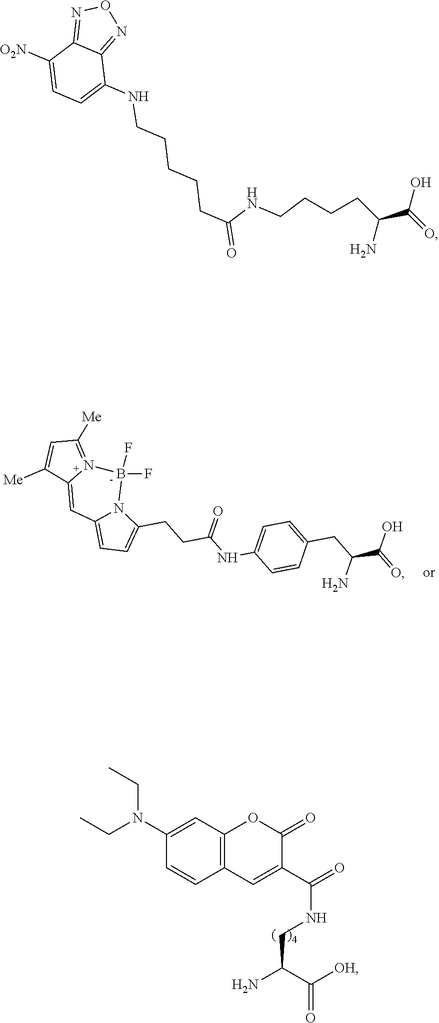

or a salt, stereoisomer, or tautomer thereof.

In certain embodiments, the fluorogenic amino acid is one of the following:

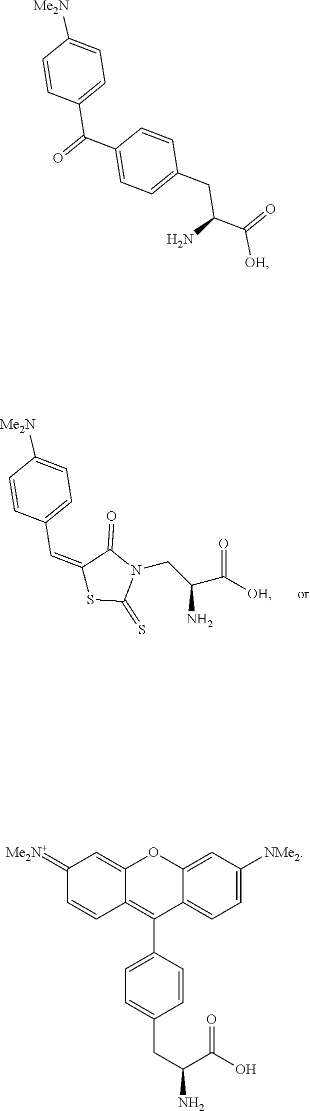

or a salt, stereoisomer, or tautomer thereof.

### Methods and Kits for Detecting Antigens

As described herein, the fluorogenic sensors can be used to detect protein-target interactions, and can therefore be used to detect the presence of a target (e.g., an antigen).

Provided herein are methods of determining the presence of target in a sample, the method comprising: (i) contacting a sample with a fluorogenic sensor provided herein; and (ii.a) measuring or observing the fluorescence of the sample or (ii.b) measuring or observing the change in fluorescence lifetime of the sample. As described herein, the fluorescence of the sample may increase upon binding of the fluorogenic sensor to the target. Therefore, any increase in fluorescence may be indicative of the presence of the target. In certain embodiments, the fluorescence lifetime of the sample may change upon binding of the fluorogenic sensor to the target.

As described herein, the fluorogenic sensors can be used to detect the presence of antigens. Provided herein are methods of determining the presence of an antigen in a sample, the method comprising: (i) contacting a sample with a fluorogenic sensor provided herein; and (ii.a) measuring or observing the fluorescence of the sample or (ii.b) measuring or observing the change in fluorescence lifetime of the sample. As described herein, the fluorescence of the sample may increase upon binding of the fluorogenic sensor to the antigen. Therefore, any increase in fluorescence may be indicative of the presence of the antigen. In certain embodiments, the fluorescence lifetime of the sample may change upon binding of the fluorogenic sensor to the target.

In certain embodiments, the fluorescence of the sample is increased by at least 10%. In certain embodiments, the fluorescence of the sample is increased by at least 10%, 20%, 30%, 40%, 50%, 60%, 70%, 80%, or 90%. In certain embodiments, the fluorescence of the sample is increased by at least 1-fold, 2-fold, 5-fold, 10-fold, 20-fold, 25-fold, 30-fold, 35-fold, 40-fold, 45-fold, 50-fold, 100-fold, 150-fold, 200-fold, 300-fold, 400-fold, or 500-fold. In certain embodiments, the increase in fluorescence is greater than 500-fold. In certain embodiments, the fluorescence of the sample is increased by at about 5- to about 25-fold. In certain embodiments, the fluorescence of the sample is increased by at about 5- to about 100-fold. In certain embodiments, the fluorescence of the sample is increased by at about 5- to about 50-fold. In certain embodiments, the fluorescence of the sample is increased by at least 100-fold.

In certain embodiments, the fluorescence of the sample is decreased by at least 10%. In certain embodiments, the fluorescence of the sample is decreased by at least 10%, 20%, 30%, 40%, 50%, 60%, 70%, 80%, or 90%.

Provided herein are methods of detecting a target, the method comprising: (i) contacting the target with a fluorogenic sensor provided herein; and (ii.a) measuring or observing the fluorescence of the fluorogenic sensor or (ii) measuring or observing the change in fluorescence lifetime of the fluorogenic sensor. As described herein, the fluorescence of the sample may increase upon binding of the fluorogenic sensor to the target. In certain embodiments, the fluorescence lifetime of the fluorogenic sensor may change upon binding of the fluorogenic sensor to the target. In certain embodiments, this is possible without the need to add additional components (i.e., FRET donor/accepter), an advantage over previous sensors.

Provided herein are methods of detecting an antigen, the method comprising: (i) contacting the antigen with a fluorogenic sensor provided herein; and (ii.a) measuring or observing the fluorescence of the fluorogenic sensor or (ii.b) measuring or observing the change in fluorescence lifetime of the fluorogenic sensor.

In certain embodiments, the fluorescence is increased by at least 10% upon binding to the target (e.g., antigen). In certain embodiments, the fluorescence is increased by at least 10%, 20%, 30%, 40%, 50%, 60%, 70%, 80%, or 90% upon binding to the target (e.g., antigen). In certain embodiments, the fluorescence of the sensor is increased by at least 1-fold, 2-fold, 5-fold, 10-fold, 20-fold, 25-fold, 30-fold, 35-fold, 40-fold, 45-fold, 50-fold, 100-fold, 150-fold, 200-fold, 300-fold, 400-fold, or 500-fold. In certain embodiments, the increase in fluorescence is greater than 500-fold.

In certain embodiments, the fluorescence is decreased by at least 10% upon binding to the target (e.g., antigen). In certain embodiments, the fluorescence is decreased by at least 10%, 20%, 30%, 40%, 50%, 60%, 70%, 80%, or 90% upon binding to the target (e.g., antigen).

Fluorescence can be measured or observed by means known in the art. For example, in certain embodiments, the fluorescence is measured or observed by fluorescence spectroscopy (e.g., using a fluorometer). In certain embodiments, the fluorescence is observed by microscopy. In certain embodiments, the fluorescence is observed visually (e.g., with the naked eye). In certain embodiments, the detection is colorimetric.

Methods provided herein allow for rapid (e.g., instantaneous) detection of targets (e.g., antigens). In certain embodiments, an increase in fluorescence is observed within under 1 second of the contacting step. In certain embodiments, an increase in fluorescence is observed within under 2500, 2000, 1500, 1000, 750, 500, or 250 milliseconds (ms) of the contacting step. In certain embodiments, an increase in fluorescence is observed within under 2500 ms of the contacting step. In certain embodiments, an increase in fluorescence is observed within under 2000 ms of the contacting step. In certain embodiments, an increase in fluorescence is observed within under 1000 ms of the contacting step. In certain embodiments, an increase in fluorescence is observed within under 500 ms of the contacting step. In certain embodiments, an increase in fluorescence is observed within under 250 ms of the contacting step. In certain embodiments, an increase in fluorescence is observed within under 100 ms of the contacting step.

Rapid (e.g., instantaneous) detection of targets (e.g., antigens) can allow for diagnostic methods with little to no significant wait time. This includes rapid (e.g., instantaneous) detection of SARS-CoV-2 viruses, influenza viruses, and other pathogens such as bacteria. The methods also allow for rapid (e.g., instantaneous) detection of targets in other time-sensitive settings, such as during surgery or operation. Therefore, the methods described herein have intraoperation surgical application such as intraoperative specific staining to detect certain biomarkers during surgery.

In-situ detection of targets can allow for instant detection of an analyte across a variety of settings including rapid identification of food spoilage in a warehouse, or instant detection of controlled substances in a law enforcement or military setting.

In certain embodiments, the antigen to be detected is a pathogen. In certain embodiments, the pathogen is a virus. In certain embodiments, the virus is a coronavirus or variant thereof. In certain embodiments, the virus is a SARS-CoV-2 virus or a variant thereof. In certain embodiments, the SARS-CoV-2 variant is the Alpha, Beta, Dela, Gamma, or Omicron variant. In certain embodiments, the SARS-CoV-2 variant is a future variant (i.e., a variant not yet discovered or in existence). In certain embodiments, the SARS-CoV-2 variant is an Omicron variant described herein.

In certain embodiments, the target to be detected is an EGFR protein. In certain embodiments, the target to be detected is cortisol (e.g., cortisol sulfate). In certain embodiments, the target to be detected is ALFA protein (i.e., ALFA-tag).

Also provided herein are kits comprising a fluorogenic sensor provided herein. In certain embodiments, the kit is useful for detecting a pathogen (e.g., virus, e.g., SARS-CoV-2 or a variant thereof) according to a method described herein. Optionally, a kit provided herein will include instructions for use.

### EXAMPLES

### Evolution of New Biosensors

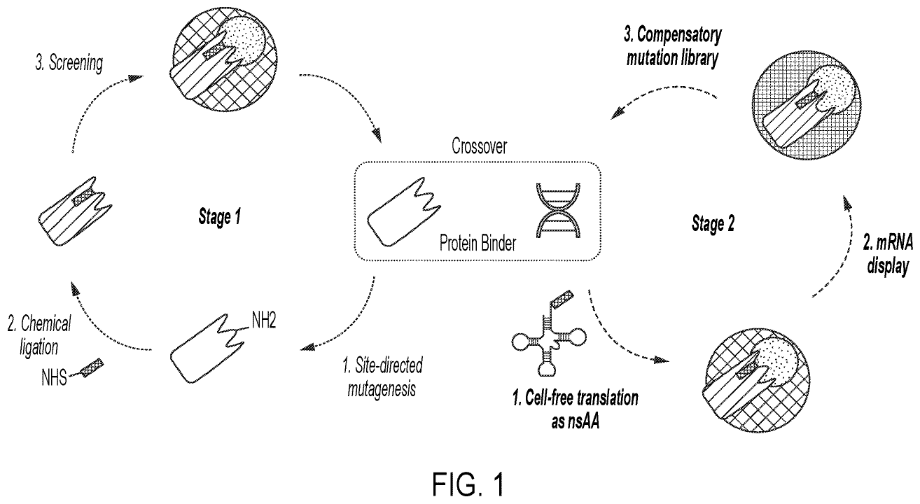

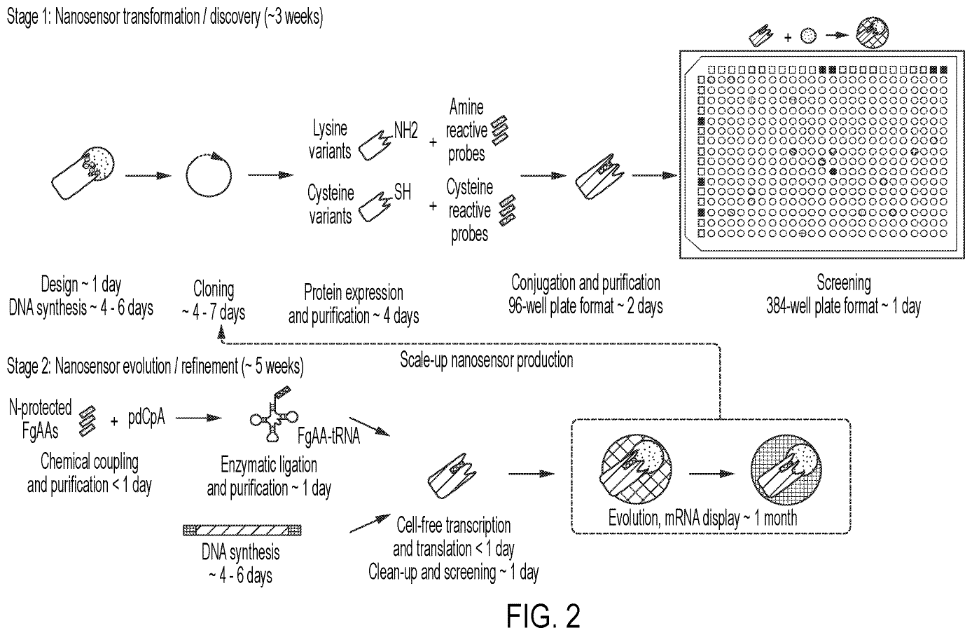

Herein is described a 2-staged, synthetic biology pipeline to rapidly discover, produce, and evolve optical biosensors based around fluorogenic amino acids (FIG. 1 and FIG. 2). The first stage relies on the derivatization of (e.g., <15 kDa) protein binders with cysteine- or lysine-reactive fluorogens to small biosensors, i.e., nanosensors, under scalable and cost-effective manufacturing procedures (>25 mg from 1 L E. coli culture). The multiplexed exploration of hundreds of fluorogen-spacer-residue combinations enables nanosensor discovery. The second (evolution) stage capitalizes on a highly efficient tRNA charging chemistry for cell-free ribosomal translation of nanosensors and their optimization for specific targets via directed evolution. Together, this platform allows rapid molecular design of biosensors with applications in diagnostics, bio-surveillance and molecular imaging.

The main challenge in biosensor discovery is the requirement to empirically determine the optimal fluorogenic probe, linker, and position combinations^{2,6}. The currently limited repertoire of genetically encodable fluorescent and fluorogenic amino acids led to a modular and high-throughput biosensor discovery platform (FIG. 2).

Wuhan-NS (disclosed as “NanoX” in International PCT Patent Application No. PCT/US2022/021878, published as WO 2022/204475 A1, the entire contents of which is incorporated herein by reference) interacted moderately with the Omicron B.1.1.529 strain RBD (RBD_{OB.1}) (K_{D}>9 μM, Table 1). Relevant nanobodies and nanosensors were characterized by Biolayer Interferometry (BLI) to measure their binding affinities (K_{D}). Data shows a similar K_{D} for the unmodified Wuhan VHH72 wild type nanobody and its nanobody variant (VHH72-NoK) in which four framework lysines were mutated to isoelectric arginines (K43R, K65R, K76R, K87R). Wuhan-NS has a 6-times higher K_{D} than the WT nanobody. Wuhan-NS binds RBD_{W} with 30-fold higher affinity than RBD_{OB.1} The affinity of the nanosensors after mRNA/cDNA display directed evolution is similar to or better than the wild type nanobody. Fit parameters: maximal binding parameter (Rₘₐₓ) and goodness of fit (R²).

TABLE 1

| Nanosensor | Target | K_{D}(nM) | Rₘₐₓ | R² |
|---|---|---|---|---|
| VHH72 wt | RBD_{W} | 45 | 1.29 | 0.96 |
| VHH72 NoK wt | RBD_{W} | 75 | 0.45 | 0.99 |
| Wuhan-NS | RBD_{W} | 290 | 1.52 | 0.99 |
| Wuhan-NS | RBD_{OB.1} | 9532 | 2.70 | 0.97 |
| Omicron-NS-1 | RBD_{OB.1} | 37 | 1.14 | 0.83 |
| Omicron-NS-2 | RBD_{OB.1} | 95 | 1.36 | 0.83 |
| Omicron-NS-3 | RBD_{OB.1} | 87 | 1.19 | 0.98 |

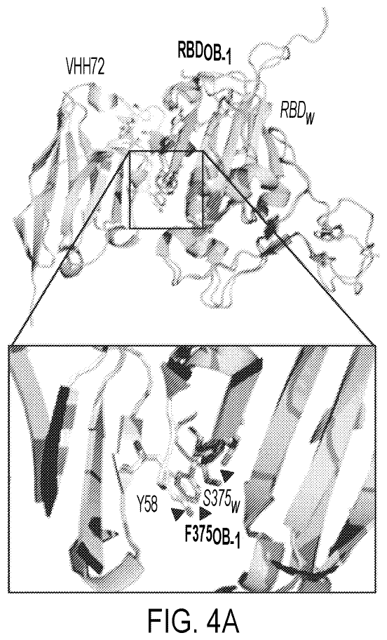

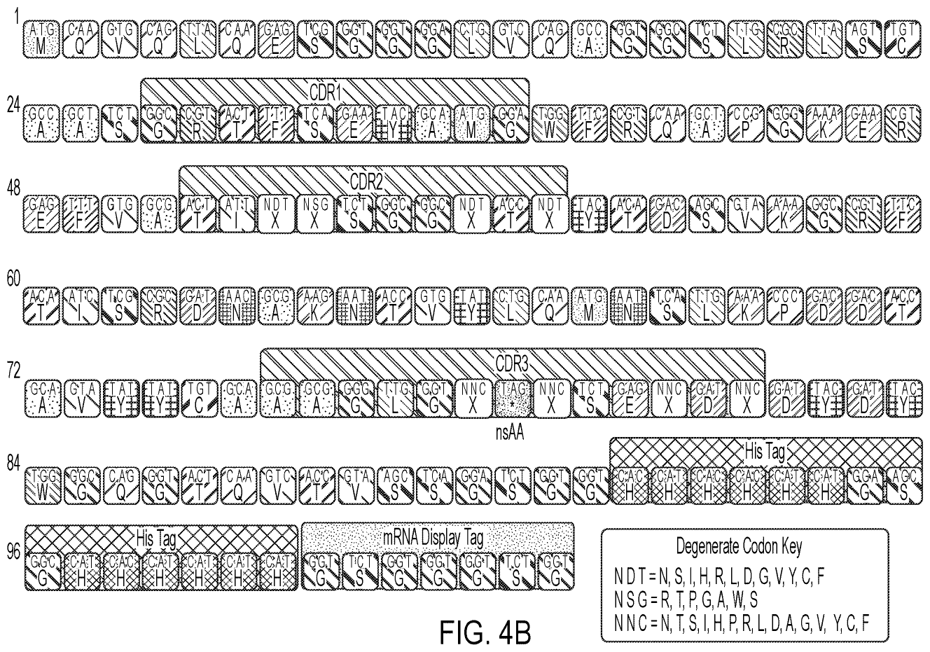

A directed biosensor evolution pipeline can yield improve sensors for emerging variants like RBD_{OB.1} by selecting from sensor libraries sampling compensatory mutations (FIG. 4). A robust biosensor evolutionary pipeline, however, should avoid a two-step probe conjugation chemistry which can bias the selection of unreacted protein variants due to suboptimal probe reactivities. This can only be ensured by one-step, genetic construction of nanosensors by site-specific incorporation of the ‘mature’ fluorogenic residue as one non-standard amino acid (nsAA) unit, e.g., NBDxK for Wuhan-NS.

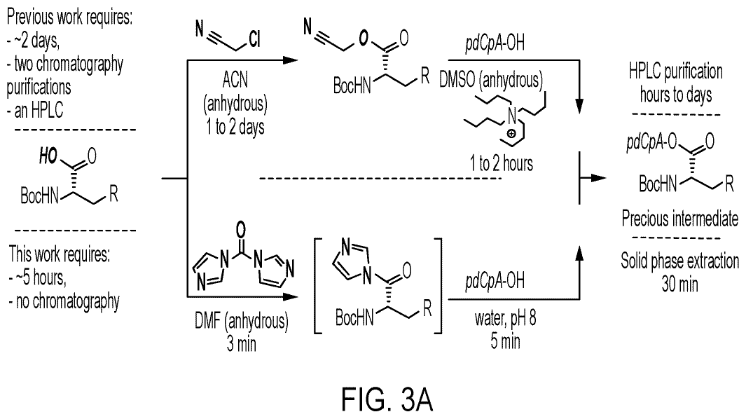

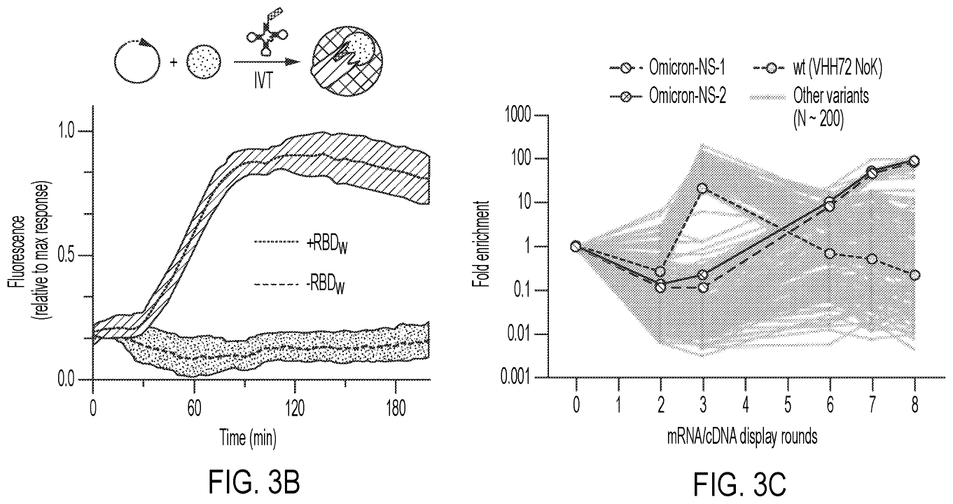

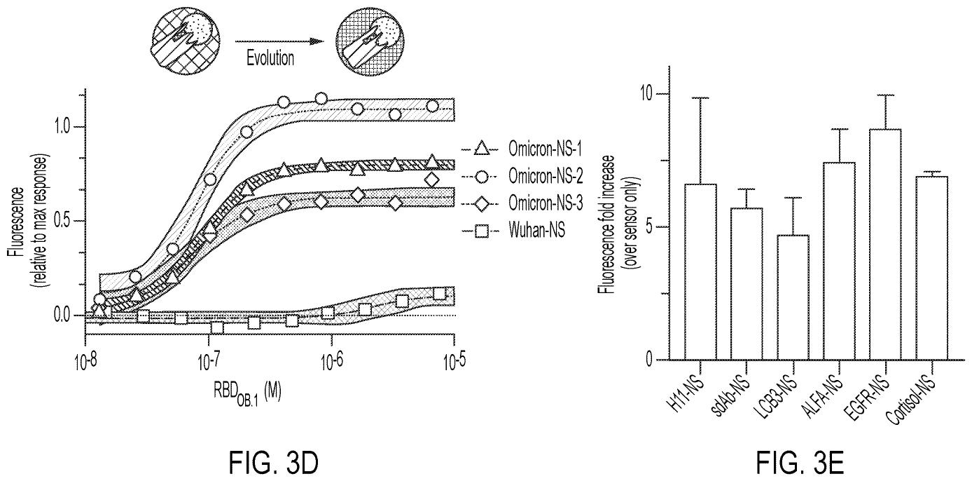

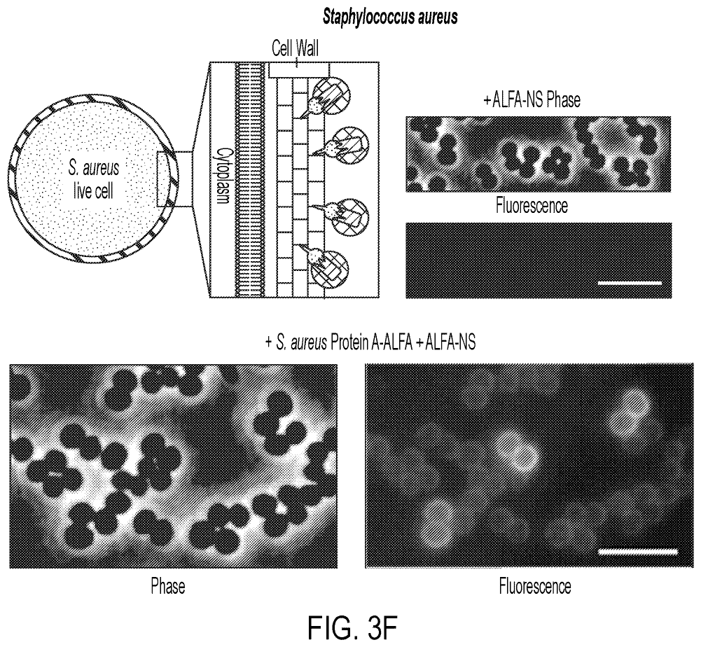

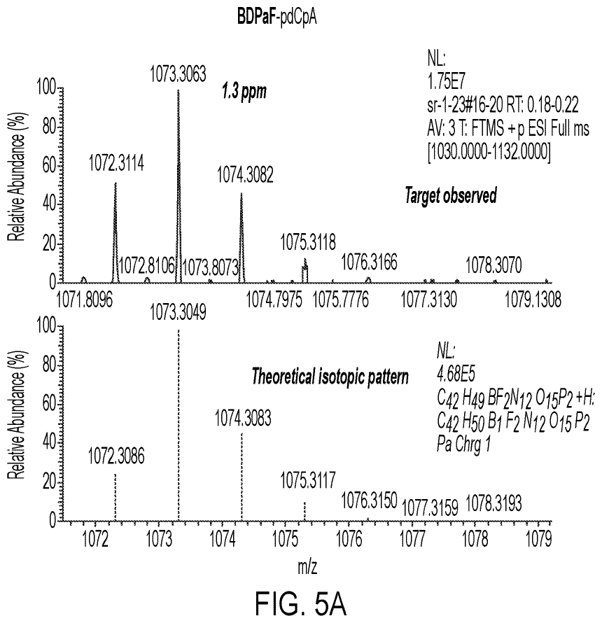

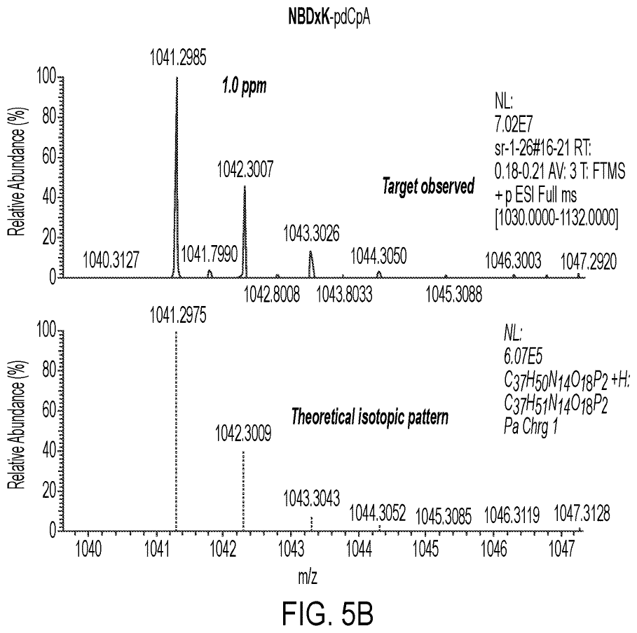

To construct Wuhan-NS ribosomally, the approach is to chemically acylate NBDxK to an amber-decoding orthogonal tRNA_{CUA} via a pdCpA dinucleotide intermediate that could be encoded into a VHH72 V104UAG construct by amber suppression in vitro (FIG. 2). Although various nsAAs have been successfully encoded using similar approaches¹⁷⁻²⁰, these procedures involve multiple purification steps with low recovery yields. As described herein, the chemical synthesis of nsAA-pdCpA conjugates (FIG. 3A) was optimized with a short protocol, involving activation of Boc-protected nsAAs with CDI in dry DMF followed by immediate mixing with pdCpA in water at pH˜8. Reactions had conversion rates up to 70% in under 10 min and did not require HPLC purification. The approach was validated with the brightly fluorescent (therefore easy to detect) amino acid p-(BODIPY-FL)-aminophenylalanine (BDPaF),²⁰ which was then site-specifically incorporated into different positions of proteins of various sizes (nanobody scaffolds, sfGFP, and lysozyme) in vitro by amber suppression (FIGS. 5A, 6, 7). Next, the same protocol was applied to the fluorogenic amino acid (FgAA), NBDxK. It was demonstrated that site-specific ribosomal incorporation of NBDxK (validated by MS and in-gel fluorescence, FIGS. 5B, 8, 9) can produce active Wuhan-NS that can report on RBD_{W} in real-time as the nascent nanosensor is being translated due to the binding-activated fluorescence of NBDxK (FIG. 3B).

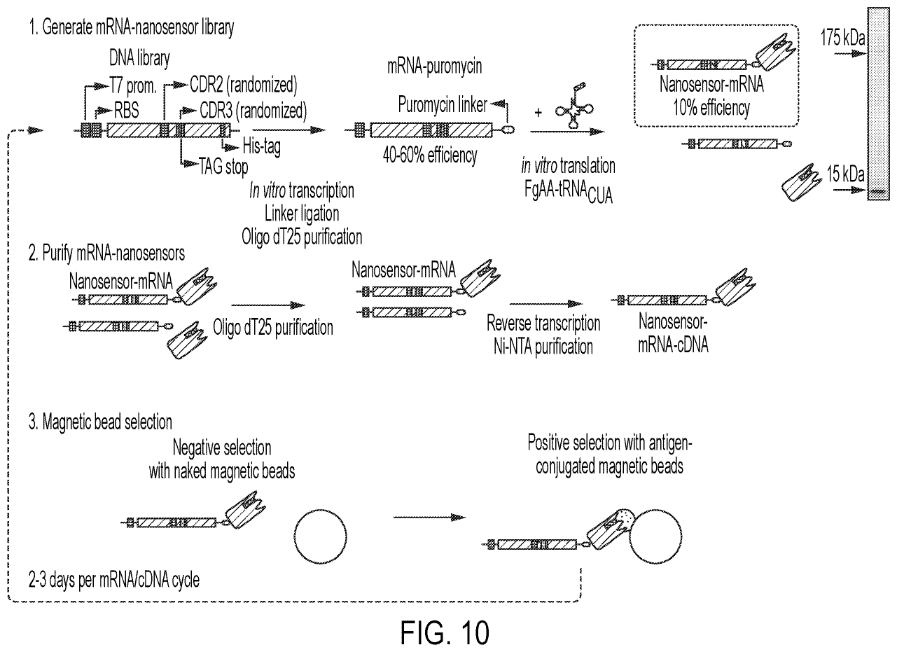

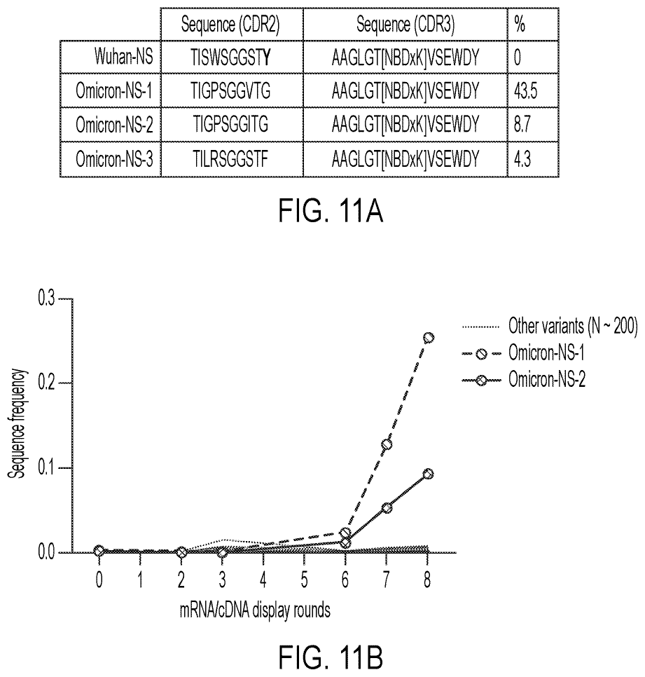

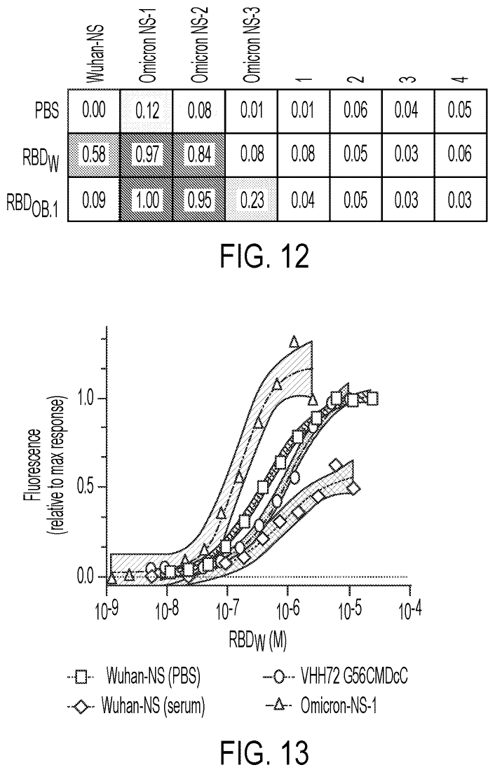

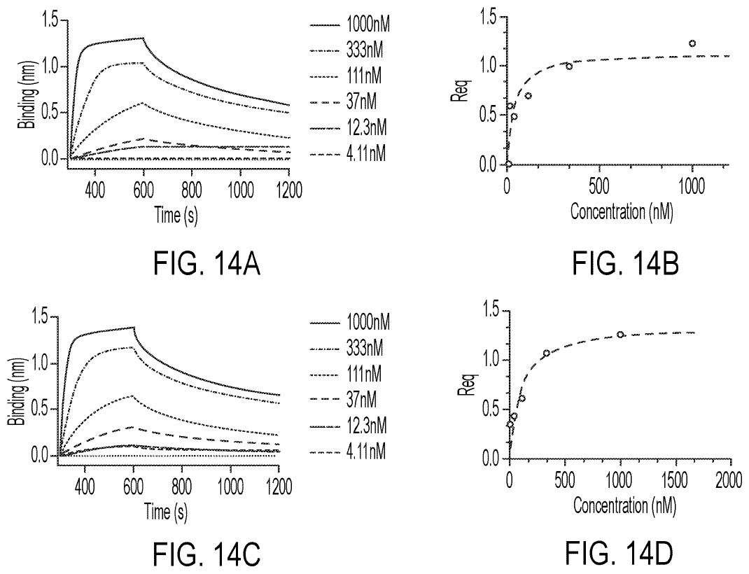

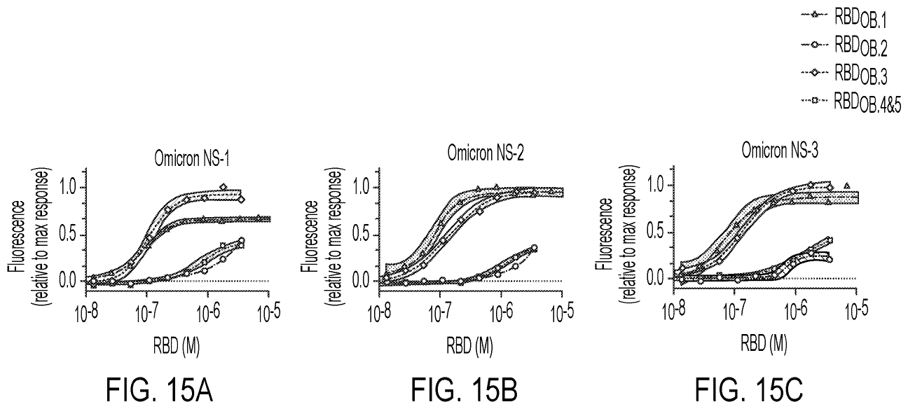

This optimized genetic code expansion chemistry was applied to establish a biosensor evolution pipeline. The strategy relies on mRNA/cDNA display of nsAA-containing nanobodies, which allows up to two rounds of selection per week (FIG. 10). Seven rounds of selection from a ˜10⁸ Wuhan-NS compensatory mutation library (FIG. 4B) against RBD_{OB.1} resulted in convergence to distinct variants (FIG. 11). The rapid screening of 3 enriched variants after one-step ribosomal production and without purification showed that these variants strongly responded to both RBD_{OB.1} and RBD_{W} (FIG. 12). Furthermore, by manufacturing these sensors in larger scale in E. coli (FIG. 2) it was determined that these new nanosensors (Omicron-NS-1-3, corresponding to SEQ ID NOs: 1-3, respectively) had both ˜3 fold improved affinity in RBD_{W} sensing, (EC₅₀˜150 nM, FIG. 13) and a dramatically improved performance in RBD_{OB.1} sensing than Wuhan-NS with ˜250 fold higher affinity (K_{D}˜40 nM) and ˜10-fold increased brightness (FIG. 14, Table 1, FIG. 3D). Omicron-NS-1/3 also selectively responded to RBD_{OB.1} and RBD_{OB.3} but not to other Omicron variants, such as RBD_{OB.2} and RBD_{OB.4.5} (FIG. 15, Table 2).

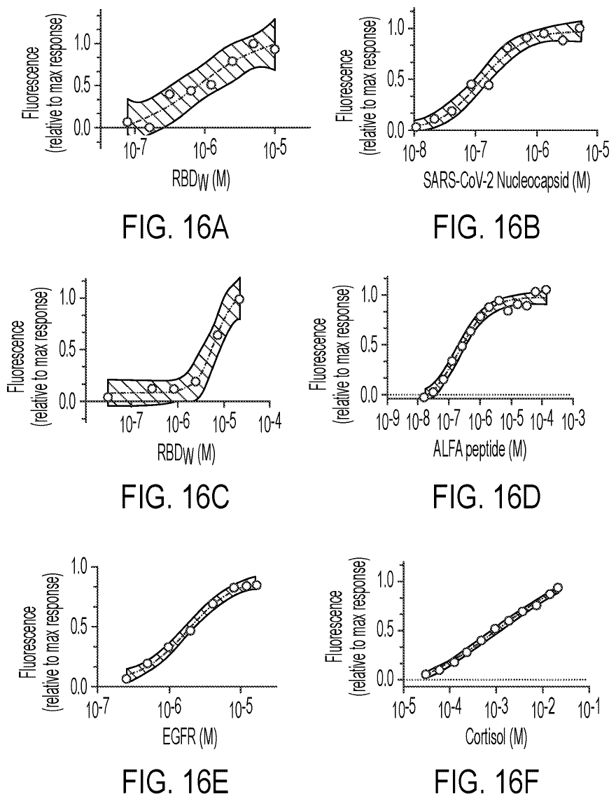

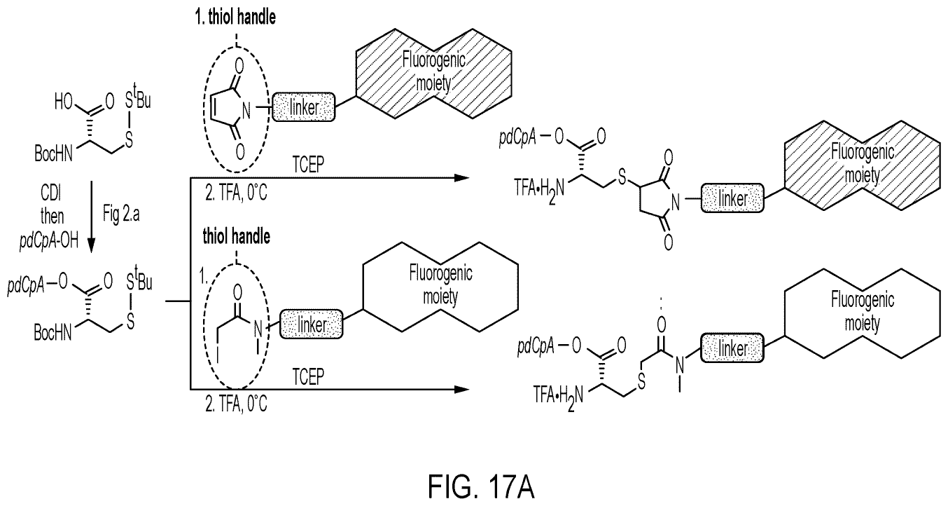

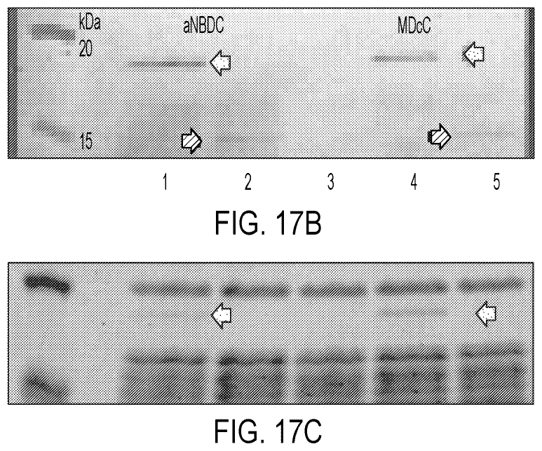

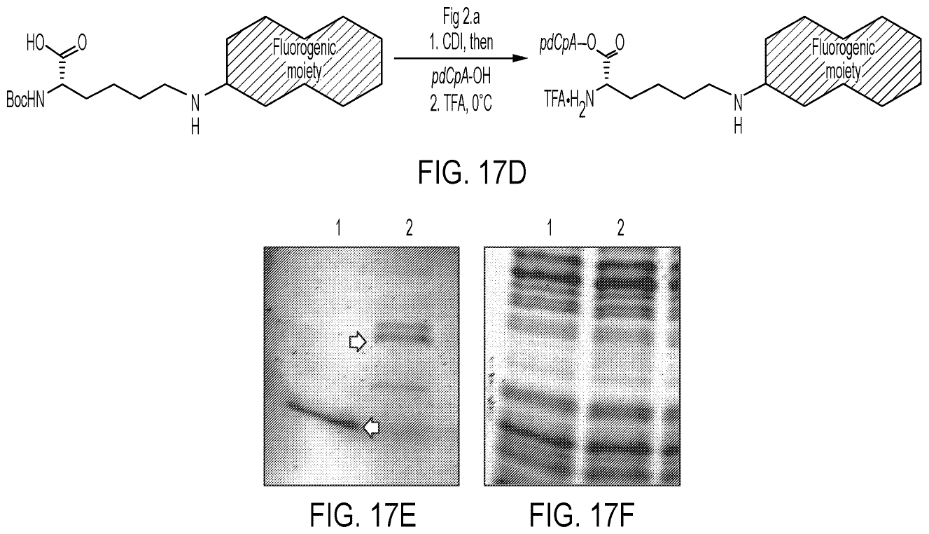

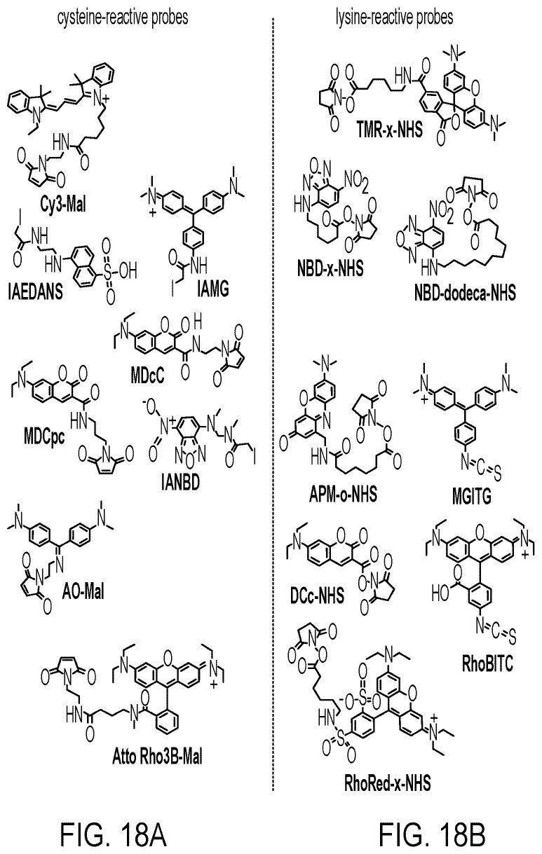

To demonstrate the generalizability of this platform to a wide range of binders and antigens, engineered biosensors based on different scaffolds were prepared: (1) the nanobody H11-H4 against SARS-CoV-2 RBD,²¹ (2) the nanobody sdAb-B6 against the SARS-Cov-2 nucleocapsid protein,²² (3) the miniprotein (<8 kDa) LCB3 against SARS-CoV-2 RBD,²³ (4) the nanobody NbALFA against the genetically encodable ALFA-tag peptide (˜1.5 kDa)²⁴, (5) the nanobody EgA1 against the human epidermal growth factor receptor (EGFR),²⁵ and (6) the nanobody NbCor against a small molecule, cortisol.²⁶ The multiplexed screening of ˜1000 candidates resulted in new nanosensors that can function over a broad range of target concentrations (FIG. 3E, FIG. 16, Table 2). Notably, the ALFA-tag nanosensor can be used as a wash-free, live-cell stain for otherwise difficult-to-track surface proteins, such as the Protein A in the human pathogen Staphylococcus aureus (FIG. 3F). Finally, leveraging the modular nsAA-pdCpA diversification strategy, also validated the genetic encodability of three FgAAs (other than NBDxK) which constitute common ‘mature’ fluorogenic residues in these nanosensors and many other successful biosensors derived from different protein binders^{27,28} (FIG. 17). Taken together, this biosensor discovery and evolution platform is broadly applicable to different scaffolds and FgAAs.

TABLE 2

| Nanosensor | Target | EC50 | | 95% CI | | R² |
|---|---|---|---|---|---|---|
| Wuhan-NS | RBD_{W} | 420 | nM | 345-508 | nM | 0.997 |
| Wuhan-NS | RBD (del 69-70 N439K) | 1.1 | μM | 0.8-1.6 | μM | 0.984 |
| Wuhan-NS | RBD (E484Q L452K) | 1.4 | μM | 0.93-1.6 | μM | 0.983 |
| Wuhan-NS | RBD_{OB.1} | ~2.4 | μM | (Very wide) | | 0.71 |
| Wuhan-NS | RBD (MERS) | N/A | | (Very wide) | | N/A |
| Wuhan-NS | RBD (Y453F) | 0.79 | μM | 0.49-1.96 | μM | 0.989 |
| Wuhan-NS | RBD (CoV-1) | 123 | nM | 80.5-222 | nM | 0.988 |
| Wuhan-NS | RBD_{W} (Serum) | 0.74 | μM | 0.4-13.9 | μM | 0.966 |
| VHH72 G56CMDcC | RBD_{W} | 1.1 | μM | 0.8-1.6 | μM | 0.995 |
| Omicron-NS-1 | RBD_{W} | 154 | nM | 100-243 | nM | 0.974 |
| Omicron-NS-1 | RBD_{OB.1} | 88 | nM | 75.7-102 | nM | 0.997 |
| Omicron-NS-1 | RBD_{OB.2} | ~19 | mM | (Very wide) | | 0.992 |
| Omicron-NS-1 | RBD_{OB.3} | 106.8 | nM | 89.2-128 | nM | 0.993 |
| Omicron-NS-1 | RBD_{OB.4&5} | 786 | nM | 0.56-1.54 | μM | 0.985 |
| Omicron-NS-2 | RBD_{OB.1} | 81.3 | nM | 59.7-104 | nM | 0.987 |
| Omicron-NS-2 | RBD_{OB.2} | ~5 | mM | (Very wide) | | 0.996 |
| Omicron-NS-2 | RBD_{OB.3} | 129 | nM | 105-161 | nM | 0.995 |
| Omicron-NS-2 | RBD_{OB.4&5} | 1.1 | μM | 0.79-2.9 | μM | 0.986 |
| Omicron-NS-3 | RBD_{OB.1} | 71.2 | nM | 21.8-108 | nM | 0.976 |
| Omicron-NS-3 | RBD_{OB.2} | 695 | nM | 478-924 | nM | 0.942 |
| Omicron-NS-3 | RBD_{OB.3} | 133 | nM | 111-160 | nM | 0.995 |
| Omicron-NS-3 | RBD_{OB.4&5} | 1.5 | μM | 0.85-40.8 | μM | 0.99 |
| H11-NS | RBD_{W} | 779 | nM | (Very wide) | | 0.938 |
| sdAb-NS | SARS-CoV-2 NC | 124 | nM | 82-188 | nM | 0.982 |
| LCB3-NS | RBD_{W} | 6.4 | μM | (Very wide) | | 0.994 |
| ALFA-NS | ALFA peptide | 181 | nM | 82.2-304 | nM | 0.934 |
| EGFR-NS | EGFR | 1.74 | μM | 0.85-2.4 | μM | 0.967 |

| | | | | | |
|---|---|---|---|---|---|
| Cortisol-NS | Cortisol | 0.9 | mM | (Very wide) | 0.986 |

As shown in Table 2, relevant nanosensors were characterized by fitting dose-response curves with 4PL Sigmoidal curves to measure EC50 values and best-fit parameters. Data shows binding parameters for Wuhan-NS against several RBD homologs, as well as binding characteristics oof the Omicron-NS 1, Omicron-NS2 and Omicon-NS3 against RBD homologs. Also shown, the binding parameters of several other nanosensors (H11-NS, sdAb-NS, LCB3-NS, ALFA-NS, EGFR-NS and Cortisol-NS) against their respective targets. As expected, Omicron sensors after mRNA display had a lower EC50 when binding RBD_{W} and RBD_{O}, as compared to the parent nanosensor Wuhan-NS; which correlates well with data measured by BLI. 4PL best-fit values: half maximal effective concentration (EC50), 95% confidence interval (95% CI) and goodness of fit (R²).

Described herein is a new strategy for streamlined engineering of targeted optical biosensors. The long-sought ability to evolve optical biosensors is realized by the ribosomal translation of fluorogenic amino acids via an optimized genetic code expansion chemistry and constitutes a notable example of directed evolution with nsAA-containing proteins. The nanosensors hold potential to address many challenges in research such as instant imaging of cell surface antigens for fundamental studies of dynamic cellular processes. This platform can be also used to track rapidly evolving natural proteins or viruses. For example, the versatility of the platform has been demonstrated to swiftly evolve biosensors for new SARS-CoV-2 variants, which may be critical to the successful containment and surveillance of future outbreaks. Put together, the two-stage streamlined workflow of flexible biosensor engineering, manufacturing and evolution represents a timely advance for the development of low-cost, rapid, and selective biosensors with applications in molecular imaging, diagnostics, and biomolecule sensing.

### REFERENCES

1. Adamson, H. & Jeuken, L. J. C. Engineering Protein Switches for Rapid Diagnostic Tests. ACS Sensors 5, 3001-3012 (2020).
2. de Picciotto, S. et al. Design Principles for SuCESsFul Biosensors: Specific Fluorophore/Analyte Binding and Minimization of Fluorophore/Scaffold Interactions. Journal of Molecular Biology 428, 4228-4241 (2016).
3. Dong, J. & Ueda, H. Recent Advances in Quenchbody, a Fluorescent Immunosensor. 21, 1223 (2021).
4. Brient-Litzler, E., Pluckthun, A. & Bedouelle, H. Knowledge-based design of reagentless fluorescent biosensors from a designed ankyrin repeat protein. Protein Engineering, Design and Selection 23, 229-241 (2009).
5. Mills, J. H., Lee, H. S., Liu, C. C., Wang, J. & Schultz, P. G. A Genetically Encoded Direct Sensor of Antibody-Antigen Interactions. ChemBioChem 10, 2162-2164 (2009).
6. De Lorimier, R. M. et al. Construction of a fluorescent biosensor family. 11, 2655-2675 (2002).
7. McMahon, C. et al. Yeast surface display platform for rapid discovery of conformationally selective nanobodies. Nature Structural & Molecular Biology 25, 289-296 (2018).
8. Hoogenboom, H. R. Designing and optimizing library selection strategies for generating high-affinity antibodies. Trends in Biotechnology 15, 62-70 (1997).
9. Kuru, E. et al. Release Factor Inhibiting Antimicrobial Peptides Improve Nonstandard Amino Acid Incorporation in Wild-type Bacterial Cells. ACS Chemical Biology 15, 1852-1861 (2020).
10. Cheng, Z., Kuru, E., Sachdeva, A. & Vendrell, M. Fluorescent amino acids as versatile building blocks for chemical biology. Nature Reviews Chemistry 4, 275-290 (2020).
11. Wrapp, D. et al. Structural Basis for Potent Neutralization of Betacoronaviruses by Single-Domain Camelid Antibodies. Cell 181, 1004-1015.e1015 (2020).
12. Babendure, J. R., Adams, S. R. & Tsien, R. Y. Aptamers switch on fluorescence of triphenylmethane dyes. Journal of the American Chemical Society 125, 14716-14717 (2003).
13. Benson, S. et al. Photoactivatable metabolic warheads enable precise and safe ablation of target cells in vivo. Nat Commun 12, 2369 (2021).
14. Erez, Y., Amdursky, N., Gepshtein, R. & Huppert, D. Temperature and Viscosity Dependence of the Nonradiative Decay Rates of Auramine-O and Thioflavin-T in Glass-Forming Solvents. The Journal of Physical Chemistry A 116, 12056-12064 (2012).
15. Cohen, B. E. et al. A fluorescent probe designed for studying protein conformational change. Proceedings of the National Academy of Sciences of the United States of America 102, 965-970 (2005).
16. Wang, C. et al. A human monoclonal antibody blocking SARS-CoV-2 infection. Nat Commun 11, 2251 (2020).
17. Hecht, S. M., Alford, B. L., Kuroda, Y. & Kitano, S. “Chemical aminoacylation” of tRNA's. Journal of Biological Chemistry 253, 4517-4520 (1978).
18. Robertson, S. A., Ellman, J. A. & Schultz, P. G. A general and efficient route for chemical aminoacylation of transfer RNAs. Journal of the American Chemical Society 113, 2722-2729 (1991).
19. Leisle, L. et al. Cellular encoding of Cy dyes for single-molecule imaging. eLife 5, e19088 (2016).
20. Kajihara, D. et al. FRET analysis of protein conformational change through position-specific incorporation of fluorescent amino acids. Nature Methods 3, 923-929 (2006).
21. Huo, J. et al. Neutralizing nanobodies bind SARS-CoV-2 spike RBD and block interaction with ACE2. Nature structural & molecular biology 27, 846-854 (2020).
22. Ye, Q., Lu, S. & Corbett, K. D. Structural Basis for SARS-CoV-2 Nucleocapsid Protein Recognition by Single-Domain Antibodies. 12 (2021).
23. Cao, L. et al. De novo design of picomolar SARS-CoV-2 miniprotein inhibitors. 370, 426-431 (2020).
24. Götzke, H. et al. The ALFA-tag is a highly versatile tool for nanobody-based bioscience applications. Nature Communications 10, 4403 (2019).
25. Schmitz, Karl R., Bagchi, A., Roovers, Rob C., van Bergen en Henegouwen, Paul M. P. & Ferguson, Kathryn M. Structural Evaluation of EGFR Inhibition Mechanisms for Nanobodies/VHH Domains. Structure 21, 1214-1224 (2013).
26. Ding, L. et al. Structural insights into the mechanism of single domain VHH antibody binding to cortisol. 593, 1248-1256 (2019).
27. Kim, Y. E., Chen, J., Chan, J. R. & Langen, R. Engineering a polarity-sensitive biosensor for time-lapse imaging of apoptotic processes and degeneration. Nature Methods 7, 67-73 (2010).
28. Hirshberg, M. et al. Crystal Structure of Phosphate Binding Protein Labeled with a Coumarin Fluorophore, a Probe for Inorganic Phosphate. Biochemistry 37, 10381-10385 (1998).

### Materials and Methods

Reagents: Cou, MDcC, MDCpc, RhoBITC and DCc-NHS were purchased from Sigma (Cat. No. 792551, 05019, 07153, 283924 and 36801, respectively). Cy3-Mal was from Lumiprobe (Cat. 11080), IAEDANS, IANBD, MGITC and RhoRed-x-NHS were from Thermofisher (Cat. 114, D2004, M689 and R6160, respectively). Atto Rho3B-Mal was from ATTO-Tec (Cat. AD Rho3B-41), NBD-dodeca-NHS was fromo chemodex (Cat N0147). TMR-x-NHS and NBD-x-NHS were from AnaSpec (Cat AS-81127 and AS-81213, respectively). 5-iodoacetemido-malachite green (IAMG) was custom synthesized by TOCRIS. AO-Mal and APM-X-NHS were synthesized as described below. Stock solutions were prepared in anhydrous DMSO avoiding prolonged exposure to the room temperature and stored in −80° C. pdCpA was purchased from Dharmacon. PylT tRNA(-CA)_{CUA}¹ and Mycoplasma capricolum Trp1(-CA)_{CUA}² were ordered from Agilent (Table 5). The RBD antigens were purchased from Genscript: SARS-CoV-2 Spike RBD, Wuhan (Cat. No. Z03491), SARS-CoV-2 Spike protein RBD, E484Q, L452R (Cat. No. CP0007), SARS-CoV-2 Spike protein S1, del 69-70, N439K (Cat. No. Z03524), SARS-CoV-2 Spike protein RBD, K417N, L452R, T478K (Cat. No. Z03689), SARS-CoV-2 Spike protein RBD, E484K, K417N, N501Y (Cat. No. Z03537); and from Acro Biosystems: SARS-CoV-2 Spike RBD, B.1.1.529/Omicron (Cat. No. SPD-C522e), SARS-CoV-2 Spike RBD, BA.2.12.1/Omicron (Cat. No. SPD-C522q), SARS-CoV-2 Spike RBD, BA.3/Omicron (Cat. No. SPD-C522i), SARS-CoV-2 Spike RBD, BA.4 BA.5/Omicron, (Cat. No. SPD-C522r). SARS-CoV-2 Nucleocapsid protein was purchased from Genscript (Cat. No. Z03480). ALFA elution peptide was purchased from NanoTag Biotechnologies (Cat. No. N1520). Human epidermal growth factor receptor (EGFR) was purchased from Genscript (Cat. No. Z03194). Cortisol sulfate was purchased from Sigma (Cat. No. SMB00980).

### Synthesis of Fluorogenic Probes

### 1-(2-((bis(4-(dimethylamino)phenyl)methylene)amino)ethyl)-1H-pyrrole-2,5-dione (AO-Mal)

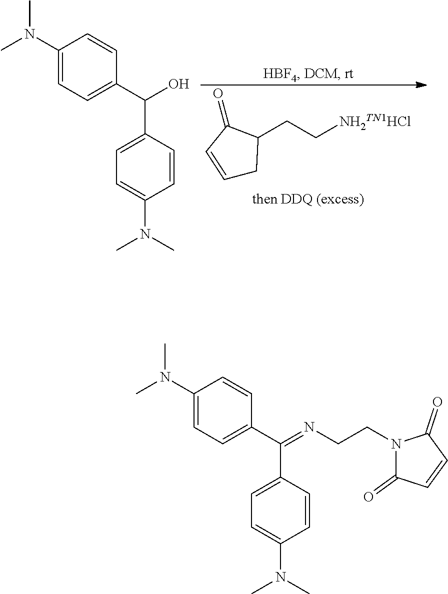

Substrate (40 mg, 0.15 mmol) was dissolved in DCM to which tetrafluoroboric acid (48 wt. % in water) was added drop-by-drop until it became a persistent blue color. Then, the aminoethyl maleimide hydrochloride was added (12 mg, 0.07 mmol). After stirring for 5 minutes, DDQ was added (excess) and the reaction continued for several more minutes. The reaction solution was washed with basic water (NaOH, 0.1 M), then acidic water (HCl, 0.1 M), then washed with brine and finally dried over MgSO₄. The desired product was then purified by CombiFlash (method, mobile phase A: hexane (100%), B EtOAc:EtOH:AcOH (80:20:2%); 0% B isocratic for 10 minutes at 1 mL/min, then from 0% B to 100%). ESI-MS m/z [M+H⁺]⁺ Calc. for [C23H27N4O2]⁺=391.2 matched at 391.2.

### 2,5-dioxopyrrolidin-1-yl 8-(((7-(dimethylamino)-3-oxo-3H-phenoxazin-1-yl)methyl)amino)-8-oxooctanoate (APM-o-NHS)

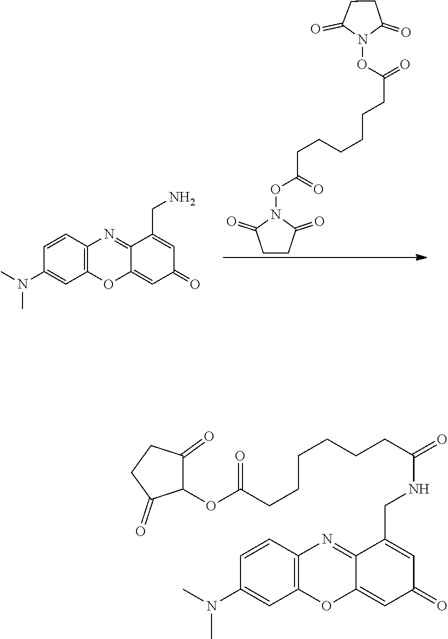 [SMILES: CN(C)c1ccc2nc3c(CN)cc(=O)cc-3oc2c1 ; O=C(CCCCCCC(=O)ON1C(=O)CCC1=O)ON1C(=O)CCC1=O ; CN(C)c1ccc2nc3c(CNC(=O)CCCCCCC(=O)OC4C(=O)CCC4=O)cc(=O)cc-3oc2c1]

Disuccinimidyl suberate (250 mg, MW 368.3, 0.68 mmol) was dissolved in DMF (5 mL) and stirred at room temperature. Powdered L-(aminomethyl)-7-(dimethylamino)-3H-phenoxazin-3-one (APO, 50 mg, MW 269.3, 0.64 mmol) was mixed in over 30 minutes. The reaction was stirred overnight, and the crude reaction was concentrated under reduced pressure. The sample was then dissolved (DCM:MeOH 95:5) and purified by column chromatography (silica, DCM:MeOH 95:5) to give the title compound (60 mg, 68% yield) as a dark purple solid, MS (m/z, ESI): calculated for C₂₇H₃₀N₄O₇⁺ [M+H]⁺ 523.2, found 523.2.

### Synthesis of Fluorogenic Amino Acids (FgAAs)

### (S)-2-((tert-butoxycarbonyl)amino)-3-(7-(diethylamino)-2-oxo-2H-chromene-3-carboxamido)propanoic acid (Boc-DCcaK)

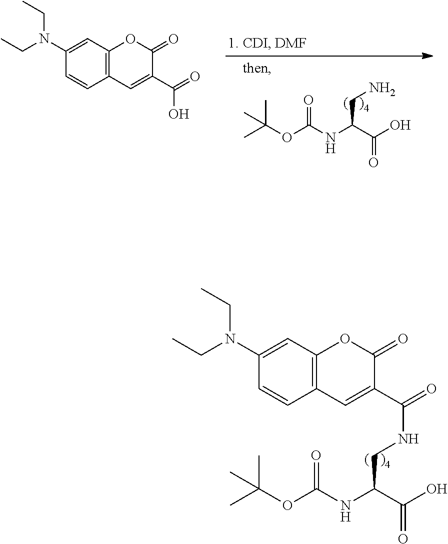

7-(Diethylamino)coumarin-3-carboxylic acid (50 mg, MW 261.3, 0.19 mmol) was dissolved in DMF (1.9 mL) by stirring at room temperature and CDI (37 mg, 0.23 mol, 1.2 eq) was stirred in. After allowing the reaction to continue for 30 minutes, Boc-Lys-OH (51., 0.21 mmol, 1.1 eq) was added in one portion. The reaction continued for several hours, and the crude reaction was extracted, concentrated and purified by CombiFlash [mobile phase A: hexane (100%), B EtOAc:EtOH:AcOH (80:20:2%); 0% B isocratic for 10 minutes at 1 mL/min, then from 0% B to 100% over 100 column volumes at 18 mL/min] to afford the title compound (32 mg, 489.6 g/mol, 0.07 mmol, 34%) as a yellow film. MS (m/z, ESI): calculated for C₂₅H₃₄N₃O₇⁻ [M−H⁺]⁻ 488.2, found 488.3.

### (S)-2-((tert-butoxycarbonyl)amino)-3-(4-(3-(5,5-difluoro-7,9-dimethyl-5H-5l4,6l4-dipyrrolo[1,2-c:2′,1′-f][1,3,2]diazaborinin-3-yl)propanamido)phenyl)propanoic acid (Boc-BDPaF)

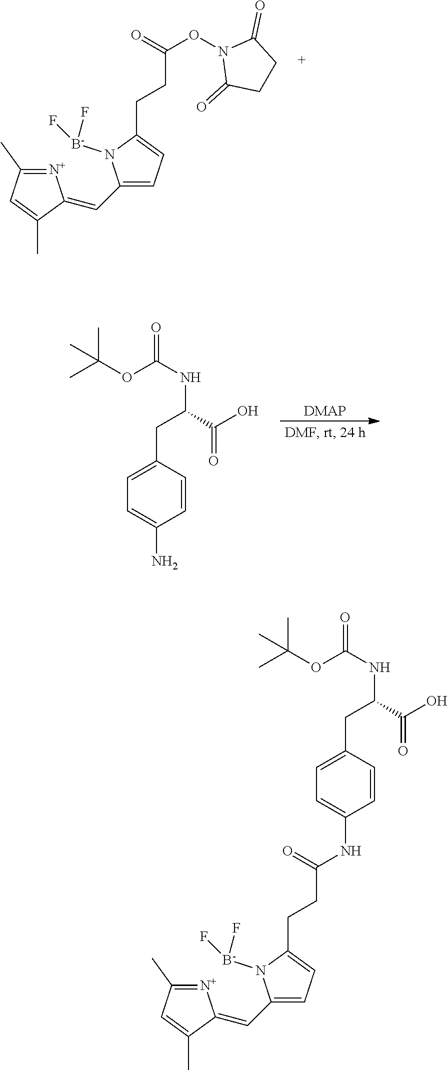

To a stirring solution of Bodipy-FL-NHS (100 mg, 0.26 mmol, 1 equiv), which was dissolved at room temperature in dry DMF (0.5 mL) in a 2-dram vial, Boc-APA (81 mg, 0.29 mmol, 1.1 equiv) was added. DMAP (35 mg, 0.29 mmol, 1.1 equiv) was then added and the resulting reaction mixture was stirred at room temperature for 24 h. The reaction was concentrated under reduced pressure and the crude residue was purified by CombiFlash [mobile phase A: hexane (100%), B EtOAc:EtOH:AcOH (80:20:2%); 0% B isocratic for 10 minutes at 1 mL/min, then from 0% B to 100% over 100 column volumes at 18 mL/min] to afford Bodipy-FL-Boc-APA (93 mg, 65% yield) as orange solid. HRMS-ESI (m/z): Calc. for C₂₈H₃₂BF₂N₄O₅ [M−H⁺]⁻ 553.2439, found 553.2450.

### N2-(tert-butoxycarbonyl)-N6-(6-((7-nitrobenzo[c][1,2,5]oxadiazol-4-yl)amino)hexanoyl)-L-lysine (NBDxK)

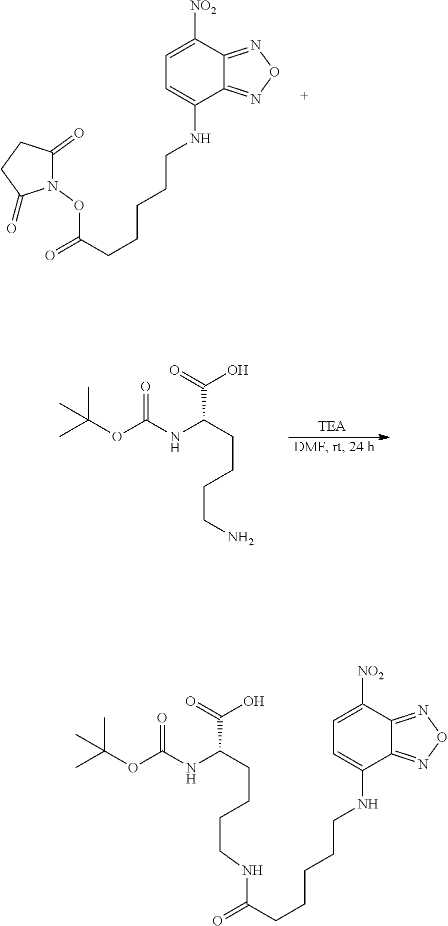 [SMILES: O=C(CCCCCNc1ccc([N+](=O)[O-])c2nonc12)ON1C(=O)CCC1=O ; CC(C)(C)OC(=O)N[C@@H](CCCCN)C(=O)O ; CC(C)(C)OC(=O)N[C@@H](CCCCNC(=O)CCCCCNc1ccc([N+](=O)[O-])c2nonc12)C(=O)O]

To a solution of NBD-x-NHS (117 mg, 391.3 g/mol, 0.30 mmol) in DMF (˜1 mL), Boc-Lys-OH (150 mg, 246.3 g/mol, 0.61 mmol) and TEA (0.2 mL) were added. The reaction was stirred overnight, and the product was isolated by acid/base extraction. The pure product was purified on the CombiFlash (method, mobile phase A: hexane (100%), B EtOAc:EtOH:AcOH (80:20:2%); 0% B isocratic for 10 minutes at 1 mL/min, then from 0% B to 100% over 100 column volumes at 18 mL/min) to afford the desired pure product (26.1 mg) as an orange oily film. HRMS-ESI (m/z): Calc. for C₂₃H₃₃N₆O₈ [M−H⁺]⁻ 521.2365, found 521.2367.

General Procedure for Acylation of pdCpA

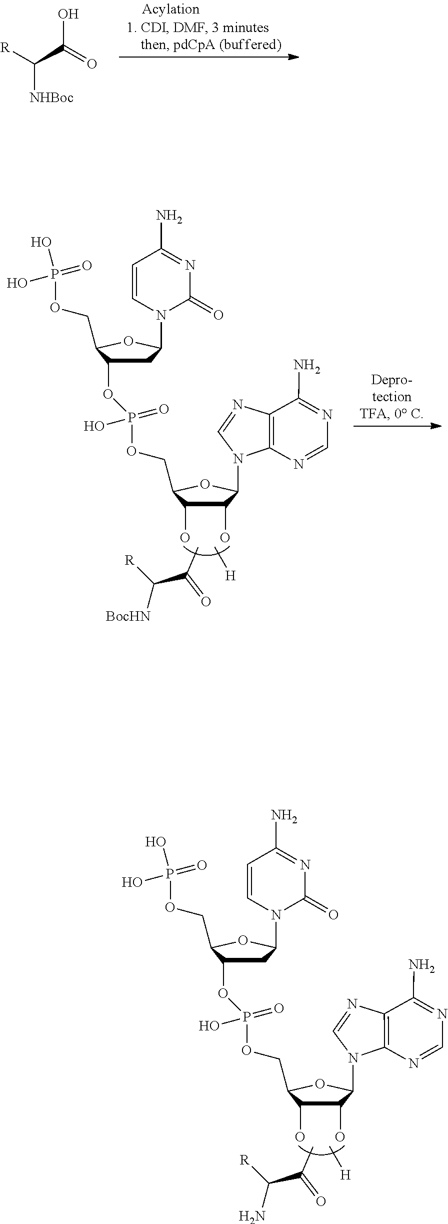

Acylation with Boc-nsAA-OH: A solution of CDI (7 mg, 0.0431 mmol) in dry DMF (45 μL) was added to the Boc-nsAA (12 mg, molar excess) in a 1.5 mL microcentrifuge tube. The resulting reaction mixture was mixed well for 3 minutes and added to an aqueous pdCpA solution (2.5 mg, 3.6 μmol in 55 μL water, pH˜8.3). The resulting heterogeneous, gummy reaction was mixed well with a wide-tipped pipette until a clear solution was observed. The crude reaction mixture was quickly separated into two 2 mL microcentrifuge tubes and diluted with THF. The product was centrifuged and the pellet was quickly washed with fresh THF and dissolved in DI water (˜50 μL). The desired product was then purified by solid-phase extraction via Sep-Pak® C18 cartridges (mobile phase A [˜25 mL, water:HFIP:TEA (1000:42:2)] then mobile phase B [˜5 mL, MeOH:water:HFIP:TEA (500:500:42:2)], flowrate<1 mL/min). Mobile phase B containing product was then diluted in ˜5 mL DI water and lyophilized to provide the Boc-nsAA-OpdCpA. The compound can be stored at −20° C. for weeks. Solid-phase extraction cartridge was performed with.

Boc-Cys(S-^{t}Bu)-OpdCpA that allowed the modular cysteine diversification strategy (FIG. 17) was accessed following the same procedure where Boc-Cys(S-^{t}Bu) was the amino acid partner charged. The lyophilized product was dissolved in 10 mM sodium phosphate buffer (pH=7.5) to which 10 equivalent TCEP was added and the pH was adjusted to ˜7. After incubation at room temperature for 30 minutes, 10 equivalents of thiol reactive maleimide (MDCc) or iodoacetamide (IANBD) probes were added and the reaction progress was followed by LCMS. The desired product was isolated with HPLC and lyophilized to provide the desired product (Boc-aNBDC-pdCpA C₃₉H₅₁N₁₄O₂₀P₂S⁻ [M−H⁺]⁺ calc. 1129.2, found 1129.3; Boc-MDCcC-pdCpA C₄₇H₅₉N₁₂O₂₁P₂S⁻ calc. 1221.3 [M−H⁺]⁺ found 1221.3). Deprotection was accomplished following the procedure below.

Deprotection of Boc-nsAA-OpdCpA: The vial containing Boc-nsAA-OpdCpA was placed on ice and the substrate was transferred to a tared 1.5 mL microcentrifuge tube with TFA cooled to 0° C. The Deprotection was allowed to continue on ice (generally 2 hours). The reaction was then concentrated under a gentle flow of argon until about 5 μL of TFA was left. Diethyl ether was then added to precipitate the free acid and the precipitate was pelleted by centrifuge and the acidic ether was decanted. The pellet stored cold (<−20° C.) or analyzed by HPLC (5-90% acetonitrile over 25 min; mobile phase A [water:HFIP:TEA (1000:42:2),] then mobile phase B [MeOH:water:HFIP:TEA (500:500:42:2)], flowrate<0.4 mL/min) and followed at 260 nm and at λ_{Abs} of the FgAA) on negative mode MS method: neg mode, window=10 Da below mass of Boc-nsAA to 653+2*pg-AA+10. Trace analysis often identified the following peaks: pdCpA, nsAA-OpdCpA (may be several peaks from regioisomers), pdCpA+2 amino acids and the Boc amino acid (Boc-nsAA, as free acid).

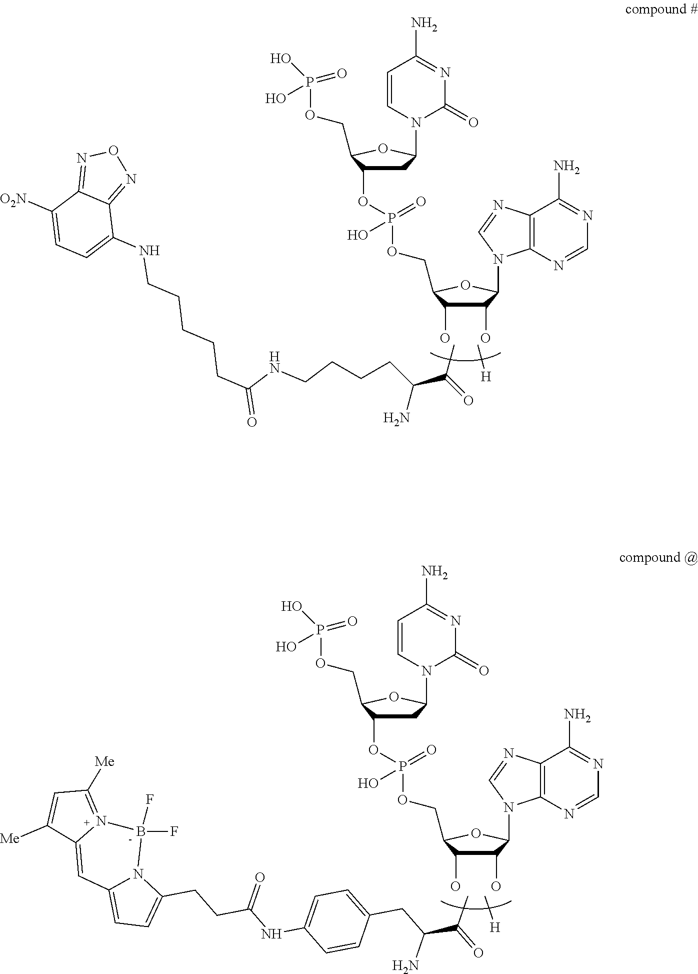

Lys(NBDx)O-pdCpA (Compound #): HRMS-ESI (m/z): Calc. for C₃₇H₅₁N₁₄O₁₈P₂ [M+H]⁺ 1041.2975, found 1041.2985.

4-Amino-Phe(Bodipy)O-pdCpA (Compound @): HRMS-ESI (m/z): Calc. for C₄₂H₅₀BF₂N₁₂O₁₅P₂ [M+H]⁺ 1073.3049, found 1073.3063.

Alternative General Procedure for the Preparation of Non-Standard Amino Acid Charged pdCpA (nsAA-OpdCpA, Compounds A-D)

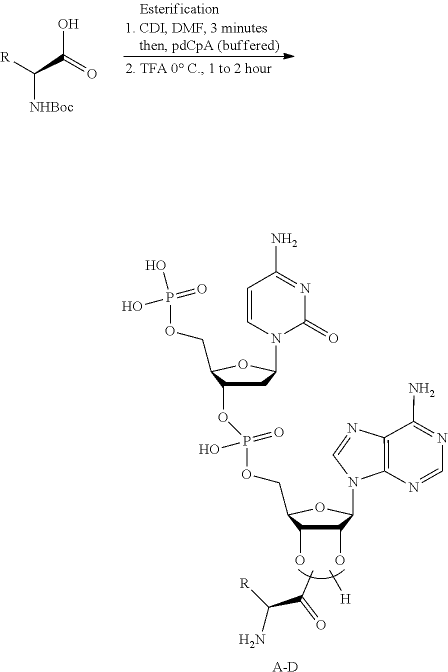

Esterification of Boc-nsAA-OH: A solution of CDI (7 mg, 0.0431 mmol) in dry DMF (45 uL) was added to the solution of Boc-nsAA (12 mg, molar excess) in DMF (45 uL) in an Eppendorf tube. The resulting reaction mixture was mixed well for 3 minutes to activate the Boc-nsAA (generation of bubbles). This solution was then added to a solution of pdCpA (2.5 mg, 3.9 umol) in NaOH (0.05 M, 55 uL, the pH was adjusted to 8.3 with additional NaOH). The resulting heterogeneous, gummy reaction was mixed well with a wide-tipped pipette until a clear solution was observed. The crude reaction mixture was quickly separated into four 2 mL Eppendorf vails and diluted with THF. The product was pelleted on a centrifuge and the THF was decanted. The pellet was then quickly washed with fresh THF and subsequently was dissolved DI water (circa 50 uL). The desired product was then purified by solid-phase extraction (mobile phase A [25 mL (circa), water:HFIP:TEA (1000:42:2)] then mobile phase B [5 mL (circa), MeOH:water:HFIP:TEA (500:500:42:2)], flowrate<1 mL/min). Mobile phase B containing product was then diluted (DI water, 5 mL) and lyophilized to provide the Boc-nsAA-OpdCpA. The compound can be stored at −20° C. for weeks.

Deprotection of Boc-nsAA-OpdCpA: The vial containing the substrate (Boc-nsAA-OpdCpA) was placed on ice and the substrate was transferred to a tared Eppendorf tube with TFA cooled to 0° C. The deprotection was allowed to continue on ice (˜2 hours). The reaction was then concentrated under a gentle flow of argon until about 5 uL of TFA was left. Diethyl ether was then added to precipitate the free acid and the precipitate was pelleted by centrifuge and the acidic ether was decanted (if no precipitate formed, the drying process was repeated). The pellet was then dissolved in DMSO (10 uL) [it is recommended that a little of this product is saved for HPLC analysis]. The product in the DMSO solution was then precipitated (diethyl ether), pelleted and stored cold (−20° C. recommended). HPLC: phase A [water:HFIP:TEA (1000:42:2),] then mobile phase B [MeOH:water:HFIP:TEA (500:500:42:2)], flowrate=0.4 mL/min) and followed at 260 nm and followed on negative mode MS. Methods: 5-90 over 15 min for compound A; 15-90 over 15 min for compound B; 10-90 over 15 min for compound C. Mobile phase A [water:formic acid (1000:1),] then mobile phase B [acetonitrile:formic acid (1000:1)], flowrate=1.0 mL/min) and followed at 254 nm and followed on positive mode MS. Method: 10-90 over 10 min for compound D.

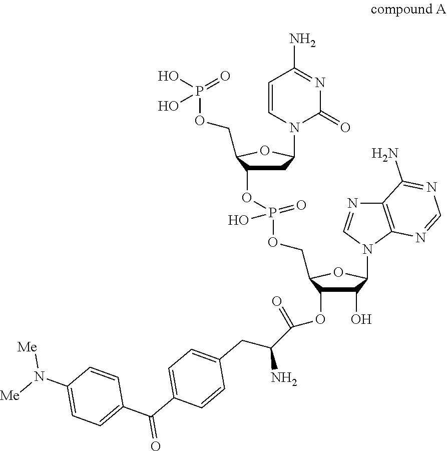

Compound A: This compound was prepared according to the general procedure. LCMS: n/z: [M−H]⁻ Calcd for C₃₇H₄₃N₁₀O₁₅P₂⁻ 929.2; Found 929.3.

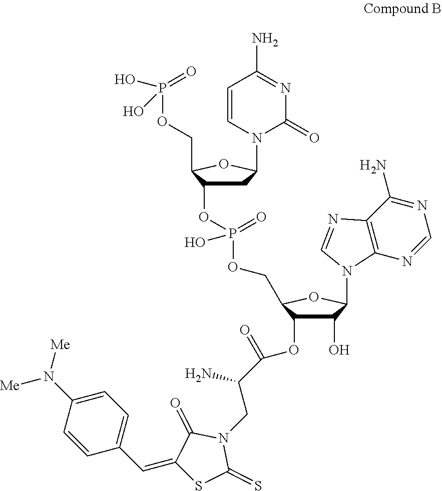

Compound B: This compound was prepared according to the general procedure. LCMS: n/z: [M−H]⁻ Calcd for C₃₄H₄₀N₁₁O₁₅P₂S₂⁻ 968.2; Found 968.2.

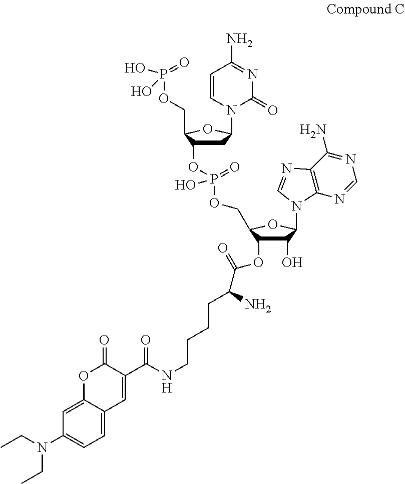

Compound C: This compound was prepared according to the general procedure. LCMS: m/z: [M−H]⁻ Calcd for C₃₉H₅₀N₁₁O₁₇P₂⁻ 1006.3; Found 1006.3.

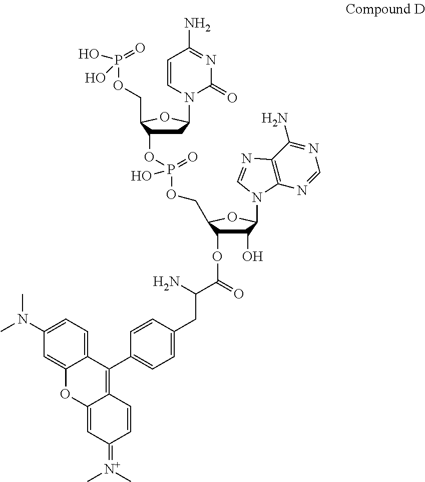

Compound D: This compound was prepared according to the general procedure. LCMS: m/z: [M+H] ²⁺/2 Calcd for C₄₅H₅₃N₁₁O₁₅P₂²⁺ 524.6; Found 524.8 (mixture of diastereomers).

### Cloning of Plasmids Expressing Protein-Binders

For PCR, site-directed mutagenesis, and isothermal assembly procedures Q5® High-Fidelity 2X Master Mix, Q5® Site-Directed Mutagenesis Kit, and Gibson Assembly® Master Mix from New England Biolabs, (NEB Ipswich, MA) were used, respectively, and primers were designed following the manufacturer's instructions. Routinely, new plasmids were constructed assembling linearized backbones of existing plasmids that are optimized for T7 RNA Polymerase dependent expression, i.e. pET-28a (+), pET-28_TEV, or pPURExpress (Table 3), with eBlocks (IDT), and cloning into NEB® 5-alpha Competent E. coli. Nanosensor constructs typically contained N-terminal His tag followed by a Thrombin or TEV cleavage tag, the nanobody sequence and the mRNA display tag. Plasmids expressing EgA1, NbCor, and NbALFA variants were synthesized as Clonal Genes (Twist). All these constructs were verified by Sanger sequencing (Azenta/Genewiz) or complete plasmid sequencing (MGH DNA Core). Constructs for in vitro transcription/translation experiments were cloned into linearized backbone of pPURExpress control plasmid lacking an ORF and sequence verified as described above (Table 3).

### Expression and Purification of Protein-Binder Variants

VHH72 W108Cou were expressed essentially as previously described³ except using a pET-28a plasmid expressing an VHH72 W108UAG ORF with an in-frame amber (UAG) stop codon that was suppressed with tRNA^{Tyr}_{CUA} acylated with Cou by CouRS both expressed from pDule-MjCouRS and was purified as described below (Table 3).⁴

Plasmids expressing protein-binders were routinely transformed into SHuffle® T7 Competent E. coli cells (NEB). Up to 28 different overnight cultures (TB medium, Difco supplemented with 50 μg/mL kanamycin) were used to inoculate 100 mL of TB medium (1:100) supplemented with 50 μg/mL kanamycin at 30° C. and grown to OD600=0.4-0.5 with shaking. Cells were cooled to 16° C. before for overnight induction with 0.75 mM IPTG with shaking. Cells (50 mL×2) were harvested by centrifugation (30 min, 5000 g, RT) and pellets were either stored in −20° C. or resuspended in 4 mL BugBuster® Master Mix (EMD Millipore) and rocked at room temperature for 45 minutes. The lysate was centrifuged (15 min, 5000 g, 4° C.) and the supernatant was added to 0.5 mL HisPur™ Cobalt Resin (Thermo Fisher) that is equilibrated and resuspended in 4 mL Equilibration Buffer (20 mM Tis-HCl pH 8.3, 0.5 M NaCl, 5 mM imidazole) in 15 mL conical tubes. After binding by rotation (35 min, 4° C.), the resin was pelleted (2 min, 700 g, 4° C.) and was washed twice with 1 mL wash Buffer (20 mM Tis-HCl pH 8.3, 0.5 M NaCl, 20 mM imidazole). The protein was eluted by 3×0.5 mL Elution Buffer (20 mM Tis-HCl pH 8.3, 0.5 M NaCl, 200 mM imidazole) and buffer exchanged into 1× Phosphate Buffered Saline (PBS, 137 mM NaCl, 2.7 mM KCl, 10 mM Na₂HPO₄, 1.8 mM KH₂PO₄)+20% glycerol using Zeba™ Spin Desalting Columns, 5 mL, 7K MWCO (Thermo Fisher) and following the manufacturer's instructions. The protein yields and purity were assessed by running 2 μL samples in Novex™ WedgeWell™ Tris-Glycine Protein Gels (10-20%,15-well, Invitrogen) following the manufacturer's instructions. This protocol allowed purification of up to 14 protein binder variants at once typically without the need for further concentration.

### Multiplexed Modification and Screening of Protein Binder Variants and Fluorescence Dose Response Curves

Lysine variants of protein-binders were diluted to 50 μM in 1×PBS supplemented with 50 mM Sodium Borate (pH 8.5) and 45 μL aliquots were added into 96-well PCR plates that contain 5 μL stock solutions of amine-reactive probes (typically 2.5 mM in DMSO). The plate was sealed and the conjugation reaction was incubated for 2 h (dark, 25° C.). Cysteine variants of protein-binders were diluted to 50 μM in PBS supplemented with 500 μM Tris(2-carboxyethyl)phosphine (TCEP), sealed and incubated for 2 h at 25° C. 45 μL aliquots were added into 96-well PCR plates that contain 5 μL stock solutions of thiol-reactive probes (typically 2.5 mM in DMSO). The plate was sealed, and the conjugation reaction was incubated overnight (dark, 4° C.). Labeled proteins were separated from the unreactive probes by size-exclusion chromatography using Thermo Scientific Zeba Spin Desalting 96-well filter plates (7K MWCO), following the manufacturer's instructions. The degree of labeling was assessed by measuring the ratio of fluorophore to protein from absorbance spectra of the purified conjugate and varied typically between ˜0.5-1.5 for amine-reactive probes and ˜0.1-0.8 for thiol-reactive probes. 2.5-10 μL of the resulting conjugates were transferred into low volume 384-well black flat clear bottom plates (Corning) and equal volumes of antigens (at saturating concentrations of typically ˜1 mg/mL or >10 μM) or buffer only (1×PBS) were added. Using Biotek Synergy H1 plate reader, fluorescence measurements were taken either directly in these plates (reading from the bottom) or transferred into Take3 Micro-volume plates (Biotek) at probe-specific optimal excitation/emission wavelengths (Table 4).

Fluorescence readings in the Take3 plates were more sensitive than readings in 384-well plates and thus the maximum fluorescence fold increase values were calculated in Take3 plates. Sensor performance benefited from optimization of labeling during the scaled-up sensor production. For example, optimized Wuhan-NS, that gives the up to 100-fold fluorescence change, relied on modification with NBD-x-NHS ester at 150 μM dye and 10% DMSO followed by thrombin or TEV cleave of the N-terminus due to non-specific labeling of the N-terminal free amines. Dose response curves, e.g., FIG. 13, were determined in black 384-well plates by mixing 10 μL of sensor (at final˜2 μM) with equal volumes of serial dilutions of their corresponding antigens in indicated buffers, e.g. 1×PBS or human serum. The graphs were corrected for the background fluorescence by subtracting the nanosensor signal from the nanosensor plus the antigen signal. Signal values were normalized to peak fluorescence magnitude within an experiment and the graphs were plotted indicating the standard deviation between repeats in shade.

### Biolayer Interferometry

Biolayer interferometry was performed on an Octet Red 384 instrument (ForteBio) at 30° C. with shaking at 1000 rpm. Signals were collected at the default frequency of 5.0 Hz. First, the Streptavidin (SA) biosensors (ForteBio) were preincubated in PBST [1×PBS containing 0.05% Tween 20 (Sigma-Aldrich)], the assay buffer used throughout the whole procedure, for 15 min. Biotinylated RBD—or another specific target for each nanobody—was loaded on the tips at 50 μg/mL. Then, dilutions of the nanosensors and control PBST were associated for 300 s; and dissociated for 600 s. BLI Steady-State analysis was performed in the ForteBio Data Analysis HT Software.

### Immunofluorescence and Image Acquisition

SARS-CoV-2 envelope protein was expressed from a previously described plasmid⁵ that is further modified to exclude 21 amino acids believed to be a cryptic ER-retention signal since the original wild-type envelope protein traffics to the ER-GIC. This resulted in trafficking of Spike protein to the cell membrane which was used to package SARS-CoV-2 pseudotyped lentivirus. 293Ts cells were then transfected with this plasmid vector in the following manner: Chamber slides were coated by applying a solution of 300 μL of 0.5 μg/mL poly-d-lysine in 1×PBS and incubated at 37° C. for 1.5 hours. Chamber slides were then washed once with sterile de-ionized water before cells were seeded into them. Approximately 2.5×10⁴ HEK 293Ts were seeded in a volume of 250 μL complete media (DMEM, 10% FBS, 1% Pen/Strp). After overnight growth at 37° C., 5% CO₂, each well was transfected using PEI. A total of 200 ng SARS-CoV-2 plasmid (or the empty plasmid control) was combined with 600 ng PEI in a final volume of 50 μL DMEM (no FBS, no Pen/Strep). DNA-PEI solution was incubated for 10 minutes at room temperature before combining with 300 μL complete media for a total volume of 350 μL. This volume was then swapped with the existing cell culture supernatant in the chamber slides to transfect the cells. The transfected cells were then returned to the incubator for 48 hours followed by fixation and permeabilization. Supernatants were removed and 200 μL 4% paraformaldehyde diluted in 1×PBS was added to each well and incubated at room temperature for 5 minutes. Each well was carefully washed twice with 1×PBS before 200 μL of 0.1% Triton X-100 in 1×PBS was added to each well and incubated for 10 minutes at room temperature. Each well was then washed 3 times with 200 μL 1×PBS and cells were stored in the same buffer at 4 C for up to 1 week before staining and imaging experiments. At the day of imaging, the media was exchanged into 1×PBS+Wuhan-NS (2.5 μM) and directly imaged. Phase and fluorescence images were acquired using a Nikon Ti2 Eclipse inverted microscope equipped with a Plan Apo Lambda 20X (0.75 NA, DIC N2) oil objective and Andor Zyla sCMOS camera. NIS-Element AR software was used for image acquisition. Image processing was performed in FIJI. Images were scaled, cropped and rotated without interpolation. Linear adjustment was performed to optimize contrast and brightness of the images. Figure construction was performed in Adobe Illustrator.

For data leading to FIG. 3F, S. aureus RN4220 cells were grown in tryptic soy broth (Becton-Dickinson Bacto-TSB, 30 g/L) at 37° C. with aeration, supplemented with 10 μg/mL erythromycin to maintain the plasmid pTB107 when necessary. pTB107 (Table 3) was designed with SnapGene and generated by GenScript using site directed mutagenesis with pLOW as the template, and PCR-verified. Cells were transformed with either empty pLOW vector or pTB107 (pLOW_ALFA-spa-LPXTG), containing an in-frame ALFA tag between the native Staphylococcus protein A (SpA) signal sequence and coding sequence. Experiments were conducted from single colonies grown on tryptic soy agar (TSB with 1.5% Difco bacto-agar) plates. Cells were grown in TSB+10 μg/mL erythromycin overnight into stationary phase, subcultured 1:2000 in fresh TSB, and induced with 50 ng/mL IPTG 1-2 hours prior to mid-log phase (OD₆₀₀˜0.5). 20 μL cells at exponential growth were labeled with 1 μL ALFA-NS (1 μM), then 2 μL cells were immobilized on 1×PBS pads with 2% wt/v agarose (Thermo-Fisher) and directly imaged with a with a Plan Apo Lambda DM 60X (1.4 NA, Ph3) oil objective. Images were analyzed and presented as mentioned above.

### Mass Spectrometry Methods

Excised gel bands were cut into approximately 1 mm³ pieces. Gel pieces were then subjected to a modified in-gel trypsin or chymotrypsin digestion procedure that included modification of cysteines with iodoacetamide.⁶ Gel pieces were washed and dehydrated with acetonitrile for 10 min. After the removal of the liquid phase, pieces were completely dried in a speed-vac. Rehydration of the gel pieces was with 50 mM ammonium bicarbonate solution containing 12.5 ng/μL modified sequencing-grade trypsin (Promega, Madison, WI) at 4° C. After 45 min, the excess trypsin solution was removed and replaced with 50 mM ammonium bicarbonate solution to cover the gel pieces. Samples were then placed at 37° C. overnight. Peptides were later extracted by removing the ammonium bicarbonate solution, followed by one wash with a solution containing 50% acetonitrile and 1% formic acid. The extracts were then dried in a speed-vac (˜1 h). The samples were stored at 4° C. until analysis.

On the day of analysis, the samples were reconstituted in 5-10 μL of HPLC solvent A (2.5% acetonitrile, 0.1% formic acid). A nano-scale reverse-phase HPLC capillary column was created by packing 2.6 μm C18 spherical silica beads into a fused silica capillary (100 μm inner diameter×˜30 cm length) with a flame-drawn tip⁷. After equilibrating the column each sample was loaded via a Famos auto sampler (LC Packings, San Francisco CA) onto the column. A gradient was formed, and peptides were eluted with increasing concentrations of solvent B (97.5% acetonitrile, 0.1% formic acid). As peptides eluted, they were subjected to electrospray ionization and then to an LTQ Orbitrap Velos Pro ion-trap mass spectrometer (Thermo Fisher Scientific, Waltham, MA). Peptides were detected, isolated, and fragmented to produce a tandem mass spectrum of specific fragment ions for each peptide. Peptide sequences (and hence protein identity) were determined by matching VHH72 protein with the acquired fragmentation pattern by the Sequest (Thermo Fisher Scientific, Waltham, MA) software.⁸ All databases include a reversed version of all the sequences and the data was filtered to between a one and two percent peptide false discovery rate.

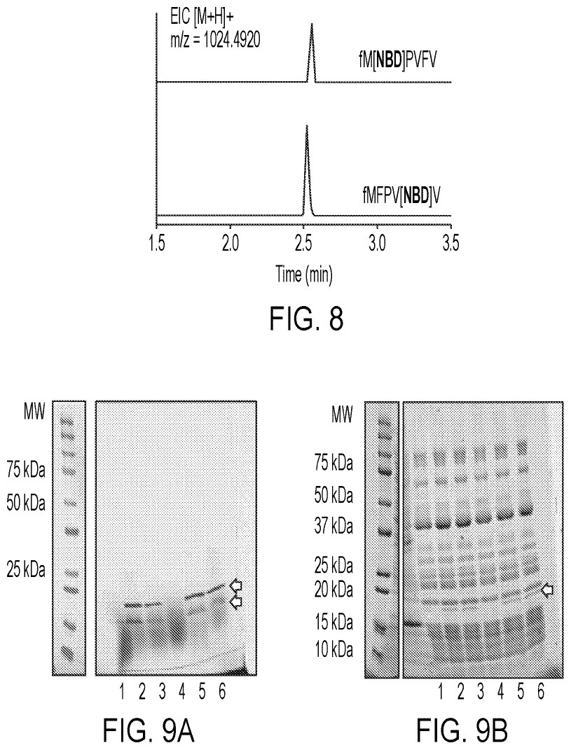

Data leading to FIG. 8 shows high resolution UPLC/MS analysis of NBDxK incorporated peptides. First, equal volume of 2% formic acid was added to PURE reactions to precipitate large proteins. Samples were then centrifuged at >12,000×g for 10 min. Samples in 1% formic acid (1 μL injection) were ran on an Agilent 1290 UPLC using a Poroshell 120 SB-Aq column (2.7 μm, 2.1×50 mm; Agilent) with a linear gradient from 5% to 100% acetonitrile over 3.5 min at a flow rate of 0.6 mL/min with 0.1% formic acid in the mobile phase. Mass spectra were acquired using an Agilent 6530c QTOF with the following source and acquisition parameters: Gas temperature=300° C.; gas flow=8 l/min; nebulizer=35 psig; capillary voltage=3500 V; fragmentor 175 V; skimmer 65 V; oct 1 RF vpp=750 V; acquisition rate=3 spectra/s; acquisition time=333.3 ms/spectrum; collision energy 0 V. Extracted ions for NT-formyl peptides (fM[NBD]PVFV and fMFPV[NBD]V; [M+H]+ m/z=1024.4920) were monitored within a 10 ppm window.

tRNA Ligation and Quantification

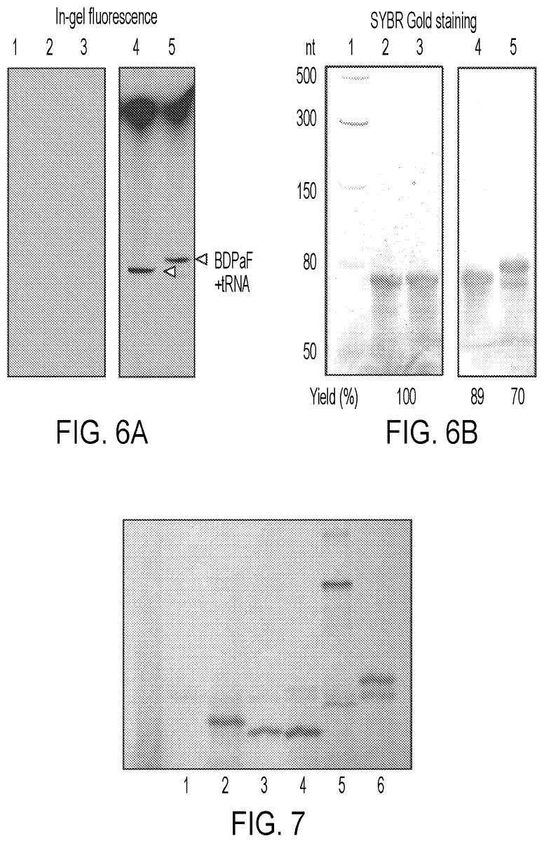

The enzymatic esterification of tRNA(-CA) species to nsAA-pdCpAs (resulting nsAA-tRNA_{CUA}) was done as previously described.¹ Briefly, 500 μg of PylT tRNA(-CA)_{CUA} or Mycoplasma capricolum Trp1 tRNA(-CA)_{CUA} was dissolved in 625 μL 10 mM HEPES+2.5 mM MgCl₂ and folded by heating to 95° C. for 3 min with a subsequent gradual cool-down to 25° C. over 20 min. The aminoacylation reaction to obtain the full length nsAA-tRNA CUA contained the final concentrations of 300 μg/mL folded tRNA(-CA), 0.3 mM nsAA-pdCpA (from 3 mM DMSO stock), 1× of T4 RNA Ligase buffer (from 10×, NEB), 0.125 mM ATP and 600 units/mL of T4 RNA Ligase 1 (NEB). This reaction was incubated at 4° C. for 2 h. The nsAA-tRNA CUA was extracted with acidic phenol chloroform (5:1, pH 4.5), ethanol precipitated, washed, air-dried and stored at −80° C. To determine aminoacylation efficiencies (FIG. 6) 1 μg of BDPaF-tRNA CUA was diluted in Novex™ TBE-Urea Sample Buffer (2×) and loaded onto a TBE urea gel (15%). Electrophoresis was carried out at 120 V for 4 hours in 1×TBE. The gel was then scanned for in gel fluorescence using a Fuji FLA-5100 fluorescent image analyser and subsequently stained with SYBR™ Gold Nucleic Acid Gel Stain and visualised under UV. Charging yield was calculated by quantifying band intensity on the UV scanned gel using FIJI.

### Cell-Free Transcription and Protein Translation and Quantification

DNA templates for the in vitro transcription and translation reactions contained optimized sequences of a T7 promoter, a Shine-Dalgarno sequence, the open reading frame with an in-frame amber stop codon (*=TAG) that would be suppressed in the presence of charged full-length tRNA species, e.g., NBDxK-tRNA_{CUA}. These templates were typically prepared as circular DNA. Exceptions were mRNA/cDNA display experiments and the experiments leading to FIG. 8, which relied on linear DNA recovered from the previous selection round, and Pep-MFPVFV, Pep-M*PVFV and Pep-PFPV*V sequences (Table 3), amplified by PriPep-F and PriPep-R primers (Table 5), respectively.

In vitro transcription/translation (IVTT) reactions were carried out using PURExpress® Δ RF1 or NEBExpress Cell-free E. coli Protein Synthesis System (NEB, Ipswich MA) following the manufacturer's instructions and supplying 20 ng/μL DNA templates with nsAA-tRNA_{CUA} (at final˜8 M), and 1.5 units/μL RNase Inhibitor Murine (NEB). In experiments where active nanosensors were ribosomally produced, the IVTT reactions were also supplemented with PURExpress® Disulfide Bond Enhancer (NEB). Reactions (5-50 μL) were incubated in 0.2 mL thin wall PCR tubes (Thermo Fisher) at 37° C. for 60 min-240 min. IVTT reactions were analyzed by running 1 μL of the reaction in parallel with 10 μL Precision Plus Protein™ fluorescent protein ladder (Bio-Rad) in 1.0 mm Invitrogen™ Novex™ WedgeWell™ 10-20%, Tris-Glycine mini protein gels (Thermo Fischer) following the manufacturer's instructions and materials. The in-gel fluorescence was measured using a Biorad Gel Doc XR+ Imaging System and the gels were Coomassie stained with InstantBlue protein stain (Novus Biologicals), following the manufacturer's instructions. The images were quantified by ImageJ.

For high-throughput screening of ribosomally produced nanosensor variants without purification (FIG. 12), 2.5 μL of IVTT reactions were mixed with equal volume of 1×PBS (negative control), RBD_{W}, or RBD_{OB.1} in PCR tubes and their fluorescence was quantified by a Biorad Gel Doc XR+ Imaging System and analyzed by ImageJ.

For real-time sensing of RBD_{W} by nascent Wuhan-NS (FIG. 3B), 5 μL of IVTT reactions were mixed with 0.25 μL 10 mM Tris at pH=7 (negative control) or concentrated RBD_{W} (exchanged to 10 mM Tris at final RBD_{W}˜2.5 μM) in low volume 384-well black flat clear bottom plates (Corning). Relative fluorescence units for NBDxK were recorded at excitation/emission wavelengths of 485 nm/528 nm using a Biotek spectrophotometric plate reader at 30° C. over 3 h. The signal values were normalized to peak fluorescence magnitude within an experiment and the graph was plotted indicating the standard deviation between repeats in shade. Graphs were plotted and analyzed in Prism 9 for Windows, GraphPad Software.

mRNA/cDNA Display

ORF libraries for mRNA/cDNA display were constructed step-wise. First, a VHH72 V104UAG library with randomized CDR2 and CDR3 was built by 4 cycles of overlap extension PCR with Pri1-Pri4 (acquired as PAGE purified Ultramers, IDT) that also allowed the representation of tryptophans in the library. For this, PCR reactions of Pri1-Pri2, Pri1-Pri4, Pri2-Pri3 and, Pri3-Pri4 were pooled in 1000:33:33:1 ratio followed by amplification with Pri5&Pri6 (5-10 cycles), and PCR purification (Table 5). This library insert sequence (˜125 ng) was assembled into the pPURExpress VHH72 V104UAG plasmid backbone (˜400 ng, linearized by the primers Pri7 and Pri8) in a 150 μL Gibson assembly reaction (NEB 50° C., 1 h). The product was then cleaned and concentrated by ethanol precipitation and the entire product was electro-transformed into ElectroMAX™ DH10B Cells (Thermo Fisher) cells. After cells were recovered in SOC for 1 h, overnight cultures were set up for plasmid minipreps by adding 4 mL 2×YT supplemented with carbenicillin (to final 100 μg mL-1) at 37° C. with aeration. In parallel, dilutions were plated to estimate the library size. 100 colonies were randomly selected and sequenced (Azenta/Genewiz) to estimate the library quality. This library was further diversified by one-piece isothermal assembly using the plasmid library backbone that is linearized by Pri9 & Pri10 and the same cloning strategy. This resulted in the library ORF, LibOmic, containing the fixed in-frame TAG stop codon at position 104 (Table 3 also shown in FIG. 4B).

The mRNA/cDNA display approach involves translating nsAA-containing nanobody libraries and covalently linking them to their cDNA via a puromycin linker. This is achieved by modifying a previously optimized protocol⁹ that includes a key new step that allows site-specific incorporation of nsAAs at the binding interface as described above (FIG. 10). The specific deviations from the protocol were the following: LibOmic was initially amplified with Pri11-Pri12 that added a 3′ T7 promoter followed by an optimized RBS and a 5′ His-tag followed by a flexible mRNA/cDNA display tag. After this step the linear DNA library for transcription was amplified by Pri11-Pri13. Alternatively, Pri14-Pri15 was used to amplify the DNA library for isothermal assembly cloning into pPURExpress backbone which is linearized by using Pri16-17. The IVTT reaction using PURExpress® Δ RF1 contained 8 M NBDxK-tRNA_{CUA} and was performed at 30° C. for 90 min. After reverse transcription, full-length nanosensors linked to their mRNA and cDNA were enriched via His-Pull-Down using 10 μL Pierce™ Ni-NTA Magnetic Agarose Beads and following the manufacturer's instructions. The elution (˜150 μL) was moved to the negative and positive selections, which were performed using 20 μL Magnetic Beads™ Streptavidin (from 1 mg/mL, Acro Biosystems, Cat. No. SMB-B01) and gradually decreasing amounts of SARS-CoV-2 (Omicron) Spike RBD-coupled Magnetic Beads (Acro Biosystems, Cat. No. MBS-K043), respectively. The final elution was done using streptavidin elution buffer (G-BIOSCIENCES, Cat. No. 786-549) followed by neutralization with equal volume of 1M Tris, pH 8 and ethanol precipitation. The pellet was reconstituted in water or either (i) used as the template for the next cycle by reamplifying with Pri11-Pri13, (ii) cloned into pPURExpress by amplifying with Pri14-Pri15 as mentioned above, or (iii) amplified by Pri5-Pri6 for next generation sequencing (Amplicon-EZ, Azenta/Genewiz).

### Illumina Next Generation Sequencing (NGS) Data Analysis and Read Counting

NGS-based amplicon sequencing was performed using Amplicon-EZ service of Azenta/Genewiz and DNA amplicons from each cycle (amplified by Pri5-Pri6) were prepared following Amplicon-EZ sample submission guidelines. Raw reads (236 bp pair-ended) were merged using BBMerge¹⁰, and filtered for Phred quality scores at or above 20. Resulting reads were forwarded and trimmed using a custom Python script, which identified the first 18 bp of the constant region prior to CDR2. Reads were trimmed such that the forward read started at CDR2 and extended through CDR3 to identify nanosensor variant combinations within both regions and counts of identical sequences were determined. The read frequency was calculated as the fraction of each unique sequence divided by the total number of trimmed sequences detected within a sample. Almost 200 unique reads were identified in all samples from the mRNA/cDNA display evolution rounds, and fold enrichment was calculated by dividing read frequencies of subsequent rounds by the read's frequency identified in the original library. Scripts are available upon request.

TABLE 3
Sequences of new constructs

| Name | SEQ ID NO. | Sequence |
|---|---|---|
| pET-28a (+) | 13 | AAAGCCCGAAAGGAAGCTGAGTTGGCTGCTGCCACCGCTGA |
| (backbone with | | GCAATAACTAGCATAACCCCTTGGGGCCTCTAAACGGGTCTT |
| N' His & | | GAGGGGTTTTTTGCTGAAAGGAGGAACTATATCCGGATTGGC |
| Thrombin | | GAATGGGACGCGCCCTGTAGCGGCGCATTAAGCGCGGCGGG |
| | | TGTGGTGGTTACGCGCAGCGTGACCGCTACACTTGCCAGCGC |
| | | CCTAGCGCCCGCTCCTTTCGCTTTCTTCCCTTCCTTTCTCGCCA |
| | | CGTTCGCCGGCTTTCCCCGTCAAGCTCTAAATCGGGGGCTCC |
| | | CTTTAGGGTTCCGATTTAGTGCTTTACGGCACCTCGACCCCAA |
| | | AAAACTTGATTAGGGTGATGGTTCACGTAGTGGGCCATCGCC |
| | | CTGATAGACGGTTTTTCGCCCTTTGACGTTGGAGTCCACGTTC |
| | | TTTAATAGTGGACTCTTGTTCCAAACTGGAACAACACTCAAC |
| | | CCTATCTCGGTCTATTCTTTTGATTTATAAGGGATTTTGCCGA |
| | | TTTCGGCCTATTGGTTAAAAAATGAGCTGATTTAACAAAAAT |
| | | TTAACGCGAATTTTAACAAAATATTAACGTTTACAATTTCAG |
| | | GTGGCACTTTTCGGGGAAATGTGCGCGGAACCCCTATTTGTT |
| | | TATTTTTCTAAATACATTCAAATATGTATCCGCTCATGAATTA |
| | | ATTCTTAGAAAAACTCATCGAGCATCAAATGAAACTGCAATT |
| | | TATTCATATCAGGATTATCAATACCATATTTTTGAAAAAGCC |
| | | GTTTCTGTAATGAAGGAGAAAACTCACCGAGGCAGTTCCATA |
| | | GGATGGCAAGATCCTGGTATCGGTCTGCGATTCCGACTCGTC |
| | | CAACATCAATACAACCTATTAATTTCCCCTCGTCAAAAATAA |
| | | GGTTATCAAGTGAGAAATCACCATGAGTGACGACTGAATCCG |
| | | GTGAGAATGGCAAAAGTTTATGCATTTCTTTCCAGACTTGTTC |
| | | AACAGGCCAGCCATTACGCTCGTCATCAAAATCACTCGCATC |
| | | AACCAAACCGTTATTCATTCGTGATTGCGCCTGAGCGAGACG |
| | | AAATACGCGATCGCTGTTAAAAGGACAATTACAAACAGGAA |
| | | TCGAATGCAACCGGCGCAGGAACACTGCCAGCGCATCAACA |
| | | ATATTTTCACCTGAATCAGGATATTCTTCTAATACCTGGAATG |
| | | CTGTTTTCCCGGGGATCGCAGTGGTGAGTAACCATGCATCAT |
| | | CAGGAGTACGGATAAAATGCTTGATGGTCGGAAGAGGCATA |
| | | AATTCCGTCAGCCAGTTTAGTCTGACCATCTCATCTGTAACAT |
| | | CATTGGCAACGCTACCTTTGCCATGTTTCAGAAACAACTCTG |
| | | GCGCATCGGGCTTCCCATACAATCGATAGATTGTCGCACCTG |
| | | ATTGCCCGACATTATCGCGAGCCCATTTATACCCATATAAAT |
| | | CAGCATCCATGTTGGAATTTAATCGCGGCCTAGAGCAAGACG |
| | | TTTCCCGTTGAATATGGCTCATAACACCCCTTGTATTACTGTT |
| | | TATGTAAGCAGACAGTTTTATTGTTCATGACCAAAATCCCTTA |
| | | ACGTGAGTTTTCGTTCCACTGAGCGTCAGACCCCGTAGAAAA |
| | | GATCAAAGGATCTTCTTGAGATCCTTTTTTTCTGCGCGTAATC |
| | | TGCTGCTTGCAAACAAAAAAACCACCGCTACCAGCGGTGGTT |
| | | TGTTTGCCGGATCAAGAGCTACCAACTCTTTTTCCGAAGGTA |
| | | ACTGGCTTCAGCAGAGCGCAGATACCAAATACTGTCCTTCTA |
| | | GTGTAGCCGTAGTTAGGCCACCACTTCAAGAACTCTGTAGCA |
| | | CCGCCTACATACCTCGCTCTGCTAATCCTGTTACCAGTGGCTG |
| | | CTGCCAGTGGCGATAAGTCGTGTCTTACCGGGTTGGACTCAA |
| | | GACGATAGTTACCGGATAAGGCGCAGCGGTCGGGCTGAACG |
| | | GGGGGTTCGTGCACACAGCCCAGCTTGGAGCGAACGACCTAC |
| | | ACCGAACTGAGATACCTACAGCGTGAGCTATGAGAAAGCGC |
| | | CACGCTTCCCGAAGGGAGAAAGGCGGACAGGTATCCGGTAA |
| | | GCGGCAGGGTCGGAACAGGAGAGCGCACGAGGGAGCTTCCA |
| | | GGGGGAAACGCCTGGTATCTTTATAGTCCTGTCGGGTTTCGC |
| | | CACCTCTGACTTGAGCGTCGATTTTTGTGATGCTCGTCAGGGG |
| | | GGCGGAGCCTATGGAAAAACGCCAGCAACGCGGCCTTTTTAC |
| | | GGTTCCTGGCCTTTTGCTGGCCTTTTGCTCACATGTTCTTTCCT |
| | | GCGTTATCCCCTGATTCTGTGGATAACCGTATTACCGCCTTTG |
| | | AGTGAGCTGATACCGCTCGCCGCAGCCGAACGACCGAGCGC |
| | | AGCGAGTCAGTGAGCGAGGAAGCGGAAGAGCGCCTGATGCG |
| | | GTATTTTCTCCTTACGCATCTGTGCGGTATTTCACACCGCATA |
| | | TATGGTGCACTCTCAGTACAATCTGCTCTGATGCCGCATAGTT |
| | | AAGCCAGTATACACTCCGCTATCGCTACGTGACTGGGTCATG |
| | | GCTGCGCCCCGACACCCGCCAACACCCGCTGACGCGCCCTGA |
| | | CGGGCTTGTCTGCTCCCGGCATCCGCTTACAGACAAGCTGTG |
| | | ACCGTCTCCGGGAGCTGCATGTGTCAGAGGTTTTCACCGTCA |
| | | TCACCGAAACGCGCGAGGCAGCTGCGGTAAAGCTCATCAGC |
| | | GTGGTCGTGAAGCGATTCACAGATGTCTGCCTGTTCATCCGC |
| | | GTCCAGCTCGTTGAGTTTCTCCAGAAGCGTTAATGTCTGGCTT |
| | | CTGATAAAGCGGGCCATGTTAAGGGCGGTTTTTTCCTGTTTG |
| | | GTCACTGATGCCTCCGTGTAAGGGGGATTTCTGTTCATGGGG |
| | | GTAATGATACCGATGAAACGAGAGAGGATGCTCACGATACG |
| | | GGTTACTGATGATGAACATGCCCGGTTACTGGAACGTTGTGA |
| | | GGGTAAACAACTGGCGGTATGGATGCGGCGGGACCAGAGAA |
| | | AAATCACTCAGGGTCAATGCCAGCGCTTCGTTAATACAGATG |
| | | TAGGTGTTCCACAGGGTAGCCAGCAGCATCCTGCGATGCAGA |
| | | TCCGGAACATAATGGTGCAGGGCGCTGACTTCCGCGTTTCCA |
| | | GACTTTACGAAACACGGAAACCGAAGACCATTCATGTTGTTG |
| | | CTCAGGTCGCAGACGTTTTGCAGCAGCAGTCGCTTCACGTTC |
| | | GCTCGCGTATCGGTGATTCATTCTGCTAACCAGTAAGGCAAC |
| | | CCCGCCAGCCTAGCCGGGTCCTCAACGACAGGAGCACGATCA |
| | | TGCGCACCCGTGGGGCCGCCATGCCGGCGATAATGGCCTGCT |
| | | TCTCGCCGAAACGTTTGGTGGCGGGACCAGTGACGAAGGCTT |
| | | GAGCGAGGGCGTGCAAGATTCCGAATACCGCAAGCGACAGG |
| | | CCGATCATCGTCGCGCTCCAGCGAAAGCGGTCCTCGCCGAAA |
| | | ATGACCCAGAGCGCTGCCGGCACCTGTCCTACGAGTTGCATG |
| | | ATAAAGAAGACAGTCATAAGTGCGGCGACGATAGTCATGCC |
| | | CCGCGCCCACCGGAAGGAGCTGACTGGGTTGAAGGCTCTCAA |
| | | GGGCATCGGTCGAGATCCCGGTGCCTAATGAGTGAGCTAACT |
| | | TACATTAATTGCGTTGCGCTCACTGCCCGCTTTCCAGTCGGGA |
| | | AACCTGTCGTGCCAGCTGCATTAATGAATCGGCCAACGCGCG |
| | | GGGAGAGGCGGTTTGCGTATTGGGCGCCAGGGTGGTTTTTCT |
| | | TTTCACCAGTGAGACGGGCAACAGCTGATTGCCCTTCACCGC |
| | | CTGGCCCTGAGAGAGTTGCAGCAAGCGGTCCACGCTGGTTTG |
| | | CCCCAGCAGGCGAAAATCCTGTTTGATGGTGGTTAACGGCGG |
| | | GATATAACATGAGCTGTCTTCGGTATCGTCGTATCCCACTACC |
| | | GAGATATCCGCACCAACGCGCAGCCCGGACTCGGTAATGGC |
| | | GCGCATTGCGCCCAGCGCCATCTGATCGTTGGCAACCAGCAT |
| | | CGCAGTGGGAACGATGCCCTCATTCAGCATTTGCATGGTTTG |
| | | TTGAAAACCGGACATGGCACTCCAGTCGCCTTCCCGTTCCGC |
| | | TATCGGCTGAATTTGATTGCGAGTGAGATATTTATGCCAGCC |
| | | AGCCAGACGCAGACGCGCCGAGACAGAACTTAATGGGCCCG |
| | | CTAACAGCGCGATTTGCTGGTGACCCAATGCGACCAGATGCT |
| | | CCACGCCCAGTCGCGTACCGTCTTCATGGGAGAAAATAATAC |
| | | TGTTGATGGGTGTCTGGTCAGAGACATCAAGAAATAACGCCG |
| | | GAACATTAGTGCAGGCAGCTTCCACAGCAATGGCATCCTGGT |
| | | CATCCAGCGGATAGTTAATGATCAGCCCACTGACGCGTTGCG |
| | | CGAGAAGATTGTGCACCGCCGCTTTACAGGCTTCGACGCCGC |
| | | TTCGTTCTACCATCGACACCACCACGCTGGCACCCAGTTGAT |
| | | CGGCGCGAGATTTAATCGCCGCGACAATTTGCGACGGCGCGT |
| | | GCAGGGCCAGACTGGAGGTGGCAACGCCAATCAGCAACGAC |
| | | TGTTTGCCCGCCAGTTGTTGTGCCACGCGGTTGGGAATGTAA |
| | | TTCAGCTCCGCCATCGCCGCTTCCACTTTTTCCCGCGTTTTCG |
| | | CAGAAACGTGGCTGGCCTGGTTCACCACGCGGGAAACGGTCT |
| | | GATAAGAGACACCGGCATACTCTGCGACATCGTATAACGTTA |
| | | CTGGTTTCACATTCACCACCCTGAATTGACTCTCTTCCGGGCG |
| | | CTATCATGCCATACCGCGAAAGGTTTTGCGCCATTCGATGGT |
| | | GTCCGGGATCTCGACGCTCTCCCTTATGCGACTCCTGCATTAG |
| | | GAAGCAGCCCAGTAGTAGGTTGAGGCCGTTGAGCACCGCCG |
| | | CCGCAAGGAATGGTGCATGCAAGGAGATGGCGCCCAACAGT |
| | | CCCCCGGCCACGGGGCCTGCCACCATACCCACGCCGAAACAA |
| | | GCGCTCATGAGCCCGAAGTGGCGAGCCCGATCTTCCCCATCG |
| | | GTGATGTCGGCGATATAGGCGCCAGCAACCGCACCTGTGGCG |
| | | CCGGTGATGCCGGCCACGATGCGTCCGGCGTAGAGGATCGA |
| | | GATCTCGATCCCGCGAAATTAATACGACTCACTATAGGGGAA |
| | | TTGTGAGCGGATAACAATTCCCCTCTAGAAATAATTTTGTTTA |
| | | ACTTTAAGAAGGAGATATACCATGGGCAGCAGCCATCATCAT |
| | | CATCATCACAGCAGCGGCCTGGTGCCGCGCGGCAGCCATTAA |
| | | CGAATTCGAGCTCCGTCGACAAGCTTGCGGCCGCACTCGAGC |
| | | ACCACCACCACCACCACTGAGATCCGGCTGCTAAC |
| pET-28_TEV | 14 | AAAGCCCGAAAGGAAGCTGAGTTGGCTGCTGCCACCGCTGA |
| (bb with N' His | | GCAATAACTAGCATAACCCCTTGGGGCCTCTAAACGGGTCTT |
| & TEV) | | GAGGGGTTTTTTGCTGAAAGGAGGAACTATATCCGGATTGGC |
| | | GAATGGGACGCGCCCTGTAGCGGCGCATTAAGCGCGGCGGG |
| | | TGTGGTGGTTACGCGCAGCGTGACCGCTACACTTGCCAGCGC |
| | | CCTAGCGCCCGCTCCTTTCGCTTTCTTCCCTTCCTTTCTCGCCA |
| | | CGTTCGCCGGCTTTCCCCGTCAAGCTCTAAATCGGGGGCTCC |
| | | CTTTAGGGTTCCGATTTAGTGCTTTACGGCACCTCGACCCCAA |
| | | AAAACTTGATTAGGGTGATGGTTCACGTAGTGGGCCATCGCC |
| | | CTGATAGACGGTTTTTCGCCCTTTGACGTTGGAGTCCACGTTC |
| | | TTTAATAGTGGACTCTTGTTCCAAACTGGAACAACACTCAAC |
| | | CCTATCTCGGTCTATTCTTTTGATTTATAAGGGATTTTGCCGA |
| | | TTTCGGCCTATTGGTTAAAAAATGAGCTGATTTAACAAAAAT |
| | | TTAACGCGAATTTTAACAAAATATTAACGTTTACAATTTCAG |
| | | GTGGCACTTTTCGGGGAAATGTGCGCGGAACCCCTATTTGTT |
| | | TATTTTTCTAAATACATTCAAATATGTATCCGCTCATGAATTA |
| | | ATTCTTAGAAAAACTCATCGAGCATCAAATGAAACTGCAATT |
| | | TATTCATATCAGGATTATCAATACCATATTTTTGAAAAAGCC |
| | | GTTTCTGTAATGAAGGAGAAAACTCACCGAGGCAGTTCCATA |
| | | GGATGGCAAGATCCTGGTATCGGTCTGCGATTCCGACTCGTC |
| | | CAACATCAATACAACCTATTAATTTCCCCTCGTCAAAAATAA |
| | | GGTTATCAAGTGAGAAATCACCATGAGTGACGACTGAATCCG |
| | | GTGAGAATGGCAAAAGTTTATGCATTTCTTTCCAGACTTGTTC |
| | | AACAGGCCAGCCATTACGCTCGTCATCAAAATCACTCGCATC |
| | | AACCAAACCGTTATTCATTCGTGATTGCGCCTGAGCGAGACG |
| | | AAATACGCGATCGCTGTTAAAAGGACAATTACAAACAGGAA |
| | | TCGAATGCAACCGGCGCAGGAACACTGCCAGCGCATCAACA |
| | | ATATTTTCACCTGAATCAGGATATTCTTCTAATACCTGGAATG |
| | | CTGTTTTCCCGGGGATCGCAGTGGTGAGTAACCATGCATCAT |
| | | CAGGAGTACGGATAAAATGCTTGATGGTCGGAAGAGGCATA |
| | | AATTCCGTCAGCCAGTTTAGTCTGACCATCTCATCTGTAACAT |
| | | CATTGGCAACGCTACCTTTGCCATGTTTCAGAAACAACTCTG |
| | | GCGCATCGGGCTTCCCATACAATCGATAGATTGTCGCACCTG |
| | | ATTGCCCGACATTATCGCGAGCCCATTTATACCCATATAAAT |
| | | CAGCATCCATGTTGGAATTTAATCGCGGCCTAGAGCAAGACG |
| | | TTTCCCGTTGAATATGGCTCATAACACCCCTTGTATTACTGTT |
| | | TATGTAAGCAGACAGTTTTATTGTTCATGACCAAAATCCCTTA |
| | | ACGTGAGTTTTCGTTCCACTGAGCGTCAGACCCCGTAGAAAA |
| | | GATCAAAGGATCTTCTTGAGATCCTTTTTTTCTGCGCGTAATC |
| | | TGCTGCTTGCAAACAAAAAAACCACCGCTACCAGCGGTGGTT |
| | | TGTTTGCCGGATCAAGAGCTACCAACTCTTTTTCCGAAGGTA |
| | | ACTGGCTTCAGCAGAGCGCAGATACCAAATACTGTCCTTCTA |
| | | GTGTAGCCGTAGTTAGGCCACCACTTCAAGAACTCTGTAGCA |
| | | CCGCCTACATACCTCGCTCTGCTAATCCTGTTACCAGTGGCTG |
| | | CTGCCAGTGGCGATAAGTCGTGTCTTACCGGGTTGGACTCAA |
| | | GACGATAGTTACCGGATAAGGCGCAGCGGTCGGGCTGAACG |
| | | GGGGGTTCGTGCACACAGCCCAGCTTGGAGCGAACGACCTAC |
| | | ACCGAACTGAGATACCTACAGCGTGAGCTATGAGAAAGCGC |
| | | CACGCTTCCCGAAGGGAGAAAGGCGGACAGGTATCCGGTAA |
| | | GCGGCAGGGTCGGAACAGGAGAGCGCACGAGGGAGCTTCCA |
| | | GGGGGAAACGCCTGGTATCTTTATAGTCCTGTCGGGTTTCGC |
| | | CACCTCTGACTTGAGCGTCGATTTTTGTGATGCTCGTCAGGGG |
| | | GGCGGAGCCTATGGAAAAACGCCAGCAACGCGGCCTTTTTAC |
| | | GGTTCCTGGCCTTTTGCTGGCCTTTTGCTCACATGTTCTTTCCT |
| | | GCGTTATCCCCTGATTCTGTGGATAACCGTATTACCGCCTTTG |
| | | AGTGAGCTGATACCGCTCGCCGCAGCCGAACGACCGAGCGC |
| | | AGCGAGTCAGTGAGCGAGGAAGCGGAAGAGCGCCTGATGCG |
| | | GTATTTTCTCCTTACGCATCTGTGCGGTATTTCACACCGCATA |
| | | TATGGTGCACTCTCAGTACAATCTGCTCTGATGCCGCATAGTT |
| | | AAGCCAGTATACACTCCGCTATCGCTACGTGACTGGGTCATG |
| | | GCTGCGCCCCGACACCCGCCAACACCCGCTGACGCGCCCTGA |
| | | CGGGCTTGTCTGCTCCCGGCATCCGCTTACAGACAAGCTGTG |
| | | ACCGTCTCCGGGAGCTGCATGTGTCAGAGGTTTTCACCGTCA |
| | | TCACCGAAACGCGCGAGGCAGCTGCGGTAAAGCTCATCAGC |
| | | GTGGTCGTGAAGCGATTCACAGATGTCTGCCTGTTCATCCGC |
| | | GTCCAGCTCGTTGAGTTTCTCCAGAAGCGTTAATGTCTGGCTT |
| | | CTGATAAAGCGGGCCATGTTAAGGGCGGTTTTTTCCTGTTTG |
| | | GTCACTGATGCCTCCGTGTAAGGGGGATTTCTGTTCATGGGG |
| | | GTAATGATACCGATGAAACGAGAGAGGATGCTCACGATACG |
| | | GGTTACTGATGATGAACATGCCCGGTTACTGGAACGTTGTGA |
| | | GGGTAAACAACTGGCGGTATGGATGCGGCGGGACCAGAGAA |
| | | AAATCACTCAGGGTCAATGCCAGCGCTTCGTTAATACAGATG |
| | | TAGGTGTTCCACAGGGTAGCCAGCAGCATCCTGCGATGCAGA |
| | | TCCGGAACATAATGGTGCAGGGCGCTGACTTCCGCGTTTCCA |
| | | GACTTTACGAAACACGGAAACCGAAGACCATTCATGTTGTTG |
| | | CTCAGGTCGCAGACGTTTTGCAGCAGCAGTCGCTTCACGTTC |
| | | GCTCGCGTATCGGTGATTCATTCTGCTAACCAGTAAGGCAAC |
| | | CCCGCCAGCCTAGCCGGGTCCTCAACGACAGGAGCACGATCA |
| | | TGCGCACCCGTGGGGCCGCCATGCCGGCGATAATGGCCTGCT |
| | | TCTCGCCGAAACGTTTGGTGGCGGGACCAGTGACGAAGGCTT |
| | | GAGCGAGGGCGTGCAAGATTCCGAATACCGCAAGCGACAGG |
| | | CCGATCATCGTCGCGCTCCAGCGAAAGCGGTCCTCGCCGAAA |
| | | ATGACCCAGAGCGCTGCCGGCACCTGTCCTACGAGTTGCATG |
| | | ATAAAGAAGACAGTCATAAGTGCGGCGACGATAGTCATGCC |
| | | CCGCGCCCACCGGAAGGAGCTGACTGGGTTGAAGGCTCTCAA |
| | | GGGCATCGGTCGAGATCCCGGTGCCTAATGAGTGAGCTAACT |
| | | TACATTAATTGCGTTGCGCTCACTGCCCGCTTTCCAGTCGGGA |
| | | AACCTGTCGTGCCAGCTGCATTAATGAATCGGCCAACGCGCG |
| | | GGGAGAGGCGGTTTGCGTATTGGGCGCCAGGGTGGTTTTTCT |
| | | TTTCACCAGTGAGACGGGCAACAGCTGATTGCCCTTCACCGC |
| | | CTGGCCCTGAGAGAGTTGCAGCAAGCGGTCCACGCTGGTTTG |
| | | CCCCAGCAGGCGAAAATCCTGTTTGATGGTGGTTAACGGCGG |
| | | GATATAACATGAGCTGTCTTCGGTATCGTCGTATCCCACTACC |
| | | GAGATATCCGCACCAACGCGCAGCCCGGACTCGGTAATGGC |
| | | GCGCATTGCGCCCAGCGCCATCTGATCGTTGGCAACCAGCAT |
| | | CGCAGTGGGAACGATGCCCTCATTCAGCATTTGCATGGTTTG |
| | | TTGAAAACCGGACATGGCACTCCAGTCGCCTTCCCGTTCCGC |
| | | TATCGGCTGAATTTGATTGCGAGTGAGATATTTATGCCAGCC |
| | | AGCCAGACGCAGACGCGCCGAGACAGAACTTAATGGGCCCG |
| | | CTAACAGCGCGATTTGCTGGTGACCCAATGCGACCAGATGCT |
| | | CCACGCCCAGTCGCGTACCGTCTTCATGGGAGAAAATAATAC |
| | | TGTTGATGGGTGTCTGGTCAGAGACATCAAGAAATAACGCCG |
| | | GAACATTAGTGCAGGCAGCTTCCACAGCAATGGCATCCTGGT |
| | | CATCCAGCGGATAGTTAATGATCAGCCCACTGACGCGTTGCG |
| | | CGAGAAGATTGTGCACCGCCGCTTTACAGGCTTCGACGCCGC |
| | | TTCGTTCTACCATCGACACCACCACGCTGGCACCCAGTTGAT |
| | | CGGCGCGAGATTTAATCGCCGCGACAATTTGCGACGGCGCGT |
| | | GCAGGGCCAGACTGGAGGTGGCAACGCCAATCAGCAACGAC |
| | | TGTTTGCCCGCCAGTTGTTGTGCCACGCGGTTGGGAATGTAA |
| | | TTCAGCTCCGCCATCGCCGCTTCCACTTTTTCCCGCGTTTTCG |
| | | CAGAAACGTGGCTGGCCTGGTTCACCACGCGGGAAACGGTCT |
| | | GATAAGAGACACCGGCATACTCTGCGACATCGTATAACGTTA |
| | | CTGGTTTCACATTCACCACCCTGAATTGACTCTCTTCCGGGCG |
| | | CTATCATGCCATACCGCGAAAGGTTTTGCGCCATTCGATGGT |
| | | GTCCGGGATCTCGACGCTCTCCCTTATGCGACTCCTGCATTAG |
| | | GAAGCAGCCCAGTAGTAGGTTGAGGCCGTTGAGCACCGCCG |
| | | CCGCAAGGAATGGTGCATGCAAGGAGATGGCGCCCAACAGT |
| | | CCCCCGGCCACGGGGCCTGCCACCATACCCACGCCGAAACAA |
| | | GCGCTCATGAGCCCGAAGTGGCGAGCCCGATCTTCCCCATCG |
| | | GTGATGTCGGCGATATAGGCGCCAGCAACCGCACCTGTGGCG |
| | | CCGGTGATGCCGGCCACGATGCGTCCGGCGTAGAGGATCGA |
| | | GATCTCGATCCCGCGAAATTAATACGACTCACTATAGGGGAA |
| | | TTGTGAGCGGATAACAATTCCCCTCTAGAAATAATTTTGTTTA |
| | | ACTTTAAGAAGGAGATATACCATGCATCACCATCATCATCAT |
| | | CATCACTCGTCAGGCGAGAATCTTTATTTTCAGAGCAGTGGT |
| | | TAATAACGAATTCGAGCTCCGTCGACAAGCTTGCGGCCGCAC |
| | | TCGAGCACCACCACCACCACCACTGAGATCCGGCTGCTAAC |
| pPURExpress | 15 | GCTAGTGGTGCTAGCCCCGCGAAATTAATACGACTCACTATA |
| (bb without a | | GGGTCTAGAAATAATTTTGTTTAACTTTAAGAAGGAGATATA |
| tag) | | CATATAATGAGGATCCCGGGAATTCTCGAGTAAGGTTAACCT |
| | | GCAGGAGGCCTTTAATTAAGGTGGTGCGGCCGCGCTAGCGGT |
| | | CCCGGGGGATCGATCCGGCTGCTAACAAAGCCCGAAAGGAA |
| | | GCTGAGTTGGCTGCTGCCACCGCTGAGCAATAACTAGCATAA |
| | | CCCCTTGGGGCCTCTAAACGGGTCTTGAGGGGTTTTTTGCTGA |
| | | AAGGAGGAACTATATCCGGAAGCTTGGCACTGGCCGACCGG |
| | | GGTCGAGCACTGACTCGCTGCGCTCGGTCGTTCGGCTGCGGC |
| | | GAGCGGTATCAGCTCACTCAAAGGCGGTAATACGGTTATCCA |
| | | CAGAATCAGGGGATAACGCAGGAAAGAACATGTGAGCAAAA |
| | | GGCCAGCAAAAGGCCAGGAACCGTAAAAAGGCCGCGTTGCT |
| | | GGCGTTTTTCCATAGGCTCCGCCCCCCTGACGAGCATCACAA |
| | | AAATCGACGCTCAAGTCAGAGGTGGCGAAACCCGACAGGAC |
| | | TATAAAGATACCAGGCGTTTCCCCCTGGAAGCTCCCTCGTGC |
| | | GCTCTCCTGTTCCGACCCTGCCGCTTACCGGATACCTGTCCGC |
| | | CTTTCTCCCTTCGGGAAGCGTGGCGCTTTCTCATAGCTCACGC |
| | | TGTAGGTATCTCAGTTCGGTGTAGGTCGTTCGCTCCAAGCTG |
| | | GGCTGTGTGCACGAACCCCCCGTTCAGCCCGACCGCTGCGCC |
| | | TTATCCGGTAACTATCGTCTTGAGTCCAACCCGCTAAGACAC |
| | | GACTTATCGCCACTGGCAGCAGCCACTGGTAACAGGATTAGC |
| | | AGAGCGAGGTATGTAGGCGGTGCTACAGAGTTCTTGAAGTGG |
| | | TGGCCTAACTACGGCTACACTAGAAGAACAGTATTTGGTATC |
| | | TGCGCTCTGCTGAAGCCAGTTACCTTCGGAAAAAGAGTTGGT |
| | | AGCTCTTGATCCGGCAAACAAACCACCGCTGGTAGCGGTGGT |
| | | TTTTTTGTTTGCAAGCAGCAGATTACGCGCAGAAAAAAAGGA |
| | | TCTCAAGAAGATCCTTTGATCTTTTCTACGGGGTCTGACGCTC |
| | | AGTGGAACGAAAACTCACAGATCCGGGATTTTGGTCATGAGA |
| | | TTATCAAAAAGGATCTTCACCTAGATCCTTTTAAATTAAAAA |
| | | TGAAGTTTTAAATCAATCTAAAGTATATATGAGTAAACTTGG |
| | | TCTGACAGTTACCAATGCTTAATCAGTGAGGCACCTATCTCA |
| | | GCGATCTGTCTATTTCGTTCATCCATAGTTGCCTGACTCCCCG |
| | | TCGTGTAGATAACTACGATACGGGAGGGCTTACCATCTGGCC |
| | | CCAGTGCTGCAATGATACCGCGAGACCCACGCTCACCGGCTC |
| | | CAGATTTATCAGCAATAAACCAGCCAGCCGGAAGGGCCGAG |
| | | CGCAGAAGTGGTCCTGCAACTTTATCCGCCTCCATCCAGTCT |
| | | ATTAATTGTTGCCGGGAAGCTAGAGTAAGTAGTTCGCCAGTT |
| | | AATAGTTTGCGCAACGTTGTTGCCATTGCTACAGGCATCGTG |
| | | GTGTCACGCTCGTCGTTTGGTATGGCTTCATTCAGCTCCGGTT |
| | | CCCAACGATCAAGGCGAGTTACATGATCCCCCATGTTGTGCA |
| | | AAAAAGCGGTTAGCTCCTTCGGTCCTCCGATCGTTGTCAGAA |
| | | GTAAGTTGGCCGCAGTGTTATCACTCATGGTTATGGCAGCAC |
| | | TGCATAATTCTCTTACTGTCATGCCATCCGTAAGATGCTTTTC |
| | | TGTGACTGGTGAGTACTCAACCAAGTCATTCTGAGAATAGTG |
| | | TATGCGGCGACCGAGTTGCTCTTGCCCGGCGTCAATACGGGA |
| | | TAATACCGCGCCACATAGCAGAACTTTAAAAGTGCTCATCAT |
| | | TGGAAAACGTTCTTCGGGGCGAAAACTCTCAAGGATCTTACC |
| | | GCTGTTGAGATCCAGTTCGATGTAACCCACTCGTGCACCCAA |
| | | CTGATCTTCAGCATCTTTTACTTTCACCAGCGTTTCTGGGTGA |
| | | GCAAAAACAGGAAGGCAAAATGCCGCAAAAAAGGGAATAA |
| | | GGGCGACACGGAAATGTTGAATACTCATACTCTTCCTTTTTCA |
| | | ATATTATTGAAGCATTTATCAGGGTTATTGTCTCATGAGCGG |
| | | ATACATATTTGAATGTATTTAGAAAAATAAACAAATAGGGGT |
| | | TCCGCGCACATTTCCCCGAAAAGT |
| pPURExpress | 16 | GCTAGTGGTGCTAGCCCCGCGAAATTAATACGACTCACTATA |
| VHH72 | | GGGTCTAGAAATAATTTTGTTTAACTTTAAGAAGGAGATATA |
| V104UAG | | CATATGCAAGTGCAGTTACAAGAGTCGGGTGGTGGACTGGTC |
| | | CAGGCCGGTGGCTCTTTGCGCTTAAGTTGTGCCGCTTCTGGCC |
| | | GTACTTTTTCAGAATACGCAATGGGATGGTTCCGTCAAGCTC |
| | | CGGGGAAAGAACGTGAGTTTGTGGCGACTATTTCCTGGTCTG |
| | | GGGGGTCTACCTACTACACAGACAGCGTAAAAGGCCGTTTCA |
| | | CAATCTCGCGCGATAACGCGAAGAATACCGTGTATCTGCAAA |
| | | TGAATTCATTGAAACCCGACGACACCGCAGTATATTATTGTG |
| | | CAGCGGCGGGGTTGGGTACGTAGGTTTCTGAGTGGGATTACG |
| | | ATTACGATTACTGGGGCCAGGGTACTCAAGTCACCGTAAGCT |
| | | CAGGATCTGGTGGTAGTGGAGGAGGTTCTGGTGGTGGTTCTG |
| | | GTTAATGAGGATCCCGGGAATTCTCGAGTAAGGTTAACCTGC |
| | | AGGAGGCCTTTAATTAAGGTGGTGCGGCCGCGCTAGCGGTCC |
| | | CGGGGGATCGATCCGGCTGCTAACAAAGCCCGAAAGGAAGC |
| | | TGAGTTGGCTGCTGCCACCGCTGAGCAATAACTAGCATAACC |
| | | CCTTGGGGCCTCTAAACGGGTCTTGAGGGGTTTTTTGCTGAA |
| | | AGGAGGAACTATATCCGGAAGCTTGGCACTGGCCGACCGGG |
| | | GTCGAGCACTGACTCGCTGCGCTCGGTCGTTCGGCTGCGGCG |
| | | AGCGGTATCAGCTCACTCAAAGGCGGTAATACGGTTATCCAC |
| | | AGAATCAGGGGATAACGCAGGAAAGAACATGTGAGCAAAAG |
| | | GCCAGCAAAAGGCCAGGAACCGTAAAAAGGCCGCGTTGCTG |
| | | GCGTTTTTCCATAGGCTCCGCCCCCCTGACGAGCATCACAAA |
| | | AATCGACGCTCAAGTCAGAGGTGGCGAAACCCGACAGGACT |
| | | ATAAAGATACCAGGCGTTTCCCCCTGGAAGCTCCCTCGTGCG |
| | | CTCTCCTGTTCCGACCCTGCCGCTTACCGGATACCTGTCCGCC |
| | | TTTCTCCCTTCGGGAAGCGTGGCGCTTTCTCATAGCTCACGCT |
| | | GTAGGTATCTCAGTTCGGTGTAGGTCGTTCGCTCCAAGCTGG |
| | | GCTGTGTGCACGAACCCCCCGTTCAGCCCGACCGCTGCGCCT |
| | | TATCCGGTAACTATCGTCTTGAGTCCAACCCGCTAAGACACG |
| | | ACTTATCGCCACTGGCAGCAGCCACTGGTAACAGGATTAGCA |
| | | GAGCGAGGTATGTAGGCGGTGCTACAGAGTTCTTGAAGTGGT |
| | | GGCCTAACTACGGCTACACTAGAAGAACAGTATTTGGTATCT |
| | | GCGCTCTGCTGAAGCCAGTTACCTTCGGAAAAAGAGTTGGTA |
| | | GCTCTTGATCCGGCAAACAAACCACCGCTGGTAGCGGTGGTT |
| | | TTTTTGTTTGCAAGCAGCAGATTACGCGCAGAAAAAAAGGAT |
| | | CTCAAGAAGATCCTTTGATCTTTTCTACGGGGTCTGACGCTCA |
| | | GTGGAACGAAAACTCACAGATCCGGGATTTTGGTCATGAGAT |
| | | TATCAAAAAGGATCTTCACCTAGATCCTTTTAAATTAAAAAT |
| | | GAAGTTTTAAATCAATCTAAAGTATATATGAGTAAACTTGGT |
| | | CTGACAGTTACCAATGCTTAATCAGTGAGGCACCTATCTCAG |
| | | CGATCTGTCTATTTCGTTCATCCATAGTTGCCTGACTCCCCGT |
| | | CGTGTAGATAACTACGATACGGGAGGGCTTACCATCTGGCCC |
| | | CAGTGCTGCAATGATACCGCGAGACCCACGCTCACCGGCTCC |
| | | AGATTTATCAGCAATAAACCAGCCAGCCGGAAGGGCCGAGC |
| | | GCAGAAGTGGTCCTGCAACTTTATCCGCCTCCATCCAGTCTAT |
| | | TAATTGTTGCCGGGAAGCTAGAGTAAGTAGTTCGCCAGTTAA |
| | | TAGTTTGCGCAACGTTGTTGCCATTGCTACAGGCATCGTGGT |
| | | GTCACGCTCGTCGTTTGGTATGGCTTCATTCAGCTCCGGTTCC |
| | | CAACGATCAAGGCGAGTTACATGATCCCCCATGTTGTGCAAA |
| | | AAAGCGGTTAGCTCCTTCGGTCCTCCGATCGTTGTCAGAAGT |
| | | AAGTTGGCCGCAGTGTTATCACTCATGGTTATGGCAGCACTG |
| | | CATAATTCTCTTACTGTCATGCCATCCGTAAGATGCTTTTCTG |
| | | TGACTGGTGAGTACTCAACCAAGTCATTCTGAGAATAGTGTA |
| | | TGCGGCGACCGAGTTGCTCTTGCCCGGCGTCAATACGGGATA |
| | | ATACCGCGCCACATAGCAGAACTTTAAAAGTGCTCATCATTG |
| | | GAAAACGTTCTTCGGGGCGAAAACTCTCAAGGATCTTACCGC |
| | | TGTTGAGATCCAGTTCGATGTAACCCACTCGTGCACCCAACT |
| | | GATCTTCAGCATCTTTTACTTTCACCAGCGTTTCTGGGTGAGC |
| | | AAAAACAGGAAGGCAAAATGCCGCAAAAAAGGGAATAAGG |
| | | GCGACACGGAAATGTTGAATACTCATACTCTTCCTTTTTCAAT |
| | | ATTATTGAAGCATTTATCAGGGTTATTGTCTCATGAGCGGAT |
| | | ACATATTTGAATGTATTTAGAAAAATAAACAAATAGGGGTTC |
| | | CGCGCACATTTCCCCGAAAAGT |
| LibOmic | 17 | ATGCAAGTGCAGTTACAAGAGTCGGGTGGTGGACTGGTCCAG |
| | | GCCGGTGGCTCTTTGCGCTTAAGTTGTGCCGCTTCTGGCCGTA |
| | | CTTTTTCAGAATACGCAATGGGATGGTTCCGTCAAGCTCCGG |
| | | GGCGTGAACGTGAGTTTGTGGCGACTATTNDTNSGTCTGGCG |
| | | GCNDTACCNDTTACACAGACAGCGTACGCGGCCGTTTCACAA |
| | | TCTCGCGCGATAACGCGCGTAATACCGTGTATCTGCAAATGA |
| | | ATTCATTGCGTCCCGACGACACCGCAGTATATTATTGTGCAG |
| | | CGGCGGGGTTGGGTNNCTAGNNCTCTGAGNNCGATNNCGATT |
| | | ACGATTACTGGGGCCAGGGTACTCAAGTCACCGTAAGCTCAG |
| | | GATCTGGTGGTCACCATCACCACCATCATGGAAGCGGCCATC |
| | | ACCATCATCATCATGGTTCTGGTGGTGGTTCTGGT |
| pDule-MjCouRS | 18 | CGCCGGTTAAGGCTAAACTGAAAGGACAAGTTTTGGTGACTG |
| (includes a | | CGCTCCTCCAAGCCAGTTACCTCGGTTCAAAGAGTTGGTAGC |
| copy of | | TCAGAGAACCTTCGAAAAACCGCCCTGCAAGGCGGTTTTTTC |
| tRNAcura gene) | | GTTTTCAGAGCAAGAGATTACGCGCAGACCAAAACGATCTCA |
| | | AGAAGATCATCTTATTAATCAGATAAAATATTTCTAGATTTC |
| | | AGTGCAATTTATCTCTTCAAATGTAGCACCTGAAGTCAGCCC |
| | | CATACGATATAAGTTGTAATTCTCATGTTTGACAGCTTATCAT |
| | | CGATAAGCTTTAATGCGGTAGTTTATCACAGTTAAATTGCTA |
| | | ACGCAGTCAGGCACCGTGTATGAAATCTAACAATGCGCTCAT |
| | | CGTCATCCTCGGCACCGTCACCCTGGATGCTGTAGGCATAGG |
| | | CTTGGTTATGCCGGTACTGCCGGGCCTCTTGCGGGATATCGTC |
| | | CATTCCGACAGCATCGCCAGTCACTATGGCGTGCTGCTAGCG |
| | | CTATATGCGTTGATGCAATTTCTATGCGCACCCGTTCTCGGAG |
| | | CACTGTCCGACCGCTTTGGCCGCCGCCCAGTCCTGCTCGCTTC |
| | | GCTACTTGGAGCCACTATCGACTACGCGATCATGGCGACCAC |
| | | ACCCGTCCTGTGGATCCTCTACGCCGGACGCATCGTGGCCGG |
| | | CATCACCGGCGCCACAGGTGCGGTTGCTGGCGCCTATATCGC |
| | | CGACATCACCGATGGGGAAGATCGGGCTCGCCACTTCGGGCT |
| | | CATGAGCGCTTGTTTCGGCGTGGGTATGGTGGCAGGCCCCGT |
| | | GGCCGGGGGACTGTTGGGCGCCATCTCCTTGCATGCACCATT |
| | | CCTTGCGGCGGCGGTGCTCAACGGCCTCAACCTACTACTGGG |
| | | CTGCTTCCTAATGCAGGAGTCGCATAAGGGAGAGCGTCGACC |
| | | GATGCCCTTGAGAGCCTTCAACCCAGTCAGCTCCTTCCGGTG |
| | | GGCGCGGGGCATGACTATCGTCGCCGCACTTATGACTGTCTT |
| | | CTTTATCATGCAACTCGTAGGACAGGTGCCGGCAGCGCTCTG |
| | | GGTCATTTTCGGCGAGGACCGCTTTCGCTGGAGCGCGACGAT |
| | | GATCGGCCTGTCGCTTGCGGTATTCGGAATCTTGCACGCCCTC |
| | | GCTCAAGCCTTCGTCACTGGTCCCGCCACCAAACGTTTCGGC |
| | | GAGAAGCAGGCCATTATCGCCGGCATGGCGGCCGACGCACT |
| | | GGGCTACGTCTTGCTGGCGTTCGCGACGCGAGGCTGGATGGC |
| | | CTTCCCCATTATGATTCTTCTCGCTTCCGGCGGCATCGGGATG |
| | | CCCGCGTTGCAGGCCATGCTGTCCAGGCAGGTAGATGACGAC |
| | | CATCAGGGACAGCTTCAAGGATCGCTCGCGGCTCTTACCAGC |
| | | CTAACTTCGATCATTGGACCGCTGATCGTCACGGCGATTTAT |
| | | GCCGCCTCGGCGAGCACATGGAACGGGTTGGCATGGATTGTA |
| | | GGCGCCGCCCTATACCTTGTCTGCCTCCCCGCGTTGCGTCGCG |
| | | GTGCATGGAGCCGGGCCACCTCGACCTGAATGGAAGCCGGC |
| | | GGCACCTCGCTAACGGATTCACCACTCCAAGAATTGGAGCCA |
| | | ATCAATTCTTGCGGAGAACTGTGAATGCGCAAACCAACCCTT |
| | | GGCAGAACATATCCATCGCGTCCGTATAATATCATACGCTGT |
| | | TATACGTTGTTTACGCTTTGAGGAATCCCATATGGACGAATTC |
| | | GAAATGATCAAACGTAACACCTCTGAAATCATCTCTGAAGAA |
| | | GAACTGCGTGAAGTTCTGAAAAAAGACGAAAAATCTGCGGA |
| | | AATCGGTTTCGAACCGTCTGGTAAAATCCACCTGGGTCACTA |
| | | CCTGCAGATCAAAAAAATGATCGACCTGCAGAACGCGGGTTT |
| | | CGACATCATCATCCATCTGGGTGACCTGGGAGCGTACCTGAA |
| | | CCAGAAAGGTGAACTGGACGAAATCCGTAAAATCGGTGACT |
| | | ACAACAAAAAAGTTTTCGAAGCGATGGGTCTGAAAGCGAAA |
| | | TACGTTTACGGTTCTGAATATCATCTGGACAAAGACTACACC |
| | | CTGAACGTTTACCGTCTGGCGCTGAAAACCACCCTGAAACGT |
| | | GCGCGTCGTTCTATGGAACTGATCGCGCGTGAAGACGAAAAC |
| | | CCGAAAGTTGCGGAAGTTATCTACCCGATCATGCAGGTTAAC |
| | | GGTATCCACTACGGTGGTGTTGACGTTGCGGTTGGTGGTATG |
| | | GAACAGCGTAAAATCCACATGCTGGCGCGTGAACTGCTGCCG |
| | | AAAAAAGTTGTTTGCATCCACAACCCGGTTCTGACCGGTCTG |
| | | GACGGTGAAGGTAAAATGTCTTCTTCTAAAGGTAACTTCATC |
| | | GCGGTTGACGACTCTCCGGAAGAAATCCGTGCGAAAATCAA |
| | | AAAAGCGTACTGCCCGGCAGGTGTTGTTGAAGGTAACCCGAT |
| | | CATGGAAATCGCGAAATACTTCCTGGAATACCCGCTGACCAT |
| | | CAAACGTCCGGAAAAATTCGGTGGTGACCTGACCGTTAACTC |
| | | TTACGAAGAACTGGAATCTCTGTTCAAAAACAAAGAACTGCA |
| | | CCCGATGGACCTGAAAAACGCGGTTGCGGAAGAACTGATCA |
| | | AAATCCTGGAACCGATCCGTAAACGTCTGTAACTGCAGTTTC |
| | | AAACGCTAAATTGCCTGATGCGCTACGCTTATCAGGCCTACA |
| | | TGATCTCTGCAATATATTGAGTTTGCGTGCTTTTGTAGGCCGG |
| | | ATAAGGCGTTCACGCCGCATCCGGCAAGAAACAGCAAACAA |
| | | TCCAAAACGCCGCGTTCAGCGGCGTTTTTTCTGCTTTTCTTCG |
| | | CGAATTAATTCCGCTTCGCAACATGTGAGCACCGGTTTATTG |
| | | ACTACCGGAAGCAGTGTGACCGTGTGCTTCTCAAATGCCTGA |
| | | GGCCAGTTTGCTCAGGCTCTCCCCGTGGAGGTAATAATTGAC |
| | | GATATGATCAGTGCACGGCTAACTAAGCGGCCTGCTGACTTT |
| | | CTCGCCGATCAAAAGGCATTTTGCTATTAAGGGATTGACGAG |
| | | GGCGTATCTGCGCAGTAAGATGCGCCCCGCATTCCGGCGGTA |
| | | GTTCAGCAGGGCAGAACGGCGGACTCTAAATCCGCATGGCA |
| | | GGGGTTCAAATCCCCTCCGCCGGACCAAATTCGAAAAGCCTG |
| | | CTCAACGAGCAGGCTTTTTTGCATGCTCGAGCAGCTCAGGGT |
| | | CGTTTCAAACGCTAAATTGCCTGATGCGCTACGCTTATCAGG |
| | | CCTACATGATCTCTGCAATATATTGAGTTTGCGTGCTTTTGTA |
| | | GGCCGGATAAGGCGTTCACGCCGCATCCGGCAAGAAACAGC |
| | | AAACAATCCAAAACGCCGCGTTCAGCGGCGTTTTTTCTGCTTT |
| | | TCTTCGCGAATTAATTCCGCTTCGCACATGTGAGCAAAAGGC |
| | | CAGCAAAAGGCCAGGAACCGCTCGAGCGTTTTATCTGTTGTT |
| | | TGTCGGTGAACGCTCTCCTGAGTAGGACAAATCCGCCGGGAG |
| | | CTGTCCCTCCTGTTCAGCTACTGACGGGGTGGTGCGTAACGG |
| | | CAAAAGCACCGCCGGACATCAGCGCTAGCGGAGTGTATACT |
| | | GGCTTACTATGTTGGCACTGATGAGGGTGTCAGTGAAGTGCT |
| | | TCATGTGGCAGGAGAAAAAAGGCTGCACCGGTGCGTCAGCA |
| | | GAATATGTGATACAGGATATATTCCGCTTCCTCGCTCACTGA |
| | | CTCGCTACGCTCGGTCGTTCGACTGCGGCGAGCGGAAATGGC |
| | | TTACGAACGGGGCGGAGATTTCCTGGAAGATGCCAGGAAGA |
| | | TACTTAACAGGGAAGTGAGAGGGCCGCGGCAAAGCCGTTTTT |
| | | CCATAGGCTCCGCCCCCCTGACAAGCATCACGAAATCTGACG |
| | | CTCAAATCAGTGGTGGCGAAACCCGACAGGACTATAAAGAT |
| | | ACCAGGCGTTTCCCCCTGGCGGCTCCCTCGTGCGCTCTCCTGT |
| | | TCCTGCCTTTCGGTTTACCGGTGTCATTCCGCTGTTATGGCCG |
| | | CGTTTGTCTCATTCCACGCCTGACACTCAGTTCCGGGTAGGCA |
| | | GTTCGCTCCAAGCTGGACTGTATGCACGAACCCCCCGTTCAG |
| | | TCCGACCGCTGCGCCTTATCCGGTAACTATCGTCTTGAGTCCA |
| | | ACCCGGAAAGACATGCAAAAGCACCACTGGCAGCAGCCACT |
| | | GGTAATTGATTTAGAGGAGTTAGTCTTGAAGTCAT |
| Pep-MFPVFV | 19 | CTAGCCCCGCGAAATTAATACGACTCACTATAGGGTCTAGAA |
| | | ATAATTTTGTTTAACTTTAAGAAGGAGATATACATATGTTCCC |
| | | CGTCTTCGTCTAATGAGGATCCCGGGAATTCTCGAGTAAGGT |
| | | TAACCTGCAGGAGG |
| Pep-M*PVFV | 20 | CTAGCCCCGCGAAATTAATACGACTCACTATAGGGTCTAGAA |
| | | ATAATTTTGTTTAACTTTAAGAAGGAGATATACATATGTAGC |
| | | CCGTCTTCGTCTAATGAGGATCCCGGGAATTCTCGAGTAAGG |
| | | TTAACCTGCAGGAGG |
| Pep-PFPV*V | 21 | CTAGCCCCGCGAAATTAATACGACTCACTATAGGGTCTAGAA |
| | | ATAATTTTGTTTAACTTTAAGAAGGAGATATACATATGTTCCC |
| | | CGTCTAGGTCTAATGAGGATCCCGGGAATTCTCGAGTAAGGT |
| | | TAACCTGCAGGAGG |
| VHH72 wt ORF | 22 | CAAGTGCAGTTACAAGAGTCGGGTGGTGGACTGGTCCAGGCC |
| | | GGTGGCTCTTTGCGCTTAAGTTGTGCCGCTTCTGGCCGTACTT |
| | | TTTCAGAATACGCAATGGGATGGTTCCGTCAAGCTCCGGGGA |
| | | AAGAACGTGAGTTTGTGGCGACTATTTCCTGGTCTGGGGGGT |
| | | CTACCTACTACACAGACAGCGTAAAAGGCCGTTTCACAATCT |
| | | CGCGCGATAACGCGAAGAATACCGTGTATCTGCAAATGAATT |
| | | CATTGAAACCCGACGACACCGCAGTATATTATTGTGCAGCGG |
| | | CGGGGTTGGGTACGGTCGTTTCTGAGTGGGATTACGATTACG |
| | | ATTACTGGGGCCAGGGTACTCAAGTCACCGTAAGCTCAGGAT |
| | | CA |
| VHH72 | 23 | CAAGTGCAGTTACAAGAGTCGGGTGGTGGACTGGTCCAGGCC |
| W108UAG ORF | | GGTGGCTCTTTGCGCTTAAGTTGTGCCGCTTCTGGCCGTACTT |
| | | TTTCAGAATACGCAATGGGATGGTTCCGTCAAGCTCCGGGGA |
| | | AAGAACGTGAGTTTGTGGCGACTATTTCCTGGTCTGGGGGGT |
| | | CTACCTACTACACAGACAGCGTAAAAGGCCGTTTCACAATCT |
| | | CGCGCGATAACGCGAAGAATACCGTGTATCTGCAAATGAATT |
| | | CATTGAAACCCGACGACACCGCAGTATATTATTGTGCAGCGG |
| | | CGGGGTTGGGTACGGTCGTTTCTGAGTAGGATTACGATTACG |
| | | ATTACTGGGGCCAGGGTACTCAAGTCACCGTAAGCTCAGGAT |
| | | CA |
| Wuhan-NS | 24 | CAAGTGCAGTTACAAGAGTCGGGTGGTGGACTGGTCCAGGCC |
| (VHH72 NoK | | GGTGGCTCTTTGCGCTTAAGTTGTGCCGCTTCTGGCCGTACTT |
| V104K) | | TTTCAGAATACGCAATGGGATGGTTCCGTCAAGCTCCGGGGC |
| | | GTGAACGTGAGTTTGTGGCGACTATTTCCTGGTCTGGGGGGT |
| | | CTACCTACTACACAGACAGCGTACGCGGCCGTTTCACAATCT |
| | | CGCGCGATAACGCGCGTAATACCGTGTATCTGCAAATGAATT |
| | | CATTGCGTCCCGACGACACCGCAGTATATTATTGTGCAGCGG |
| | | CGGGGTTGGGTACGAAAGTTTCTGAGTGGGATTACGATTACG |
| | | ATTACTGGGGCCAGGGTACTCAAGTCACCGTAAGCTCAGGAT |
| | | CTGGTGGTAGTGGAGGAGGTTCTGGTGGTGGTTCTGGT |
| Pep-MFPVFV | 25 | CTAGCCCCGCGAAATTAATACGACTCACTATAGGGTCTAGAA |
| | | ATAATTTTGTTTAACTTTAAGAAGGAGATATACATATGTTCCC |
| | | CGTCTTCGTCTAATGAGGATCCCGGGAATTCTCGAGTAAGGT |
| | | TAACCTGCAGGAGG |
| Pep-M*PVFV | 26 | CTAGCCCCGCGAAATTAATACGACTCACTATAGGGTCTAGAA |
| | | ATAATTTTGTTTAACTTTAAGAAGGAGATATACATATGTAGC |
| | | CCGTCTTCGTCTAATGAGGATCCCGGGAATTCTCGAGTAAGG |
| | | TTAACCTGCAGGAGG |
| Pep-PFPV*V | 27 | CTAGCCCCGCGAAATTAATACGACTCACTATAGGGTCTAGAA |
| | | ATAATTTTGTTTAACTTTAAGAAGGAGATATACATATGTTCCC |
| | | CGTCTAGGTCTAATGAGGATCCCGGGAATTCTCGAGTAAGGT |
| | | TAACCTGCAGGAGG |
| Omicron-NS-1 | 28 | CAAGTGCAGTTACAAGAGTCGGGTGGTGGACTGGTCCAGGCC |
| (V104UAG) | | GGTGGCTCTTTGCGCTTAAGTTGTGCCGCTTCTGGCCGTACTT |
| | | TTTCAGAATACGCAATGGGATGGTTCCGTCAAGCTCCGGGGA |
| | | AAGAACGTGAGTTTGTGGCGACTATTGGTCCGTCTGGCGGCG |
| | | TTACCGGTTACACAGACAGCGTAAAAGGCCGTTTCACAATCT |
| | | CGCGCGATAACGCGAAGAATACCGTGTATCTGCAAATGAATT |
| | | CATTGAAACCCGACGACACCGCAGTATATTATTGTGCAGCGG |
| | | CGGGGTTGGGTACGTAGGTTTCTGAGTGGGATTACGATTACG |
| | | ATTACTGGGGCCAGGGTACTCAAGTCACCGTAAGCTCAGGAT |
| | | CTGGTGGTGGAAGCGGCGGTTCTGGTGGTGGTTCTGGT |
| Omicron-NS-2 | 29 | CAAGTGCAGTTACAAGAGTCGGGTGGTGGACTGGTCCAGGCC |
| (V104UAG) | | GGTGGCTCTTTGCGCTTAAGTTGTGCCGCTTCTGGCCGTACTT |
| | | TTTCAGAATACGCAATGGGATGGTTCCGTCAAGCTCCGGGGA |
| | | AAGAACGTGAGTTTGTGGCGACTATTGGTCCGTCTGGCGGCA |
| | | TTACCGGTTACACAGACAGCGTAAAAGGCCGTTTCACAATCT |
| | | CGCGCGATAACGCGAAGAATACCGTGTATCTGCAAATGAATT |
| | | CATTGAAACCCGACGACACCGCAGTATATTATTGTGCAGCGG |
| | | CGGGGTTGGGTACGTAGGTTTCTGAGTGGGATTACGATTACG |
| | | ATTACTGGGGCCAGGGTACTCAAGTCACCGTAAGCTCAGGAT |
| | | CTGGTGGTGGAAGCGGCGGTTCTGGTGGTGGTTCTGGT |
| Omicron-NS-3 | 30 | CAAGTGCAGTTACAAGAGTCGGGTGGTGGACTGGTCCAGGCC |
| (V104UAG) | | GGTGGCTCTTTGCGCTTAAGTTGTGCCGCTTCTGGCCGTACTT |
| | | TTTCAGAATACGCAATGGGATGGTTCCGTCAAGCTCCGGGGC |
| | | GTGAACGTGAGTTTGTGGCGACTATTCTTCGGTCTGGCGGCA |
| | | GTACCTTTTACACAGACAGCGTACGCGGCCGTTTCACAATCT |
| | | CGCGCGATAACGCGCGTAATACCGTGTATCTGCAAATGAATT |
| | | CATTGCGTCCCGACGACACCGCAGTATATTATTGTGCAGCGG |
| | | CGGGGTTGGGTACGTAGGTTTCTGAGTGGGATTACGATTACG |
| | | ATTACTGGGGCCGCGGTACTCAAGTCACCGTAAGCTCAGGAT |
| | | CTGGTGGTGGAAGCGGCGGTTCTGGTGGTGGTTCTGGT |
| Omicron-NS-1 | 31 | CAAGTGCAGTTACAAGAGTCGGGTGGTGGACTGGTCCAGGCC |
| (NoK V104K) | | GGTGGCTCTTTGCGCTTAAGTTGTGCCGCTTCTGGCCGTACTT |
| | | TTTCAGAATACGCAATGGGATGGTTCCGTCAAGCTCCGGGGC |
| | | GTGAACGTGAGTTTGTGGCGACTATTGGTCCGTCTGGCGGCG |
| | | TTACCGGTTACACAGACAGCGTACGCGGCCGTTTCACAATCT |
| | | CGCGCGATAACGCGCGTAATACCGTGTATCTGCAAATGAATT |
| | | CATTGCGTCCCGACGACACCGCAGTATATTATTGTGCAGCGG |
| | | CGGGGTTGGGTACGAAAGTTTCTGAGTGGGATTACGATTACG |
| | | ATTACTGGGGCCAGGGTACTCAAGTCACCGTAAGCTCAGGAT |
| | | CTGGTGGTGGAAGCGGCGGTTCTGGTGGTGGTTCTGGT |
| Omicron-NS-2 | 32 | CAAGTGCAGTTACAAGAGTCGGGTGGTGGACTGGTCCAGGCC |
| (NoK V104K) | | GGTGGCTCTTTGCGCTTAAGTTGTGCCGCTTCTGGCCGTACTT |
| | | TTTCAGAATACGCAATGGGATGGTTCCGTCAAGCTCCGGGGC |
| | | GTGAACGTGAGTTTGTGGCGACTATTGGTCCGTCTGGCGGCA |
| | | TTACCGGTTACACAGACAGCGTACGCGGCCGTTTCACAATCT |
| | | CGCGCGATAACGCGCGTAATACCGTGTATCTGCAAATGAATT |
| | | CATTGCGTCCCGACGACACCGCAGTATATTATTGTGCAGCGG |
| | | CGGGGTTGGGTACGAAAGTTTCTGAGTGGGATTACGATTACG |
| | | ATTACTGGGGCCAGGGTACTCAAGTCACCGTAAGCTCAGGAT |
| | | CTGGTGGTGGAAGCGGCGGTTCTGGTGGTGGTTCTGGT |
| Omicron-NS-3 | 33 | CAAGTGCAGTTACAAGAGTCGGGTGGTGGACTGGTCCAGGCC |
| (NoK V104K) | | GGTGGCTCTTTGCGCTTAAGTTGTGCCGCTTCTGGCCGTACTT |
| | | TTTCAGAATACGCAATGGGATGGTTCCGTCAAGCTCCGGGGA |
| | | AAGAACGTGAGTTTGTGGCGACTATTCTTCGGTCTGGCGGCA |
| | | GTACCTTTTACACAGACAGCGTAAAAGGCCGTTTCACAATCT |
| | | CGCGCGATAACGCGAAGAATACCGTGTATCTGCAAATGAATT |
| | | CATTGAAACCCGACGACACCGCAGTATATTATTGTGCAGCGG |
| | | CGGGGTTGGGTACGAAAGTTTCTGAGTGGGATTACGATTACG |
| | | ATTACTGGGGCAAGGGTACTCAAGTCACCGTAAGCTCAGGAT |
| | | CTGGTGGTGGAAGCGGCGGTTCTGGTGGTGGTTCTGGT |
| H11-H4 wt ORF | 34 | CAAGTCCAATTAGTCGAGAGTGGCGGTGGTCTTATGCAAGCC |
| | | GGAGGGAGTTTACGCCTTTCCTGCGCGGTATCGGGGCGTACT |
| | | TTCAGTACTGCGGCGATGGGCTGGTTCCGTCAAGCGCCCGGA |
| | | AAAGAGCGCGAGTTCGTCGCTGCTATTCGCTGGTCGGGGGGC |
| | | TCAGCATATTACGCTGATAGCGTAAAGGGACGCTTCACCATT |
| | | TCGCGTGACAAGGCTAAAAATACTGTATATCTGCAGATGAAC |
| | | TCACTGAAATACGAGGACACAGCTGTCTACTATTGTGCCCAG |
| | | ACACATTACGTGTCGTACTTGTTAAGTGATTATGCAACCTGG |
| | | CCCTACGACTACTGGGGCCAAGGAACTCAGGTAACGGTTTCA |
| | | TCGGGATCTGGTGGTAGTGGAGGAGGTTCTGGTGGTGGTTCT |
| | | GGT |
| sdAb-B6 wt ORF | 35 | GAGGTGCAACTGCAGGCTAGCGGGGGTGGATTAGTCCGCCCT |
| | | GGTGGCTCGCTTCGCTTGAGCTGCGCTGCAAGCGGTTTTACCT |
| | | TTTCATCATACGCCATGATGTGGGTCCGTCAGGCACCGGGTA |
| | | AGGGGCTTGAATGGGTATCTGCGATCAACGGAGGCGGAGGT |
| | | TCGACGAGCTATGCAGATAGTGTAAAAGGACGCTTCACCATT |
| | | TCACGTGATAATGCAAAGAATACATTGTACCTTCAGATGAAC |
| | | TCCCTGAAACCGGAGGACACAGCCGTCTATTACTGCGCTAAG |
| | | TACCAGGCTGCAGTACACCAAGAGAAGGAAGACTACTGGGG |
| | | TCAAGGCACGCAGGTAACCGTATCGTCTGGATCTGGTGGTAG |
| | | TGGAGGAGGTTCTGGTGGTGGTTCTGGT |
| LCB1 wt ORF | 36 | ATGGATAAAGAATGGATTCTTCAAAAAATCTACGAGATCATG |
| | | CGTTTGTTAGACGAGCTGGGCCACGCGGAAGCGAGTATGCGC |
| | | GTTTCAGATTTAATCTACGAGTTTATGAAGAAAGGTGATGAA |
| | | CGTCTGTTGGAGGAAGCGGAACGTCTGCTTGAGGAAGTAGA |
| | | GCGC |
| LCB3 wt ORF | 37 | ATGAACGATGATGAGCTGCATATGTTAATGACGGATCTTGTG |
| | | TATGAGGCACTGCATTTTGCTAAGGATGAAGAGATCAAGAAG |
| | | CGCGTATTCCAACTTTTTGAATTGGCCGACAAAGCCTACAAA |
| | | AACAATGACCGTCAAAAACTTGAAAAGGTGGTTGAGGAATT |
| | | GAAGGAGTTATTAGAACGCTTATTGTCA |
| NbALFA wt ORF | 38 | CAATTACAGGAGAGCGGCGGAGGATTGGTACAGCCCGGAGG |
| | | ATCGCTGCGCTTAAGTTGTACTGCAAGTGGAGTTACGATTTC |
| | | GGCCCTGAACGCTATGGCAATGGGCTGGTATCGCCAAGCACC |
| | | GGGTGAGCGCCGTGTGATGGTAGCTGCCGTGTCCGAACGTGG |
| | | CAATGCTATGTACCGTGAATCGGTCCAAGGTCGTTTTACAGT |
| | | GACACGCGATTTCACAAATAAAATGGTGTCATTACAGATGGA |
| | | CAATCTTAAACCCGAGGATACCGCAGTCTACTATTGCCACGT |
| | | CCTTGAAGATCGCGTAGACTCCTTTCACGATTATTGGGGCCA |
| | | GGGAACCCAGGTTACAGTCAGCTCTGGATCTGGTGGTAGTGG |
| | | AGGAGGTTCTGGTGGTGGTTCTGGT |
| EgA1 wt ORF | 39 | CAGGTACAGCTGCAAGAATCTGGAGGAGGCCTGGTACAACC |
| | | CGGTGGGAGCCTGCGGTTATCTTGCGCAGCTTCTGGTCGTAC |
| | | CTTTTCAAGTTATGCGATGGGGTGGTTCCGTCAGGCCCCGGG |
| | | AAAACAGAGAGAATTCGTGGCCGCCATACGTTGGTCGGGCG |
| | | GCTACACATACTATACGGATTCAGTGAAGGGCCGGTTCACCA |
| | | TTTCCCGTGATAATGCGAAGACCACCGTGTACCTGCAGATGA |
| | | ATTCCCTTAAGCCTGAAGATACCGCGGTGTACTACTGCGCTG |
| | | CCACATACCTGAGCAGCGACTATAGTCGGTACGCTCTCCCAC |
| | | AACGTCCACTCGATTACGATTATTGGGGACAGGGAACACAGG |
| | | TCACAGTAAGCTCAGGTAGTGGCGGTTCAGGCGGGGGTTCTG |
| | | GTGGAGGCTCCGGG |
| NbCor wt ORF | 40 | CAGGTGCAATTACAGGAGTCTGGAGGGGGCTCGGTCCAGGC |
| | | GGGGGGCTCGTTACGCCTTAGCTGTGTAGTTAGCGGTAACAC |
| | | AGGATCTACCGGCTATTGGGCATGGTTTCGTCAAGGCCCAGG |
| | | AACCGAACGCGAGGGGGTCGCTGCTACTTATACAGCAGGGTC |
| | | AGGCACGTCAATGACTTACTATGCGGACTCGGTGAAAGGACG |
| | | CTTCACTATTAGCCAGGACAATGCAAAAAAAACGTTGTATTT |
| | | GCAGATGAACTCTCTGAAACCTGAGGATACCGGAATGTACCG |
| | | TTGCGCTTCAACTCGCTTCGCGGGCCGTTGGTACCGCGACTCT |
| | | GAATATCGCGCTTGGGGTCAGGGAACCCAGGTTACGGTATCG |
| | | AGTGGATCTGGTGGTAGTGGAGGAGGTTCTGGTGGTGGTTCT |
| | | GGT |
| pTB107 | 41 | GACGAAAGGGCCTCGTGATACGCCTATTTTTATAGGTTAATG |
| (pLOW_ALFA- | | TCATGATAATAATGGTTTCTTAGACGTCAGGTGGCACTTTTCG |
| spa-LPXTG) | | GGGAAATGTGCGCGGAACCCCTATTTGTTTATTTTTCTAAATA |
| | | CATTCAAATATGTATCCGCTCATGAGACAATAACCCTGATAA |
| | | ATGCTTCAATAATATTGAAAAAGGAAGAGTATGAGTATTCAA |
| | | CATTTCCGTGTCGCCCTTATTCCCTTTTTTGCGGCATTTTGCCT |
| | | TCCTGTTTTTGCTCACCCAGAAACGCTGGTGAAAGTAAAAGA |
| | | TGCTGAAGATCAGTTGGGTGCACGAGTGGGTTACATCGAACT |
| | | GGATCTCAACAGCGGTAAGATCCTTGAGAGTTTTCGCCCCGA |
| | | AGAACGTTTTCCAATGATGAGCACTTTTAAAGTTCTGCTATGT |
| | | GGCGCGGTATTATCCCGTATTGACGCCGGGCAAGAGCAACTC |
| | | GGTCGCCGCATACACTATTCTCAGAATGACTTGGTTGAGTAC |
| | | TCACCAGTCACAGAAAAGCATCTTACGGATGGCATGACAGTA |
| | | AGAGAATTATGCAGTGCTGCCATAACCATGAGTGATAACACT |
| | | GCGGCCAACTTACTTCTGACAACGATCGGAGGACCGAAGGA |
| | | GCTAACCGCTTTTTTGCACAACATGGGGGATCATGTAACTCG |
| | | CCTTGATCGTTGGGAACCGGAGCTGAATGAAGCCATACCAAA |
| | | CGACGAGCGTGACACCACGATGCCTGTAGCAATGGCAACAA |
| | | CGTTGCGCAAACTATTAACTGGCGAACTACTTACTCTAGCTTC |
| | | CCGGCAACAATTAATAGACTGGATGGAGGCGGATAAAGTTG |
| | | CAGGACCACTTCTGCGCTCGGCCCTTCCGGCTGGCTGGTTTAT |
| | | TGCTGATAAATCTGGAGCCGGTGAGCGTGGGTCTCGCGGTAT |
| | | CATTGCAGCACTGGGGCCAGATGGTAAGCCCTCCCGTATCGT |
| | | AGTTATCTACACGACGGGGAGTCAGGCAACTATGGATGAAC |
| | | GAAATAGACAGATCGCTGAGATAGGTGCCTCACTGATTAAGC |
| | | ATTGGTAACTGTCAGACCAAGTTTACTCATATATACTTTAGAT |
| | | TGATTTAAAACTTCATTTTTAATTTAAAAGGATCTAGGTGAA |
| | | GATCCTTTTTGATAATCTCATGACCAAAATCCCTTAACGTGAG |
| | | TTTTCGTTCCACTGAGCGTCAGACCCCGTAGAAAAGATCAAA |
| | | GGATCTTCTTGAGATCCTTTTTTTCTGCGCGTAATCTGCTGCT |
| | | TGCAAACAAAAAAACCACCGCTACCAGCGGTGGTTTGTTTGC |
| | | CGGATCAAGAGCTACCAACTCTTTTTCCGAAGGTAACTGGCT |
| | | TCAGCAGAGCGCAGATACCAAATACTGTCCTTCTAGTGTAGC |
| | | CGTAGTTAGGCCACCACTTCAAGAACTCTGTAGCACCGCCTA |
| | | CATACCTCGCTCTGCTAATCCTGTTACCAGTGGCTGCTGCCAG |
| | | TGGCGATAAGTCGTGTCTTACCGGGTTGGACTCAAGACGATA |
| | | GTTACCGGATAAGGCGCAGCGGTCGGGCTGAACGGGGGGTT |
| | | CGTGCACACAGCCCAGCTTGGAGCGAACGACCTACACCGAA |
| | | CTGAGATACCTACAGCGTGAGCTATGAGAAAGCGCCACGCTT |
| | | CCCGAAGGGAGAAAGGCGGACAGGTATCCGGTAAGCGGCAG |
| | | GGTCGGAACAGGAGAGCGCACGAGGGAGCTTCCAGGGGGAA |
| | | ACGCCTGGTATCTTTATAGTCCTGTCGGGTTTCGCCACCTCTG |
| | | ACTTGAGCGTCGATTTTTGTGATGCTCGTCAGGGGGGCGGAG |
| | | CCTATGGAAAAACGCCAGCAACGCGGCCTTTTTACGGTTCCT |
| | | GGCCTTTTGCTGGCCTTTTGCTCACATGTTCTTTCCTGCGTTAT |
| | | CCCCTGATTCTGTGGATAACCGTATTACCGCCTTTGAGTGAGC |
| | | TGATACCGCTCGCCGCAGCCGAACGACCGAGCGCAGCGAGT |
| | | CAGTGAGCGAGGAAGCGGAAGAGCGCCCAATACGCAAACCG |
| | | CCTCTCCCCGCGCGTTGGCCGATTCATTAATGCAGCTGGCAC |
| | | GACAGGTTTCCCGACTGGAAAGCGGGCAGTGAGCGCAACGC |
| | | AATTAATGTGAGTTAGCTCACTCATTAGGCACCCCAGGCTTT |
| | | ACACTTTATGCTTCCGGCTCGTATGTTGTGTGGAATTGTGAGC |
| | | GGATAACAATTTCACACAGGAAACAGCTATGACCATGATTAC |
| | | GCCAAGCTAAGCTTTAATTACTTTAACCATTGCTACCTTCGTT |
| | | GAAGGTGCCTGATCTGTAATTACCTTTTGAGGTTTACCAAATT |
| | | GTTTAATGAGACGTTTGATAAACGCATATGCTGAATGATTAT |
| | | CTCGTTGCTTACGCAACCAAATATCTAATGTATGTCCCTCTGC |
| | | ATCAATGGCACGATATAAATAGCTCCATTTTCCTTTTATTTTG |
| | | ATGTACGTCTCATCAATACGCCATTTGTAATAAGCTTTTTTAT |
| | | GCTTTTTCTTCCAAATTTGATATAAAATTGGGGCATATTCTTG |
| | | AACCCAACGGTAGACCGTTGAATGATGAACGTTTACACCACG |
| | | TCCCCTTAATATTTCAGATATATCACGATAACTCAATGCATAT |
| | | CTTAGATAGTAGCCAACGGCTACAGTGATAACATCCTTGTTA |
| | | AATTGTTTATATCTGAAATAGTTCATACAGAAGACTCCTTTTT |
| | | GTTAAAATTATACTATAAATTCAACTTTGCAACAGAACCGTA |
| | | AAGATTATACACCTTTTTAGGTGTATAATCTCTTTTTTTTGAA |
| | | AATTTAACTTCAAAAACTAATTAAATAGAGATAATGGATTGT |
| | | TTTCCATATTTTTTCTTCTCATAATAGTTGAGTGCATTTCTTCA |
| | | AGTTCAGCAATGATACTTCTCATTAAATAACCTTCCATATTAG |
| | | CTACTTTTTCTTGTTTTTTAACAAGATAACCTTTAAATCGTTTT |
| | | AAAACCAATAGTAATTCTTCATCTATATCTTCTAACATATAGA |
| | | AAGTATCGTATTTGTTATTAAATGATTTTTTAGCTTTTAAAAT |
| | | AACAGATTTAATACTTTTAACTTCTTCATAGCTGAAATTATTA |
| | | ATATAACTTTTAATTAATTCGGGGAGTTCTTGAAGTTCTATAT |
| | | ATTTAAGAGATTCTTTATCATGAATATTTGAAAAGTGATTTGA |
| | | ATGATTTGAATGTTGATTTGTATCATTCATATTATTCATATCA |
| | | TTACTTTCAGTATCAATAAAATCAGTATCAATAAAATCAGTA |
| | | TCATTTGTGTGTCCTTTTGACATTTCTAGACGTGTCCTTTTGAC |
| | | ATTTCTAGACGTGTCCTTTTGACATTTCTGGACGTGTCCTTTT |
| | | GACACTTCCTTGTCTTGTAAGGCCTCAACTTCATTTTCAGCCT |
| | | TATCTATTTCATAAATATCATTTTTAGTTATGGCTGGTTTTAA |
| | | TAAATAAAGTAGATTTGGTTTGTTTAAACCCTGCCTTTTTTGG |
| | | ATTAGTAAATCTACATTTTCTAATTCTTTTTTAATTTTAGTGAT |
| | | TTTTTTGTTCCCACAATTTAATATCACTTCTAAATCAGCAACT |
| | | GTATAAATGAAATATATGTTACCTTCTGTATCTATCCAGTTAT |
| | | TTTTAATAGATAATTGTAAACGATCTCTCAATATTGCGTAAGC |
| | | AATTTTAGCGTCATTCGATAAATCTTTATAATTAGGATTAGTA |
| | | AAAAATACTTTAGGTAATTGGTAAAAGCGTTCTTTATAATTTT |
| | | CTTCTACTGTAAAAAATTGTTTAGACATGATAAAAACTCCTTT |
| | | AAATGTATATTTAAGGAGTTTAAGTAATATCGTTATTTACAA |
| | | GTGATGAATAAGGTTATATAATGTTATCCGTTATATATAACTT |
| | | AAAGACTTGTTGCTTTTATCTTGTAATGTATCGAGGTTGTATA |
| | | TATAATAAAGATACAAACAAATTGTAAAAGTAAATAACACA |
| | | TATTATTTAAACTCTTTAGACATCTAACAACCGCCAAGCGTTA |
| | | GGTGTCTTTTTCTATGTTCAATAGTAACACTATTTTAAAAAAA |
| | | TTAAAAATTTTTTGTTGCCTCACAGGTCAATTATATGCTTGTA |
| | | ATGAAATGTATTTGATAACAAATCTCTAATATAATTAATATA |
| | | TCTAAAATTATGAGGCCGATAAATCATGAATGAATACCGTAT |
| | | ACAAAAACTATATGGATACAGATCCCATATAATTTTTGAAGC |
| | | TTGCGTTGCGCTCACTGCCCGCTTTCCAGTCGGGAAACCTGTC |
| | | GTGCCAGCTGCATTAATGAATCGGCCAACGCGCGGGGAGAG |
| | | GCGGTTTGCGTATTGGGCGCCAGGGTGGTTTTTCTTTTCACCA |
| | | GTGAGACGGGCAACAGCTGATTGCCCTTCACCGCCTGGCCCT |
| | | GAGAGAGTTGCAGCAAGCGGTCCACGCTGGTTTGCCCCAGCA |
| | | GGCGAAAATCCTGTTTGATGGTGGTTGACGGCGGGATATAAC |
| | | ATGAGCTGTCTTCGGTATCGTCGTATCCCACTACCGAGATATC |
| | | CGCACCAACGCGCAGCCCGGACTCGGTAATGGCGCGCATTGC |
| | | GCCCAGCGCCATCTGATCGTTGGCAACCAGCATCGCAGTGGG |
| | | AACGATGCCCTCATTCAGCATTTGCATGGTTTGTTGAAAACC |
| | | GGACATGGCACTCCAGTCGCCTTCCCGTTCCGCTATCGGCTG |
| | | AATTTGATTGCGAGTGAGATATTTATGCCAGCCAGCCAGACG |
| | | CAGACGCGCCGAGACAGAACTTAATGGGCCCGCTAACAGCG |
| | | CGATTTGCTGGTGACCCAATGCGACCAGATGCTCCACGCCCA |
| | | GTCGCGTACCGTCTTCATGGGAGAAAATAATACTGTTGATGG |
| | | GTGTCTGGTCAGAGACATCAAGAAATAACGCCGGAACATTA |
| | | GTGCAGGCAGCTTCCACAGCAATGGCATCCTGGTCATCCAGC |
| | | GGATAGTTAATGATCAGCCCACTGACGCGTTGCGCGAGAAGA |
| | | TTGTGCACCGCCGCTTTACAGGCTTCGACGCCGCTTCGTTCTA |
| | | CCATCGACACCACCACGCTGGCACCCAGTTGATCGGCGCGAG |
| | | ATTTAATCGCCGCGACAATTTGCGACGGCGCGTGCAGGGCCA |
| | | GACTGGAGGTGGCAACGCCAATCAGCAACGACTGTTTGCCCG |
| | | CCAGTTGTTGTGCCACGCGGTTGGGAATGTAATTCAGCTCCG |
| | | CCATCGCCGCTTCCACTTTTTCCCGCGTTTTCGCAGAAACGTG |
| | | GCTGGCCTGGTTCACCACGCGGGAAACGGTCTGATAAGAGAC |
| | | ACCGGCATACTCTGCGACATCGTATAACGTTACTGGTTTCATC |
| | | AAATCGTCTCCCTCCGTTTGAATATTTGATTGATCGTAACCAG |
| | | ATCAAGCACTCTTTCCACTATCCCTACAGTGTTATGGCTTGAA |
| | | CAATCACGAAACAATAATTGGTACGTACGATCTTTCAGCCGA |
| | | CTCAAACATCAAATCTTACAAATGTAGTCTTTGAAAGTATTA |
| | | CATATGTAAGATTTAAATGCAACCGTTTTTTCGGAAGGAAAT |
| | | GATGACCTCGTTTCCACCGAATTAGCTTGAAATAGTACATAA |
| | | TGGATTTCCTTACGCGAAATACGGGCAGACATGGCCTGCCCG |
| | | GTTATTATTATTTTTGACACCGCATGCTGCGGTACCGTTAAGG |
| | | GATGCAGTTTATGCATCCCTTAACTTACTTATTAAATAATTTA |
| | | TAGCTATTGAAAAGAGATAAGAATTGTTCAAAGCTAATATTG |
| | | TTTAAATCGTCAATTCCTGCATGTTTTAAGGAATTGTTAAATT |
| | | GATTTTTTGTAAATATTTTCTTGTATTCTTTGTTAACCCATTTC |
| | | ATAACGAAATAATTATACTTTTGTTTATCTTTGTGTGATATTC |
| | | TTGATTTTTTTCTACTTAATCTGATAAGTGAGCTATTCACTTT |
| | | AGGTTTAGGATGAAAATATTCTCTTGGAACCATACTTAATAT |
| | | AGAAATATCAACTTCTGCCATTAAAAGTAATGCCAATGAGCG |
| | | TTTTGTATTTAATAATCTTTTAGCAAACCCGTATTCCACGATT |
| | | AAATAAATCTCATTAGCTATACTATCAAAAACAATTTTGCGT |
| | | ATTATATCCGTACTTATGTTATAAGGTATATTACCATATATTT |
| | | TATAGGATTGGTTTTTAGGAAATTTAAACTGCAATATATCCTT |
| | | GTTTAAAACTTGGAAATTATCGTGATCAACAAGTTTATTTTCT |
| | | GTAGTTTTGCATAATTTATGGTCTATTTCAATGGCAGTTACGA |
| | | AATTACACCTCTTTACTAATTCAAGGGTAAAATGGCCTTTTCC |
| | | TGAGCCGATTTCAAAGATATTATCATGTTCATTTAATCTTATA |
| | | TTTGTCATTATTTTATCTATATTATGTTTTGAAGTAATAAAGT |
| | | TTTGACTGTGTTTTATATTTTTCTCGTTCATTATAACCCTCTTT |
| | | AATTTGGTTATATGAATTTTGCTTATTAACGATTCATTATAAC |
| | | CACTTATTTTTTGTTTGGTTGATAATGAACTGTGCTGATTACA |
| | | AAAATACTAAAAATGCCCATATTTTTTCCTCCTTATAAAATTA |
| | | GTATAATTATAGCACGAGCTCTGATAAATATGAACATGATGA |
| | | GTGATCGTTAAATTTATACTGCAATCGGATGCGATTATTGAA |
| | | TAAAAGATATGAGAGATTTATCTAATTTCTTTTTTCTTGTAAA |
| | | AAAAGAAAGTTCTTAAAGGTTTTATAGTTTTGGTCGTAGAGC |
| | | ACACGGTTTAACGACTTAATTACGAAGTAAATAAGTCTAGTG |
| | | TGTTAGACTTTATGAAATCTATATACGTTTATATATATTTATT |
| | | ATCCGGAGGTGTAGCATGTCTCATTCAATTCCTAGGTGGGCC |
| | | CAATAAAAGCAATCAATGAACCAAGACAGCATCGATCCTCTA |
| | | GAGTCAAATGTGAGCAGTAACAACCTCTGCTAAAATTCCTGA |
| | | AAAATTTTGCAAAAAGTTGTTGACTTTATCTACAAGGTGTGG |
| | | CATAATGTGTGGAATTGTGAGCGCTCACAATTGACCTGCAGG |
| | | CATGCCTGCAGGTCGACACATAAGGAGGAGGTACCTTGAAA |
| | | AAGAAAAACATTTATTCAATTCGTAAACTAGGTGTAGGTATT |
| | | GCATCTGTAACTTTAGGTACATTACTTATATCTGGTGGCGTAA |
| | | CACCTGCTGCAAATGCTGCGCCGAGCCGCTTAGAAGAGGAAT |
| | | TAAGAAGAAGATTAACAGAACCGGCGCAACACGATGAAGCT |
| | | CAACAAAATGCTTTTTATCAAGTCTTAAATATGCCTAACTTAA |
| | | ATGCTGATCAACGCAATGGTTTTATCCAAAGCCTTAAAGATG |
| | | ATCCAAGCCAAAGTGCTAACGTTTTAGGTGAAGCTCAAAAAC |
| | | TTAATGACTCTCAAGCTCCAAAAGCTGATGCGCAACAAAATA |
| | | ACTTCAACAAAGATCAACAAAGCGCCTTCTATGAAATCTTGA |
| | | ACATGCCTAACTTAAACGAAGCGCAACGTAACGGCTTCATTC |
| | | AAAGTCTTAAAGACGACCCAAGCCAAAGCACTAACGTTTTAG |
| | | GTGAAGCTAAAAAATTAAACGAATCTCAAGCACCGAAAGCT |
| | | GATAACAATTTCAACAAAGAACAACAAAATGCTTTCTATGAA |
| | | ATCTTGAATATGCCTAACTTAAACGAAGAACAACGCAATGGT |
| | | TTCATCCAAAGCTTAAAAGATGACCCAAGCCAAAGTGCTAAC |
| | | CTATTGTCAGAAGCTAAAAAGTTAAATGAATCTCAAGCACCG |
| | | AAAGCGGATAACAAATTCAACAAAGAACAACAAAATGCTTT |
| | | CTATGAAATCTTACATTTACCTAACTTAAACGAAGAACAACG |
| | | CAATGGTTTCATCCAAAGCCTAAAAGATGACCCAAGCCAAAG |
| | | CGCTAACCTTTTAGCAGAAGCTAAAAAGCTAAATGATGCTCA |
| | | AGCACCAAAAGCTGACAACAAATTCAACAAAGAACAACAAA |
| | | ATGCTTTCTATGAAATTTTACATTTACCTAACTTAACTGAAGA |
| | | ACAACGTAACGGCTTCATCCAAAGCCTTAAAGACGATCCTTC |
| | | AGTGAGCAAAGAAATTTTAGCAGAAGCTAAAAAGCTAAACG |
| | | ATGCTCAAGCACCAAAAGAGGAAGACAATAACAAGCCTGGC |
| | | AAAGAAGACAATAACAAGCCTGGCAAAGAAGACAACAACAA |
| | | GCCTGGTAAAGAAGACAACAACAAGCCTGGTAAAGAAGACA |
| | | ACAACAAGCCTGGCAAAGAAGACGGCAACAAGCCTGGTAAA |
| | | GAAGACAACAAAAAACCTGGTAAAGAAGATGGCAACAAGCC |
| | | TGGTAAAGAAGACAACAAAAAACCTGGTAAAGAAGACGGCA |
| | | ACAAGCCTGGCAAAGAAGATGGCAACAAACCTGGTAAAGAA |
| | | GATGGTAACGGAGTACATGTCGTTAAACCTGGTGATACAGTA |
| | | AATGACATTGCAAAAGCAAACGGCACTACTGCTGACAAAATT |
| | | GCTGCAGATAACAAATTAGCTGATAAAAACATGATCAAACCT |
| | | GGTCAAGAACTTGTTGTTGATAAGAAGCAACCAGCAAACCAT |
| | | GCAGATGCTAACAAAGCTCAAGCATTACCAGAAACTGGTGA |
| | | AGAAAATCCATTCATCGGTACAACTGTATTTGGTGGATTATC |
| | | ATTAGCCTTAGGTGCAGCGTTATTAGCTGGACGTCGTCGCGA |
| | | ACTATAAGGATCCCCGGGCGAGCTCGAATTCACTGGCCGTCG |
| | | TTTTACAACGTCGTGACTGGGAAAACCCTGGCGTTACCCAAC |
| | | TTAATCGCCTTGCAGCACATCCCCCTTTCGCCAGCTGGCGTAA |
| | | TAGCGAAGAGGCCCGCACCGATCGCCCTTCCCAACAGTTGCG |
| | | CAGCCTGAATGGCGAATGGCGCCTGATGCGGTATTTTCTCCT |
| | | TACGCATCTGTGCGGTATTTCACACCGCATATGGTGCACTCTC |
| | | AGTACAATCTGCTCTGATGCCGCATAGTTAAGCCAGCCCCGA |
| | | CACCCGCCAACACCCGCTGACGCGCCCTGACGGGCTTGTCTG |
| | | CTCCCGGCATCCGCTTACAGACAAGCTGTGACCGTCTCCGGG |
| | | AGCTGCATGTGTCAGAGGTTTTCACCGTCATCACCGAAACGC |
| | | GCGA |

TABLE 4
Excitation/emission wavelengths used to detect fluorescence
of nanosensors modified with the shown probes

| Name | λ_{Ex} | λ_{Em} |
|---|---|---|
| IANBD/NBD-x-NHS/NBD-dodeca-NHS | 485 | 528 |
| | 420 | 528 |
| RhoBITC | 540 | 585 |
| Atto Rho3B-Mal, | 540 | 585 |
| Cy3-Mal | 550 | 580 |
| MDCc, MDCpc, DCc-NHS | 420 | 450 |
| IAEDANS | 420 | 450 |
| IAMG/MGITC | 616 | 650 |
| AO-Mal | 485 | 528 |
| APM-o-NHS | 616 | 650 |
| RhoRed-x-NHS | 550 | 580 |

TABLE 5
DNA/RNA sequences used

| Name | SEQ ID NO. | Sequence |
|---|---|---|
| Pri1 | 42 | GCTCCGGGGAAAGAACGTGAGTTTGTGGCGRSC |
| | | ATTRVCNNCTCTGGGRSCANCACCWACTACACA |
| | | GACAGCGTAAAAGGCCGTTTCACAATCTCGCGC |
| | | GATAACGCGAAGAATACCGTGTATCTGCAAATG |
| Pri2 | 43 | CCCTGGCCCCAGTAATCGTAATCGNNATCGNNC |
| | | TCAGAGNNCTAGNNACCCAACCCCGCCGCTGCA |
| | | CAATAATATACTGCGGTGTCGTCGGGTTTCAAT |
| | | GAATTCATTTGCAGATACACGGTATTCTTCGCGT |
| | | TATCG |
| Pri3 (samples | 44 | GCTCCGGGGAAAGAACGTGAGTTTGTGGCGRSC |
| interface | | ATTRVCTGGTCTGGGRSCANCACCWACTACACA |
| tryptophans) | | GACAGCGTAAAAGGCCGTTTCACAATCTCGCGC |
| | | GATAACGCGAAGAATACCGTGTATCTGCAAATG |
| Pri4 (samples | 45 | CCCTGGCCCCAGTAATCGTAATCGNNATCCCAC |
| interface | | TCAGAGNNCTAGNNACCCAACCCCGCCGCTGCA |
| tryptophans) | | CAATAATATACTGCGGTGTCGTCGGGTTTCAAT |
| | | GAATTCATTTGCAGATACACGGTATTCTTCGCGT |
| | | TATCG |
| Pri5 | 46 | GCTCCGGGGAAAGAACGTGAG |
| Pri6 | 47 | CCCTGGCCCCAGTAATCGTAATCG |
| Pri7 | 48 | CGATTACGATTACTGGGGCCAGG |
| Pri8 | 49 | CTCACGTTCTTTCCCCGGAGC |
| Pri9 | 50 | GTCTGGCGGCNDTACCNDTTACACAGACAGCGT |
| | | AAAAGGCCGTTTCACAATC |
| Pri10 | 51 | GGTAHNGCCGCCAGACSNAHNAATAGTCGCCAC |
| | | AAACTCACGTTCTTTCCCC |
| PriPep-F | 52 | CTAGCCCCGCGAAATTAATACGACT |
| PriPep-R | 53 | CCTCCTGCAGGTTAACCTTACTCGA |
| PylT tRNA(−CA)_{CUA} | 54 | GGAAACCUGAUCAUGUAGAUCGAACGGACUCU |
| | | AAAUCCGUUCAGCCGGGUUAGAUUCCCGGGGU |
| | | UUCCGC |
| Mycoplasma | 55 | GGGAGAGUAGUUCAAUGGUAGAACGUCGGUC |
| capricolum Trp1 | | UCUAAAACCGAGCGUUGAGGGUUCGAUUCCUU |
| RNA(−CA)_{CUA} | | UCUCUCCCAC |
| Pri11 | 56 | TTAATACGACTCACTATAGGGTCTAGAAATAAT |
| | | TTTGTTTAACTTTAAGAAGGAGATATACATATG |
| | | CAAGTGCAGTTACAAGAGTCGGGTGG |
| Pri12 | 57 | ACCAGAACCACCACCAGAACCATGATGATGATG |
| | | GTGATGGCCGCTTCCATGATGGTGGTGATGGTG |
| | | ACCACCAGATCCTGAGCTTACGG |
| Pri13 | 58 | ACCAGAACCACCACCAGAACCATGATGATG |
| Pri14 | 59 | GTTTAACTTTAAGAAGGAGATATACATATGCAA |
| | | GTGCAGTTACAAGAGTCGGGTG |
| Pri15 | 60 | CTTACTCGAGAATTCCCGGGATCCTCATTAACC |
| | | AGAACCACCACCAGAACCATGATGATG |
| Pri16 | 61 | TAATGAGGATCCCGGGAATTC |
| Pri17 | 62 | CATATGTATATCTCCTTCTTAAAGTTAAACAAA |
| | | ATTATTTC |

### REFERENCES

1. Leisle, L. et al. Cellular encoding of Cy dyes for single-molecule imaging. eLife 5, e19088 (2016).
2. Taira, H., Matsushita, Y., Kojima, K., Shiraga, K. & Hohsaka, T. Comprehensive screening of amber suppressor tRNAs suitable for incorporation of non-natural amino acids in a cell-free translation system. Biochemical and Biophysical Research Communications 374, 304-308 (2008).
3. Wrapp, D. et al. Structural Basis for Potent Neutralization of Betacoronaviruses by Single-Domain Camelid Antibodies. Cell 181, 1004-1015.e1015 (2020).
4. Kuru, E. et al. Release Factor Inhibiting Antimicrobial Peptides Improve Nonstandard Amino Acid Incorporation in Wild-type Bacterial Cells. ACS Chemical Biology 15, 1852-1861 (2020).
5. Yonker, L. M. et al. Multisystem inflammatory syndrome in children is driven by zonulin-dependent loss of gut mucosal barrier. The Journal of Clinical Investigation 131 (2021).
6. Shevchenko, A., Wilm, M., Vorm, O. & Mann, M. Mass spectrometric sequencing of proteins silver-stained polyacrylamide gels. Anal Chem 68, 850-858 (1996).
7. Peng, J. & Gygi, S. P. Proteomics: the move to mixtures. J Mass Spectrom 36, 1083-1091 (2001).
8. Eng, J. K., McCormack, A. L. & Yates, J. R. An approach to correlate tandem mass spectral data of peptides with amino acid sequences in a protein database. J Am Soc Mass Spectrom 5, 976-989 (1994).
9. Doshi, R. et al. In vitro nanobody discovery for integral membrane protein targets. Scientific Reports 4, 6760 (2014).
10. Bushnell, B., Rood, J. & Singer, E. BBMerge—Accurate paired shotgun read merging via overlap. PLOS ONE 12, e0185056 (2017).
11. Hirshberg, M. et al. Crystal Structure of Phosphate Binding Protein Labeled with a Coumarin Fluorophore, a Probe for Inorganic Phosphate. Biochemistry 37, 10381-10385 (1998).
12. Tsai, Y. C., Jin, Z. & Johnson, K. A. Site-specific labeling of T7 DNA polymerase with a conformationally sensitive fluorophore and its use in detecting single-nucleotide polymorphisms. Analytical biochemistry 384, 136-144 (2009).
13. Kim, Y. E., Chen, J., Chan, J. R. & Langen, R. Engineering a polarity-sensitive biosensor for time-lapse imaging of apoptotic processes and degeneration. Nature Methods 7, 67-73 (2010).
14. Brient-Litzler, E., Plückthun, A. & Bedouelle, H. Knowledge-based design of reagentless fluorescent biosensors from a designed ankyrin repeat protein. Protein engineering, design & selection: PEDS 23, 229-241 (2010).
15. de Lorimier, R. M. et al. Construction of a fluorescent biosensor family. Protein Sci 11, 2655-2675 (2002).

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method of selectively acylating pdCpA at the 2′-OH and/or 3′-OH positions comprising reacting pdCpA with an acylimidazole, wherein the step of reacting is carried out in a solvent comprising water.

2. A method of selectively acylating pdCpA at the 2′-OH and/or 3′-OH positions to form the following: 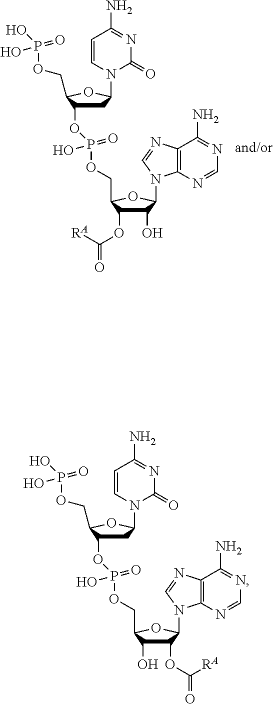
or salts thereof, the method comprising:
(a) a step of reacting a compound of the formula: R^{A}(═O)OH, or a salt thereof, with carbonyldiimidazole (CDI) to form a compound of Formula (A): 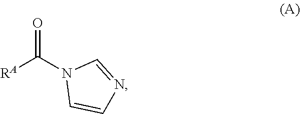
or a salt thereof; and
(b) a step of reacting the compound of Formula (A), or a salt thereof, with pdCpA: 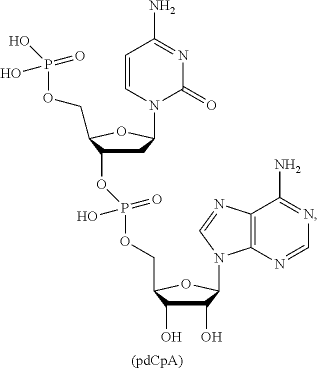
or a salt thereof,
wherein step (b) of reacting is carried out in a solvent comprising water; and
wherein R^{A} is an organic small molecule.

3. The method of claim 2, wherein R^{A} is of the formula: 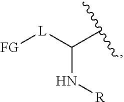
wherein:
FG is a fluorogenic small molecule;
L is a bond or a linker; and
R is hydrogen or a nitrogen protecting group.

4. A method of preparing a compound of Formula (I): 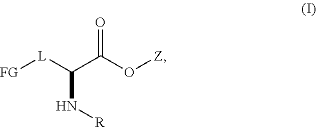
or a salt, stereoisomer, or tautomer thereof, wherein:
FG is a fluorogenic small molecule;
L is a bond or a linker;
R is hydrogen or a nitrogen protecting group; and
Z is a nucleotide;
comprising coupling a compound of Formula (II): 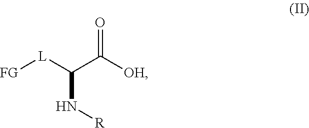
or a salt, stereoisomer, or tautomer thereof, with a nucleotide.

5. The method of claim 4, wherein the compound of Formula (II) is coupled selectively at the 2′-OH and/or 3′-OH position of the nucleotide.

6. The method of claim 4 or 5, wherein Z is a mononucleotide, dinucleotide or polynucleotide.

7. The method of any one of claims 4-6, wherein Z is a dinucleotide.

8. The method of any one of claims 4-7, wherein Z is pdCpA.

9. The method of any one of claims 4-8, wherein Z is of the formula:

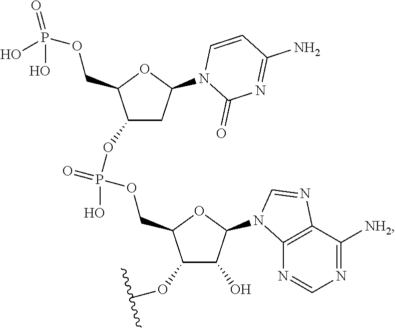 [SMILES: *O[C@H]1[C@@H](O)[C@H](n2cnc3c(N)ncnc32)O[C@@H]1COP(=O)(O)O[C@H]1C[C@H](n2ccc(N)nc2=O)O[C@@H]1COP(=O)(O)O]

10. The method of any one of claims 4-8, wherein Z is of the formula:

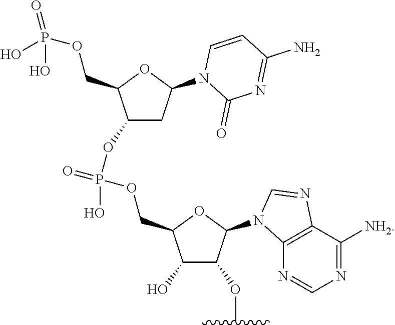 [SMILES: *O[C@@H]1[C@H](O)[C@@H](COP(=O)(O)O[C@H]2C[C@H](n3ccc(N)nc3=O)O[C@@H]2COP(=O)(O)O)O[C@H]1n1cnc2c(N)ncnc21]

11. The method of any one of claims 4-10, the method comprising:
(a) a step of reacting a compound of Formula (II): 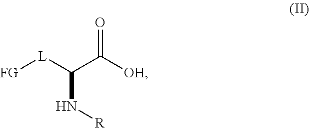
or a salt, stereoisomer, or tautomer thereof, with carbonyldiimidazole (CDI) to form a compound of Formula (A′): 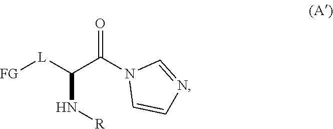
or a salt, stereoisomer, or tautomer thereof; and
(b) a step of reacting the compound of Formula (A′), or a salt, stereoisomer, or tautomer thereof, with the nucleotide.

12. The method of any one of claims 4-11, wherein the coupling is carried out in a solvent comprising water.

13. The method of claim 11, wherein the step (b) of reacting is carried out in a solvent comprising water.

14. The method of any one of the preceding claims, wherein the solvent comprising water comprises a mixture of water and a second solvent.

15. The method of claim 14, wherein the second solvent is DMF.

16. The method of any one of the preceding claims, wherein the solvent comprising water is a mixture of DMF and water.

17. The method of claim 16, wherein the ratio of DMF:water is from 30:70 to 60:40 by volume.

18. The method of claim 16, wherein the ratio of DMF:water is from 40:60 to 50:50 by volume.

19. The method of claim 16, wherein the ratio of DMF:water is about 45:55 by volume.

20. The method of any one of the preceding claims, wherein the solvent comprising water has a pH of greater than 7.

21. The method of any one of the preceding claims, wherein the solvent comprising water has a pH of about 7 to about 9.

22. The method of any one of the preceding claims, wherein the solvent comprising water has a pH of about 7.5 to about 8.5.

23. The method of any one of the preceding claims, wherein the solvent has a pH of about 8.

24. The method of any one of claims 4-23 further comprising deprotecting a compound of Formula (I): 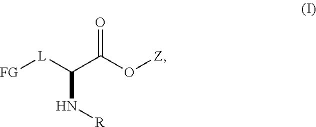
or a salt, stereoisomer, or tautomer thereof, wherein:
FG is a fluorogenic small molecule;
L is a bond or a linker;
R is a nitrogen protecting group; and
Z is a nucleotide;
to yield a compound of Formula (III): 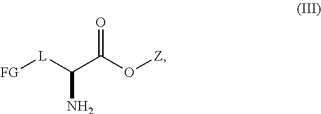
or a salt, stereoisomer, or tautomer thereof.

25. The method of claim 24, wherein the step of deprotecting is carried out in the presence of an acid.

26. The method of any one of claims 4-25, wherein R is a carbamate protecting group.

27. The method of any one of claims 4-26, wherein R is a tert-Butyloxycarbonyl (Boc) protecting group.

28. The method of any one of claims 25-27, wherein the acid is an organic acid.

29. The method of any one of claims 25-28, wherein the acid is a carboxylic acid.

30. The method of any one of claims 25-29, wherein the acid is trifluoro acetic acid.

31. The method of any one of the preceding claims, wherein FG is of one of the following formulae: 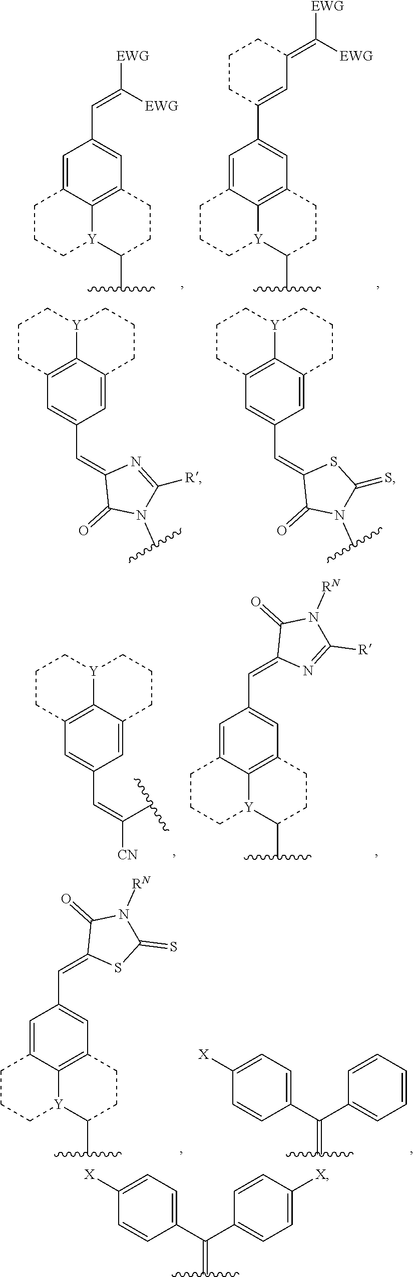 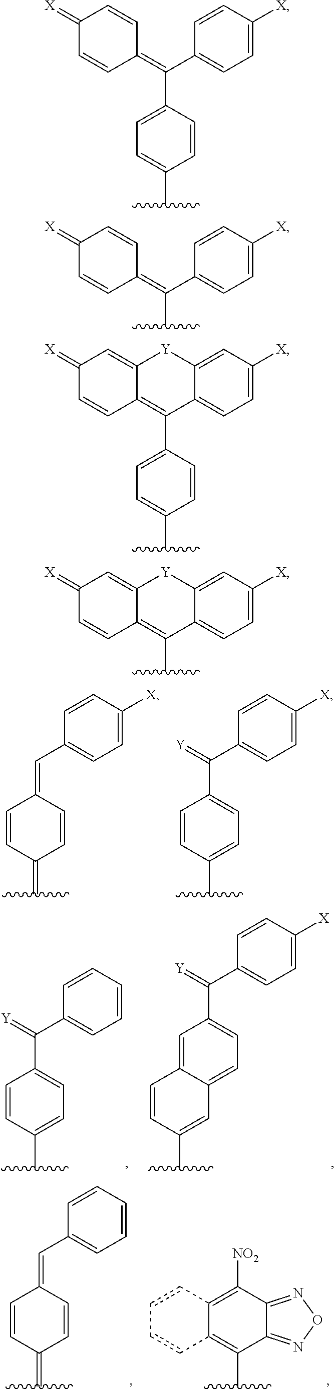 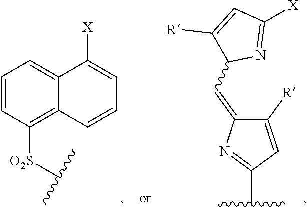
or a salt, stereoisomer, or tautomer thereof; wherein:
each instance of EWG is independently an electron withdrawing group;
Y is N, —NR^{N}, O, S, or —C(R′)₂;
each instance of X is independently —N(R^{N})₂, —OR^{O}, or —SR^{S};
each instance of R′ is independently hydrogen, halogen, —CN, —NO₂, —N₃, —N(R^{N})₂, —OR^{O}, —SR^{S}, alkyl, alkenyl, alkynyl, carbocyclyl, aryl, heterocyclyl, heteroaryl, acyl, sulfinyl, or sulfonyl; and
each instance of R^{N}, R^{O}, and R^{S} is independently hydrogen, alkyl, alkenyl, alkynyl, carbocyclyl, aryl, heterocyclyl, heteroaryl, or acyl; and
wherein each formula is further optionally substituted.

32. The method of any one of the preceding claims, wherein FG comprises one of the following:

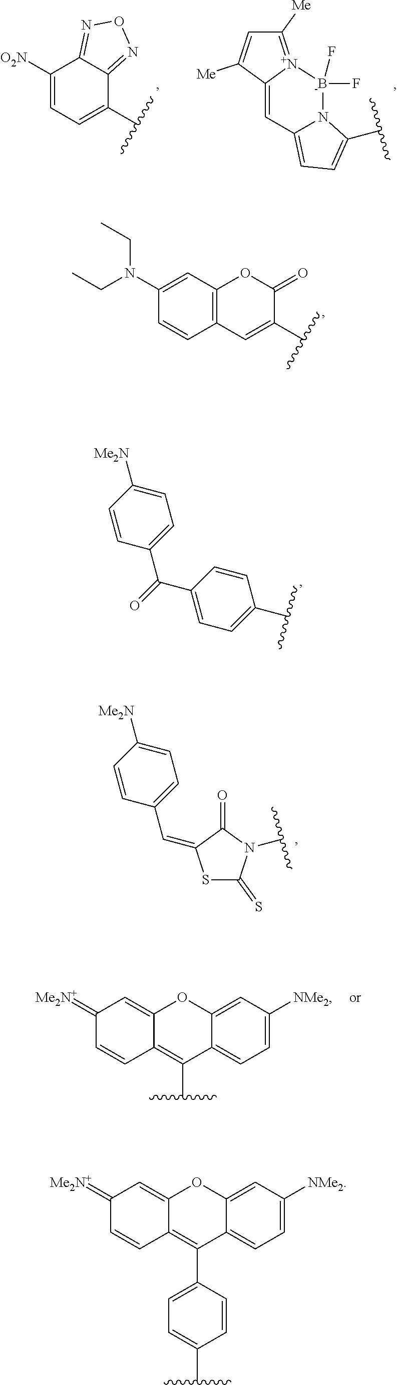

33. The method of any one of the preceding claims, wherein -L- is of one of the following formulae: 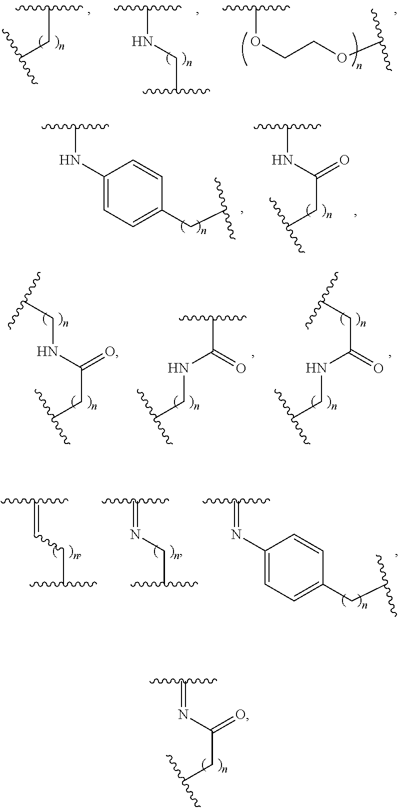
or a salt, stereoisomer, or tautomer thereof; wherein:
each n is independently 0 or an integer from 1-20, inclusive; and
wherein each formula is further optionally substituted.

34. The method of any one of the preceding claims, wherein the compound of Formula (II) is one of the following: 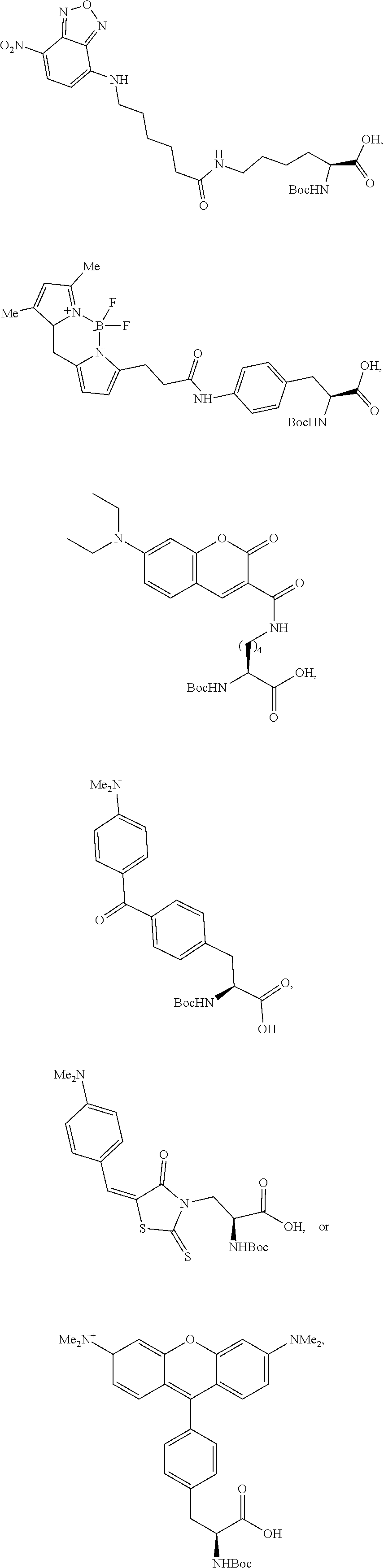
or a salt, stereoisomer, or tautomer thereof.

35. The method of any one of the preceding claims, wherein the compound of Formula (I) is one of the following: 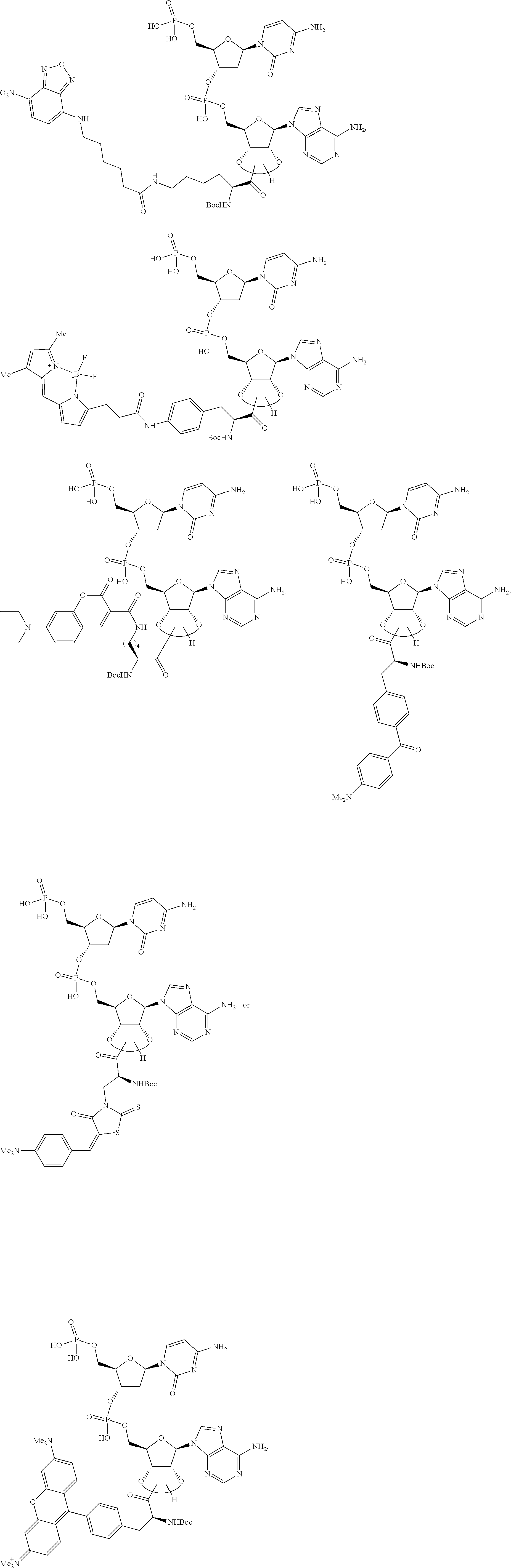
or a salt, stereoisomer, or tautomer thereof.

36. The method of any one of the preceding claims, wherein the compound of Formula (III) is one of the following: 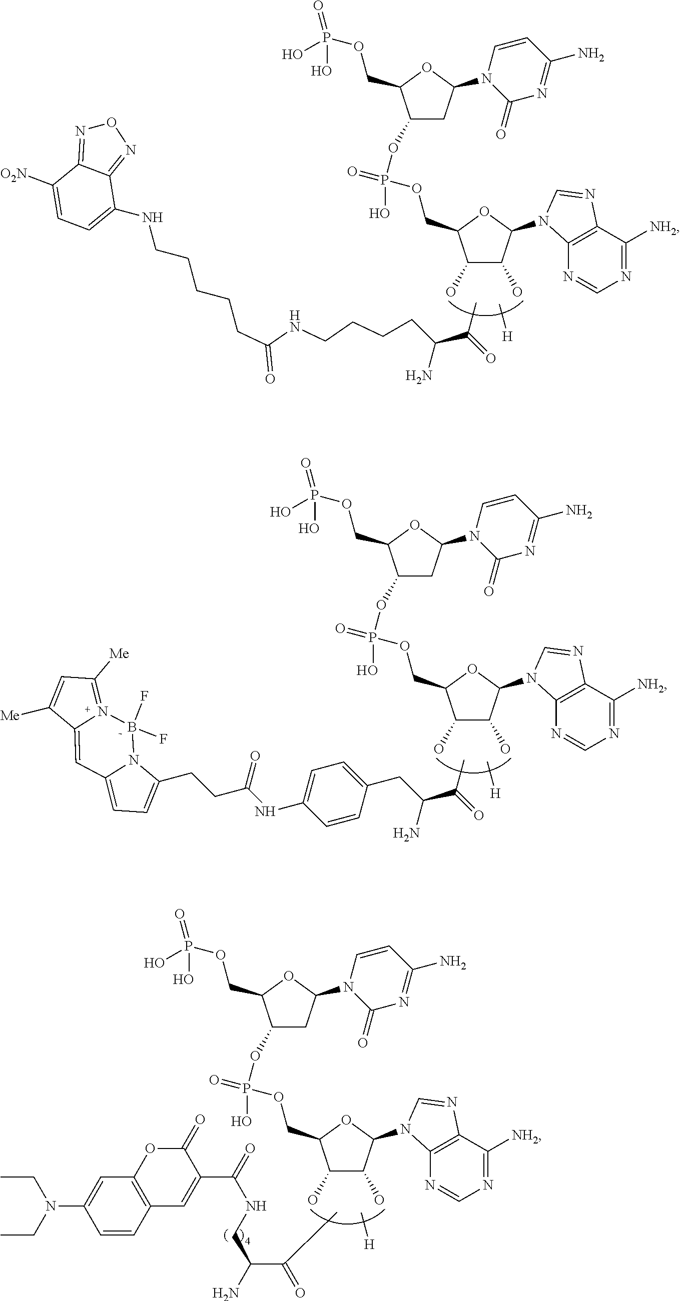 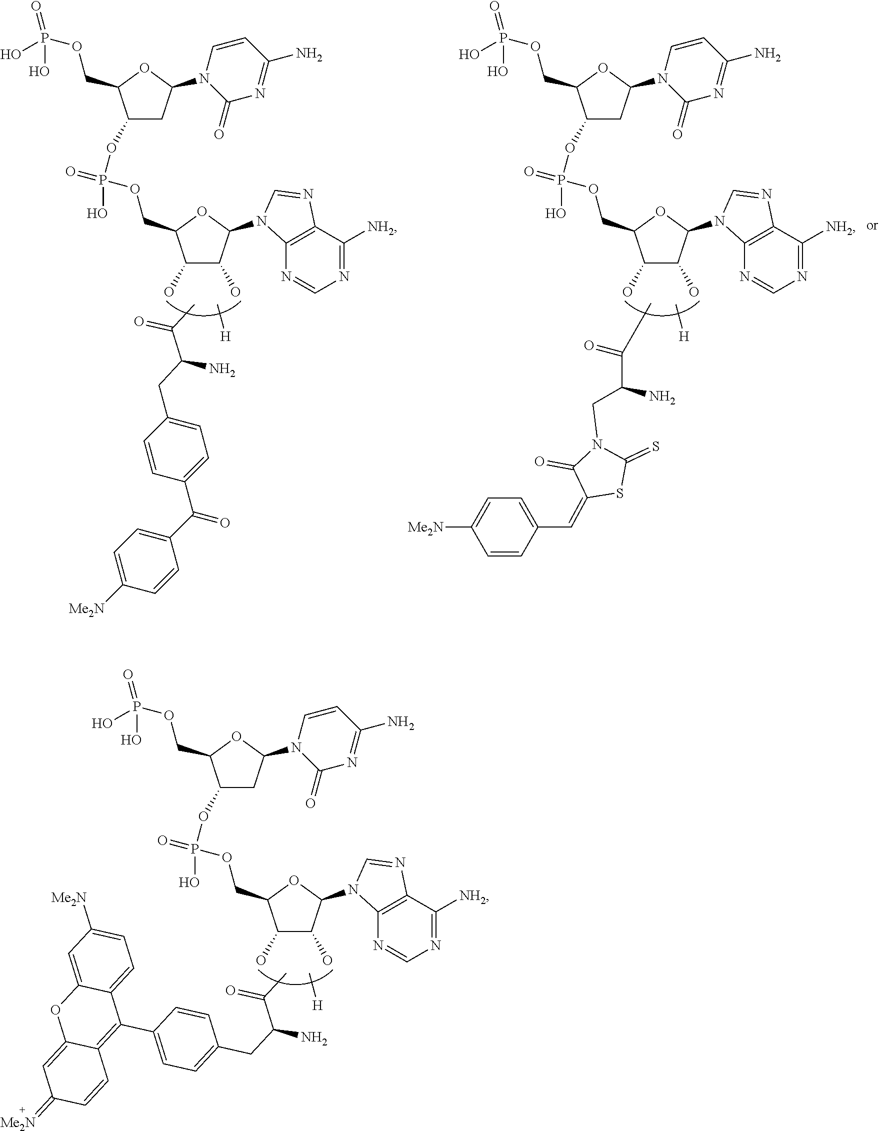
or a salt, stereoisomer, or tautomer thereof.

37. A fluorogenic sensor for detecting a target comprising a nanobody, wherein the nanobody comprises an amino acid sequence with at least 90% sequence identity with any one of SEQ ID NOs: 1-3 and 7-10:
(SEQ ID NO: 1)
QVQLQESGGGLVQAGGSLRLSCAASGRTFSEYAMGWFRQAPGREREFVA
TIGPSGGVTGYTDSVRGRFTISRDNARNTVYLQMNSLRPDDTAVYYCAA
AGLGTXVSEWDYDYDYWGQGTQVTVSSGSGGGSGGSGGGSG;
(SEQ ID NO: 2)
QVQLQESGGGLVQAGGSLRLSCAASGRTFSEYAMGWFRQAPGREREFVA
TIGPSGGITGYTDSVRGRFTISRDNARNTVYLQMNSLRPDDTAVYYCAA
AGLGTXVSEWDYDYDYWGQGTQVTVSSGSGGGSGGSGGGSG;
(SEQ ID NO: 3)
QVQLQESGGGLVQAGGSLRLSCAASGRTFSEYAMGWFRQAPGREREFVA
TILRSGGSTFYTDSVRGRFTISRDNARNTVYLQMNSLRPDDTAVYYCAA
AGLGTXVSEWDYDYDYWGRGTQVTVSSGSGGGSGGSGGGSG;
(SEQ ID NO: 7)
MQVQLQESGGGLVQAGGSLRLSCAASGRTFSEXAMGWFRQAPGREREFV
ATISWSGGSTYYTDSVRGRFTISRDNARNTVYLQMNSLRPDDTAVYYCA
AAGLGTVVSEWDYDYDYWGQGTQVTVSSGSGGGSGGSGGGSG;
(SEQ ID NO: 8)
MQVQLQESGGGLVQAGGSLRLSCAASGRTFSEYAMGWFRQAPGREREFV
ATISWSGGXTYYTDSVRGRFTISRDNARNTVYLQMNSLRPDDTAVYYCA
AAGLGTVVSEWDYDYDYWGQGTQVTVSSGSGGGSGGSGGGSG;
(SEQ ID NO: 9)
MKIEEQVQLQESGGGLVQAGGSLRLSCAASGRTFSEYAMGWFRQAPGKE
REFVATIGPSGGCTGYTDSVKGRFTISRDNAKNTVYLQMNSLKPDDTAV
YYCAAAGLGTXVSEWDYDYDYWGQGTQVTVSSGSGGGSGGSGGGSG;
(SEQ ID NO: 10)
MKIEEQVQLQESGGGLVQAGGSLRLSCAASGRTFSEYAMGWFRQAPGKE
REFVATIGPSGGITYYTDSVKGRFTISRDNAKNTVYLQMNSLKPDDTAV
YYCAAAGLGVXISEWDYDYDYWGQGTQVTVSSGSGGGSGGSGGGSG;
wherein X is a fluorogenic amino acid or amino acid conjugated to a fluorogenic small molecule.

38. The fluorogenic sensor of claim 37, wherein the nanobody comprises an amino acid sequence at least 91%, 92%, 93%, 94%, 95%, 96%, 97%, 98%, or 99% identical to any one of SEQ ID NOs: 1-3 and 7-10.

39. The fluorogenic sensor of claim 37, wherein the nanobody comprises an amino acid sequence with 100% sequence identity with any one of SEQ ID NOs: 1-3 and 7-10.

40. A fluorogenic sensor for detecting a target comprising a nanobody, wherein the nanobody comprises an amino acid sequence with at least 90% sequence identity with SEQ ID NO: 4:
(SEQ ID NO: 4)
QVQLVESGGGLMQAGGSLRLSCAVSGXTFSTAAMGWFRQAPGREREFVA
AIRWSGGSAYYADSVRGRFTISRDRARNTVYLQMNSLRYEDTAVYYCAQ
THYVSYLLSDYATWPYDYWGQGTQVTVSSGSGGSGGGSGGGSG;
wherein X is a fluorogenic amino acid or an amino acid conjugated to a fluorogenic small molecule.

41. The fluorogenic sensor of claim 40, wherein the nanobody comprises an amino acid sequence at least 91%, 92%, 93%, 94%, 95%, 96%, 97%, 98%, or 99% identical to SEQ ID NO: 4.

42. The fluorogenic sensor of claim 40, wherein the nanobody comprises an amino acid sequence with 100% sequence identity with SEQ ID NO: 4.

43. The fluorogenic sensor of any one of claims 37-42, wherein the nanobody binds a spike protein of a coronavirus or variant thereof.

44. The fluorogenic sensor of any one of claims 37-43, wherein the nanobody binds a spike protein of a SARS-CoV-2 virus or variant thereof.

45. The fluorogenic sensor of any one of claims 37-44, wherein the nanobody binds a spike protein of an Omicron variant of a SARS-CoV-2 virus.

46. A fluorogenic sensor for detecting an EGFR protein comprising:
a nanobody that binds an epidermal growth factor receptor (EGFR) protein; and
a fluorogenic small molecule conjugated to a target-binding domain of the nanobody.

47. The fluorogenic sensor of claim 46, wherein the nanobody comprises an amino acid sequence with at least 80% sequence identity with SEQ ID NO: 5:
(SEQ ID NO: 5)
QVQLQESGGGLVQPGGSLRLSCAASGRTFSSYAMGWFRQAPGKQREFVA
AIRWSGGYTYYTDSVKGRFTISRDNAKTTVYLQMNSLKPEDTAVYYCAA
TYLSSDYSRYALPQRPLDYDYWGQGTQVTVSSGSGGSGGGSGGGSG

48. The fluorogenic sensor of 46 or 47, wherein the nanobody comprises an amino acid sequence with at least 80% sequence identity with SEQ ID NO: 6:
(SEQ ID NO: 6)
QVQLQESGGGLVQPGGSLRLSCAASGRTFSXYAMGWFRQAPGKQREFVA
AIRWSGGYTYYTDSVKGRFTISRDNAKTTVYLQMNSLKPEDTAVYYCAA
TYLSSDYSRYALPQRPLDYDYWGQGTQVTVSSGSGGSGGGSGGGSG;
wherein X is a fluorogenic amino acid or an amino acid conjugated to a fluorogenic small molecule.

49. The fluorogenic sensor of any one of claims 46-48, wherein the nanobody comprises an amino acid sequence at least 85%, 90%, 91%, 92%, 93%, 94%, 95%, 96%, 97%, 98%, or 99% identical to SEQ ID NO: 5 or 6.

50. The fluorogenic sensor of any one of claims 46-48, wherein the nanobody comprises an amino acid sequence with 100% sequence identity with SEQ ID NO: 5 or 6.

51. A method of detecting a target, the method comprising:
contacting a target with a fluorogenic sensor of any one of the preceding claims; and
measuring or observing the fluorescence of the fluorogenic sensor, or measuring or observing a change in the fluorescence lifetime of the fluorogenic sensor.

52. The method of claim 51, wherein a change in fluorescence and/or fluorescence lifetime is observed instantaneously after the contacting step.

53. The method of claim 51, wherein the change in fluorescence and/or fluorescence lifetime is observed within less than 1 second after the contacting step.

54. The method of claim 51, wherein change in fluorescence and/or fluorescence lifetime is observed within less than less than 2500, 2000, 1500, 1000, 750, 500, or 250 milliseconds (ms) after the contacting step.

55. The method of any one of claims 51-54, wherein an increase in fluorescence of at least 1-fold, 2-fold, 5-fold, 10-fold, 20-fold, 25-fold, 30-fold, 35-fold, 40-fold, 45-fold, 50-fold, 100-fold, 150-fold, 200-fold, 300-fold, 400-fold, or 500-fold is observed.

56. A kit comprising a fluorogenic sensor of any one of the preceding claims, and optionally instructions for use.

57. A kit for preparing tRNA charged with one or more a non-standard amino acids (nsAAs), wherein the kit comprises: (i) pdCpA; and (ii) one or more nsAAs.

58. The kit of claim 57, wherein the kit further comprises (iii) CDI.

59. The kit of claim 57 or 58, wherein the kit further comprises (iv) one or more buffered aqueous solutions.

60. The kit of any one of claims 57-59, wherein the kit comprises (v) a solid phase extraction filter.

61. The kit of any one of claims 57-60, wherein the one or more nsAAs are fluorogenic amino acids (FgAAs).

62. A method of preparing a fluorogenic sensor by protein translation comprising a step of mRNA protein translation in the presence of a tRNA, wherein the tRNA is charged with one or more fluorogenic amino acids.

63. A method of selecting a fluorogenic sensor of interest, the method comprising:
(i) obtaining or preparing one or more candidate fluorogenic sensors;
(ii) contacting the candidate fluorogenic sensors with a target;
(iii) selecting a candidate fluorogenic sensor as a fluorogenic sensor of interest if the change in fluorescene or change in the fluorescence lifetime of the fluorogenic sensor is above a certain threshold.
